(19) United States
(12) Patent Application Publication
(10) Pub. No.: US 2017/0368167 A9
(43) Pub. Date: Dec. 28, 2017

(54) COMPOSITIONS AND METHODS RELATED TO NEUROLOGICAL DISORDERS

(71) Applicants: The Institute for Molecular Medicine, Huntington Beach, CA (US); VAXINE PTY LTD, Adelaide (AU)

(72) Inventors: Michael Agadjanyan, Huntington Beach, CA (US); Anahit Ghochikyan, Huntington Beach, CA (US); Nikolai Petrovsky, Adelaide (AU)

(21) Appl. No.: 15/174,709

(22) Filed: Jun. 6, 2016

Related U.S. Application Data
(63) Continuation-in-part of application No. 14/628,023, filed on Feb. 20, 2015.
Application No. 14/628,023 is a continuation of application No. PCT/US2013/055877, filed on Aug. 20, 2013.
(60) Provisional application No. 61/691,607, filed on Aug. 21, 2012.
(60) Provisional application No. 61/792,770, filed on Mar. 15, 2013.

(51) Int. Cl. A61K 39/39; A61K 39/00

(52) U.S. Cl. CPC A61K 39/39; A61K 39/0007; A61K 2039/55583; A61K 2039/55505; A61K 2039/575; A61K 2039/55561

(57) ABSTRACT

The present technology relates to compositions comprising inulin particles for use in the enhancement of immune responses to neuronal self-antigens for treating or preventing neurodegenerative diseases, in a subject. Also provided are pharmaceutically acceptable compositions comprising: particles of inulin; a substance comprising one or more pathogen-associated molecular patterns (PAMPs); and a neuronal self-antigen fused to carrier, and methods and uses of the composition for inducing or modulating an immune response in a subject, such as modulating an immune response to a neuronal self-antigen as a vaccine. Also provided are vaccine compositions comprising inulin particles, and an antigen-binding carrier material, and methods and uses of the vaccine.

### RELATED APPLICATIONS

This application is a Continuation-In-Part of pending U.S. application Ser. No. 14/628,023, filed Feb. 20, 2015, as the national stage of International Application PCT/US2013/055887, filed in the United States on Aug. 20, 2013, which in turn claims benefit under 35 USC section 119(e) of provisional application 61/792,770, filed Mar. 15, 2013 and of provisional application 61/691,607. This application is also a Continuation-In-Part of pending U.S. application Ser. No. 14/127,489, filed Dec. 19, 2013, as the national stage of International Application PCT/EP2012/061748, filed in the EP on Jun. 19, 2012.

### BACKGROUND

Traditional vaccination against infectious diseases relies on generation of cellular and humoral immune responses that act to protect the host from overt disease even though they do not induce sterilizing immunity. More recently, attempts have been made with mixed success to generate therapeutic vaccines against a wide range of noninfectious diseases including neurodegenerative disorders such as Alzheimer's disease (AD), Parkinson disease (PD), Dementia with Lewy Bodies Dementia (DLB), Frontotemporal Dementia (FTD), Traumatic Brain Injury (TBI), etc. Strategies on development of vaccines against neurodegenerative diseases are based on the generation of humoral immune responses against mutated or altered self-proteins that are hallmarks of the diseases. However, immunological tolerance to self-antigens, though altered, make difficult the generation of potent immune responses allowing the production of therapeutically relevant concentrations of antibodies specific to pathological self-molecules, such as amyloid-β (Aβ), tau, α-synuclein, etc. To achieve this goal and generate high concentrations of therapeutically potent antibodies one should find a safe composition of an immunogenic non-self vaccine platform for delivery of self-antigen with a strong adjuvant.

The most prevalent form of dementia worldwide is Alzheimer's disease (AD). Compared to other deadly diseases, Alzheimer's is the only disease that cannot yet be prevented, cured or slowed. While death rates for other major diseases, such as heart diseases, cancer, AIDS etc, have declined, death rates from Alzheimer's disease have risen 66 percent since 2000 (www.alz.org).

AD is clinically characterized by progressive loss of memory, behavior impairment and decline of cognitive function. According to the World Health Organization (WHO), approximately 18 million people worldwide have Alzheimer's disease. By 2025, this estimate is projected to grow to 34 million people, with the highest increase expected among developing countries.

Neuropathological features of AD, and other neurodegenerative diseases, include neurofibrillary tangles, deposition of misfolded proteins in plaques and neuronal loss in affected brain regions. These pathological changes result in a profound loss of neurons and synapses over the course of the disease, thereby contributing to a progressive reduction in the functional capacity of the patient.

Since the “amyloid cascade hypothesis” was proposed, most therapeutic approaches for AD have focused on reducing amyloid-β (Aβ) levels in the brain, e.g., by blocking the formation of Aβ, promoting its clearance, preventing aggregation and destabilizing its oligomers. Anti-Aβ immunotherapy is considered as one of the most promising approaches in AD treatment and is currently being tested in clinical trials. Unfortunately, none of these attempts reported to date have shown positive clinical outcome. The first clinical trial of an AD vaccine, AN-1792, which used fibrillar Aβ₄₂ formulated in Th1 saponin-based adjuvant (QS21) was halted when 6% of the trial subjects receiving the active vaccine developed some degree of aseptic meningoencephalitis. It is hypothesized that the vaccine adjuvant used in this clinical vaccine trial as well as the associated activation of Aβ T cell epitopes (i.e. resulting in potential Th1 autoimmune responses), may have been major mediators of this severe side effect. At the same time, lessons learned from clinical trials indicate that to be effective, anti-Aβ immunotherapy should be initiated before cognitive decline and severe pathological changes have occurred and that clearing Aβ at the late stages may be insufficient to halt the progression of AD. In as much as pathological tau correlates much better with the degree of dementia than Aβ deposition, targeting tau is now considered a promising approach for the treatment of advanced AD stages. In addition, tau is a common pathological marker for several neurodegenerative disorders other than AD, categorized as tauopathies and therapeutics aimed at eliminating pathological tau may also be beneficial these diseases that include Amyotrophic Lateral Sclerosis, Frontotemporal Dementia with Parkinsonism linked to chromosome 17, Pick's Disease, Progressive Supranuclear Palsy, Creutzfeldt-Jakob Disease, Dementia Pugilistica, Down's Syndrome and others. Currently two vaccines targeting Tau entered phase 1 clinical trials, ACI-35 and AADVac1. ACI-35 is a liposome-based vaccine containing MPLA as an adjuvant and activator of innate immune system, whereas AADVac1 contain aluminum hydroxide (Alhydrogel) as an adjuvant.

Another antigen associated with neurodegenerative diseases is alpha-synuclein (α-Syn) that was first implied to neurodegeneration after identification of its presence in amyloid plaques of AD (Ueda K, 1993, PNAS). α-Syn was found in Lewy bodies (LBs), distrophic neurites and surrounding the core of the amyloid plaques. Synucleinopathies comprise a class of neurodegenerative diseases that share a morphologic hallmark, which is pivotally characterized by the involvement of Lewy pathology in a subset of neurons and glia. The synucleinopathies include PD, DLB, Multiple System Atrophy (MSA) and Pure Autonomic Failure (PAF). PD and DLB are the most prevalent neurodegenerative disorders, after AD, and it is with these conditions that intracytoplasmic LBs and dystrophic LNs are most commonly associated. Notably, up to 50% of AD cases exhibit Lewy bodies, and the presence of Lewy body pathology in AD is associated with a more aggressive disease course and accelerated cognitive dysfunction. Mixed brain pathologies account for most dementia cases in community-dwelling older persons and there are multiple reports on the interactions of amyloidogenic proteins. Overlap of clinical and neuropathological features of AD and PD are observed in dementia with DLB, and molecular interactions between α-syn and Aβ were directly demonstrated by NMR. In addition, it was shown that Aβ interacted directly with α-syn and stabilized the formation of hybrid nanopores that alter neuronal activity and might contribute to AD. Thus, careful neuropathological studies have shown that aggregations of α-syn, Aβ and also tau appear in the same neuronal structures, providing a pathological basis for the clinical observations of the overlap between PD/DLB and AD.

Several pre-clinical studies have demonstrated α-Syn oligomer/aggregate clearance using immunotherapy, including active immunization. The first phase I clinical trial for immunotherapy against α-Syn began in 2012. Developed by AFFiRiS, the AFFiTOPE, PD01, improved α-Syn-induced pathology, including neuronal loss in mice, although data on human trials are not published yet.

The common link between all these neurodegenerative diseases is chronic activation of innate immune responses including those mediated by microglia, the resident CNS macrophages. Along with controlling inflammatory processes, and repair and regeneration, activation of microglia can trigger neurotoxic pathways leading to progressive degeneration. The adaptive immune response in neurodegenerative diseases may serve as double edge sword contributing to tissue damage or resolving inflammation and mediating neuroprotection and repair. In case of vaccination-induced pro-inflammatory immune responses additional inflammation may be crucial for neurons that have only a limited capacity for repair and cannot tolerate long-term inflammation.

This means that, successful adjuvant-antigen vaccine combinations should be found that will be effective in generation of humoral responses inducing anti-inflammatory responses and avoiding induction of additional pro-inflammatory responses. Even after extensive validation in animal models, adjuvant-antigen combinations that were effective in animal challenge models may be ineffective in generation of antibody responses to self-antigens or even detrimental when administered to humans. An example of the former is the ineffectiveness of alum adjuvants in human AD vaccines CAD106, AD03, LU AF20513 that are in various stages of clinical trials, despite showing enhanced protection in animal models. Another example is AN-1792 trial using QS21 adjuvant, showing no adverse effects in animal models but supposedly increased the adverse events in patients after adding emulsifier.

β-D-(2-1) polyfructofuranosyl α-D-glucose (commonly known as inulin) is a polysaccharide that (as disclosed by WO 87/02679, WO 2006/024100, and WO 2011/032229, the contents of each of which are incorporated herein by reference) develops useful properties when crystallized into stable particulate structures. Inulin has a relatively hydrophobic, polyoxyethylene-like backbone, and this unusual structure plus its non-ionized nature allows re-crystallization and easy preparation in a very pure state. Inulin in its raw state is generally soluble in warm water but, as disclosed by WO 87/02679, WO 2006/024100, WO 2011/032229 and WO 2012175518 can with specific treatments be crystallized into more stable polymorphic forms, including the previously described gamma (gIN), delta (dIN) and epsilon (eIN) forms.

Such inulin particles (hereinafter collectively referred to simply as ‘inulin particles’) are largely insoluble at normal mammalian body temperature and have been found to possess excellent adjuvant properties when formulated with antigens.

As described further in the present application, when studying the biological effects of inulin particles, the surprising discovery has now been made that inulin particles formulated with vaccines targeting self-molecules involved in Alzheimer's Disease and Parkinson Disease pathologies induced unexpectedly and incredibly strong T and B cell mediated immune responses compared with other adjuvants approved by FDA or used in clinical trials so far. To test whether inulin particles could enhance immune responses to vaccines against different neurodegenerative diseases, they were mixed with a universal vaccine platform for delivering self-antigens, such as Aβ, tau and synuclein. The combination of an inulin particle together with a PAMP innate immune activator and the vaccine against neurodegenerative diseases has been found to result in a surprisingly favorable and synergistic immune response without generation of detrimental pro-inflammatory reactions.

Microbial-derived compounds that trigger innate immune activation also enhance the adaptive immune response to a co-administered vaccine antigen. Such compounds are now known to comprise or mimic pathogen-associated molecular patterns (PAMPs), where a PAMP is a structurally conserved motif derived from a pathogen that is immunologically distinguishable from host molecules, and is recognized by an innate immune receptor. PAMPs are present in certain types of protein, lipid, lipoprotein, carbohydrate, glycolipid, glycoprotein, and nucleic acids expressed by particular pathogens and include triacyl lipopeptides, porins, glycans, single and double stranded RNA, flagellin, lipotechoic acid, N-formymethionine, and bacterial or viral DNA, amongst others. PAMPs act as innate immune activators by binding to PAMP-specific innate immune receptors such as toll-like receptors (TLR), NOD-like receptors, RIG ligase receptors and C-type lectins. This leads to activation of inflammatory gene pathways in immune cells.

What these PAMP compounds have in common is that they all activate the innate immune system and induce inflammatory gene pathways, in particular through activating Nuclear Factor-Kappa B (NF□B), the master transcriptional regulator of inflammatory gene activation. This inflammation in turn may lead to enhancement of an adaptive immune response to a co-administered antigen, as a by-product or downstream effect of the innate immune activation. The enhancement by a separate substance of an adaptive immune response to a co-administered antigen is known as an “adjuvant” effect.

Without wishing to be restricted by theory, it is accepted by those skilled in the art that the common factor that links all compounds that possess adjuvant activity is that they induce immune “danger signals” leading to activation of innate immune and thereby activation of NF□B and other inflammatory pathways. Danger signals that provide immune adjuvant effects can be generated by local tissue damage, e.g., induced by injection of inflammatory substances such as oil emulsions, or more specifically through binding of PAMPs to innate immune receptors whose role is to detect pathogen invasion and tissue damage. PAMP-associated danger signals thereby alert the innate immune system of the need to mount a defensive inflammatory response against the perceived threat. Following the activation of these danger-sensing PAMP receptors, inflammatory gene signaling pathways including the key NFκB pathway are activated leading to secretion by immune cells such as monocytes of key inflammatory effectors including tumor necrosis factor (TNF)-α, interleukin (IL)-1, IL-6, IL-8 and IL-12, amongst others. These inflammatory mediators released in response to PAMP activation are believed in the art to be critical to the ability of PAMPs to enhance antigen-specific adaptive immune responses, with high-throughput cell-based screening assays designed to identify new adjuvant compounds reliant upon their ability to induce inflammatory cytokines such as TNF-α, IFN-γ, IL-1, IL-8 or IL12 as the readout of potential adjuvant activity.

Conceptually, two or more such innate immune activators when combined together induce even stronger danger signals, generate higher levels of inflammatory gene activation, and thereby are predicted to show increased adjuvant potency. However, as known by those skilled in the art, the problem of using PAMPs either singly or, more particularly, combined together as immune modulators or vaccine adjuvants is that the inflammatory effects are highly toxic and hence the ability to achieve enhancement of an adaptive immune response in this way is hindered by severe dose-limiting local and systemic inflammation-associated toxicity which is correspondingly magnified as the dose of the innate immune activator is increased. For example, even the combination of a partially detoxified PAMP analogue, monophosphoryl lipid A (MPL), with aluminum hydroxide (“alum”) adjuvant in a hepatitis B surface antigen (HBsAg) vaccine caused significantly more local injection site reactions, fever and other systemic side effects than HBsAg with alum adjuvant alone.

Increased vaccine reactogenicity and toxicity when two or more innate immune activators are combined in a vaccine formulation is a major barrier to regulatory approval of such adjuvant combinations, even where there might be a favorable impact on vaccine immunogenicity. Furthermore, not all combinations of innate immune activators are favorable from an immunogenicity standpoint, such that some combinations of innate immune activators produce an adaptive immune response to a co-administered antigen that is no better than the individual innate immune activator components alone, and some innate immune activator combinations even result in lower antigen-specific responses than with each individual innate immune activator used alone. For example, humans immunized with C-terminal recombinant malaria circumsporozoite antigen with alum alone achieved higher antigen-specific antibodies than subjects receiving the combination of alum with MPL.

The vaccine art recognizes the use of certain substances called adjuvants to potentiate an immune response when used in conjunction with an antigen. As used herein, the term “adjuvant” will be understood to mean any substance or material that when administered together or in conjunction with an antigen increases the immune response to that antigen. The problem with pure recombinant or synthetic antigens used in modern day vaccines is that they have poor immunogenicity when compared to less pure older-style live or killed whole cell vaccines. This has created a major need for development of effective adjuvants. Adjuvants are further used to elicit an immune response that is faster or greater than would be elicited without the use of the adjuvant. In addition, adjuvants may be used to create an immunological response using less antigen than would be needed without the inclusion of adjuvant, to increase production of certain antibody subclasses that afford immunological protection or to enhance particular cellular immune responses (e.g., CD4 or CD8 T cell memory responses).

Known adjuvants include aluminum salts (generically referred to as “alum” adjuvants). With few exceptions, alum adjuvants remains the only adjuvants licensed for human use in many countries. Although alum adjuvants are often useful to induce a good antibody (Th2) response to co-administered antigen(s), they are largely ineffective at stimulating a cellular (Th1) immune response, which are important for protection against many pathogens. Furthermore, alum has the potential to cause rare severe local and systemic side effects including sterile abscesses, eosinophilia and macrophagic myofasciitis. There is also community concern regarding the possible role of aluminum salts in neurodegenerative diseases such as Alzheimer's disease. Other licensed adjuvants including MF59, a squalene oil emulsion adjuvant that is licensed in Europe as part of an influenza vaccine and AS04, a combination of aluminum hydroxide and monophosphoryl lipid A (MPL), which is licensed in Europe in a hepatitis B vaccine.

However, the biggest single barrier to the development of improved human adjuvants whether used alone or together is the problem of local and systemic toxicity and adverse reactions. This is a particular problem for development of childhood vaccines where safety is paramount. Vaccine-mediated adverse reactions include inflammation and granuloma formation at the site of injection, pyrogenicity, nausea, adjuvant arthritis, uveitis, eosinophilia, allergy, anaphylaxis, organ specific toxicity or immunotoxicity, i.e. the liberation of toxic quantities of inflammatory cytokines. Such extreme toxicity hampers the use of otherwise highly potent adjuvants such as complete Freund's adjuvant (CFA), with this toxicity principally reflecting excessive activation of inflammatory pathways by innate immune activator adjuvants. Compounds or combined formulations that can successfully enhance adaptive immune responses, yet at the same time are well tolerated, safe and non-toxic to the host remain highly elusive, and of the hundreds of compounds known to be innate immune activators and possess vaccine adjuvant potential, less than a handful are approved for use in humans, and just two compounds, alum and MPL, being approved by the FDA for human vaccine use in the USA market.

Ideally, adjuvant formulations should be suited for use with a wide range of potential vaccine antigens and be safe for use in low responder populations including children, the elderly and immuno-compromised individuals. Thus, one of the major remaining challenges in vaccine research remains how to increase vaccine potency without inducing increased local or systemic toxicity. The difficulty of achieving this objective is exemplified by the fact alum adjuvants, 90 years after their discovery, continue to dominate human vaccine use.

Because for the most part the mechanisms of adjuvant action are not known, the art has generally not been able to predict on an empirical basis whether a particular compound, or mix of compounds, will have adjuvant activity. Similarly there is no way provided in the art to predict on an empirical basis whether a particular adjuvant, or mix of adjuvants, will be safe and well tolerated.

Moreover, each adjuvant-antigen composition may generate a different type of immune response, which may or may not provide enhanced protection against a relevant pathogen. For example, different types of adaptive immune response have been described, for example T helper(Th)1, Th2 and Th17 responses. For a particular pathogen, one adaptive immune response may be more favorable for providing protection than others. For example, for Leishmania a Th1 vaccine response is protective whereas a Th2 response may cause an unfavorable outcome. For other pathogens the converse may be true, such that a Th2 vaccine response is beneficial whereas a Th1 response is detrimental, and in even other situations a Th17 vaccine response may be desired.

This means that, in order to find successful adjuvant-antigen vaccine combinations, the art has relied on extensive trial and error testing. Even after extensive validation in animal models, examples abound of adjuvant-antigen combinations that were effective in animal challenge models and were ineffective or even detrimental when administered to humans. An example of the former is the ineffectiveness of alum adjuvants in human influenza vaccines, despite showing enhanced protection in animal models. Another example is respiratory syncytial virus (RSV) vaccine, which when formulated with alum adjuvant, enhanced immunogenicity and protection in animal models of RSV infection but caused worsened disease and increased deaths from RSV infection when administered to human children, an effect thought to be mediated by the vaccine inducing the wrong type of immune response, namely a Th2 rather than Th1 response.

β-D-(2-1) polyfructofuranosyl α-D-glucose (commonly known as inulin) is a polysaccharide that (as disclosed by WO 87/02679, WO 2006/024100, and WO 2011/032229) develops useful properties when crystallized into stable particulate structures. Inulin has a relatively hydrophobic, polyoxyethylene-like backbone, and this unusual structure plus its non-ionized nature allows re-crystallization and easy preparation in a very pure state. Inulin in its raw state is generally soluble in warm water but, as disclosed by WO 87/02679, WO 2006/024100 and WO 2011/032229, can with specific treatments be crystallized into more stable polymorphic forms, including the previously described gamma (gIN), delta (dIN) and epsilon (eIN) forms.

Such inulin particles (hereinafter collectively referred to simply as ‘inulin particles’) are largely insoluble at normal mammalian body temperature and have been found to possess excellent adjuvant properties. Without wishing to be bound by theory, the stable conformation of these inulin forms are important for inulin particles to remain intact long enough to bind and interact with immune cells. Hence, when suspensions of inulin particles are heated to high temperature so as to dissociate and solubilize the inulin particles, the resulting inulin solution loses all immunological and vaccine adjuvant activity. Inulin particles share properties relevant to their adjuvant action including the ability to enhance antigen processing and presentation by appropriate immune cells, properties not shared by more soluble inulin formulations.

Without wishing to be bound by theory, we have observed that the immune effects of each inulin polymorphic form increases in series as its temperature of solubility increases, such that particles of dIN are more temperature stable and adjuvant potent than gIN, and particles of eIN are in turn more temperature stable and adjuvant potent than particles of dIN. Thus, gIN, dIN or eIN form are progressively more adjuvant active. As disclosed by WO 87/02679, WO 2006/024100, and WO2011/032229, stable inulin formulations comprising gIN, dIN or eIN particles of appropriate size and composition are able to enhance humoral and/or cellular adaptive immune responses to co-administered vaccine antigens.

As described further in the present application, when studying the biological effects of inulin particles, we have now made the surprising finding that anti-inflammatory effects are also provided. More specifically, it has been found that, when cultured with human peripheral blood mononuclear cells (PBMC) or mouse splenocytes, inulin particles will upregulate rather than down-regulate expression of anti-inflammatory genes. Conversely, they will downregulate the expression of many pro-inflammatory genes and, in particular, inulin particles did not activate NFκB expression.

This was a highly surprising finding as it appears to contradict the widely accepted ‘danger model’ whereby all adjuvants are thought to work via activation of pro-inflammatory innate immune pathways through activation of NFκB and/or the inflammasome and thereby induce production of inflammatory cytokines such as TNF-a and IL-1. The danger model was largely developed based on the known adjuvant action of PAMPs, for example TLR agonists that activate the innate immune system but also directly or indirectly increase adaptive immune responses to co-administered antigens. PAMP-derived adjuvants all share the property that they induce pro-inflammatory cytokines including tumor necrosis factor (TNF)-a, interleukin (IL)-1, and IL-6 production. PAMPs induce these cytokines through activation of NFκB, a master transcription factor that induces inflammation in immune cells. Similarly, alum adjuvants and oil emulsion adjuvants activate the inflammasome, a tissue damage sensing mechanism which when activated also leads to the production of inflammatory cytokines including IL-1. By contrast, inulin particles when incubated with human PBMC, surprisingly do not activate NFκB but instead downregulate pro-inflammatory gene expression including interleukin (IL)-1, IL1RAP, IL18RAP, cyclooxygenase (Cox)-2, NALP3, NLRP3, NLRP12, CARD12, IFIT1, IFIT2, IFIT3, IDO, CXCL5, CXCL6, CXCR7, CD14, TLR4, NOD2, formyl receptors 1, 2 and 3, and upregulate genes associated with downregulation of innate immune responses and with inhibition of the pro-inflammatory IL1 cytokine pathway, including IL-1 receptor antagonist (IL-1RA), IL1RN, and IL1R2 as well as IL18BP, CD33, ATF3, TREM1, PPAR-gamma, FCRL2 and CD36. This data indicated that inulin particles have anti-inflammatory activity, leading to the first aspect of the current technology, as discussed below. The ability of inulin particles to inhibit inflammation was thus tested herein, with a view to potential use of inulin particles to treat or prevent inflammatory disease.

To test whether inulin particles could reduce the side effects of pro-inflammatory immune activators and adjuvant formulations, inulin particles were tested, in vitro and in vivo, with a range of PAMPs and innate immune activators including a broad range of TLR agonists, with the expectation that the inulin particles would inhibit both the inflammation and also inhibit the adjuvant activity induced by the PAMPs and other innate immune activators. The results were unexpected and surprising and led to the second aspect of the current technology. As predicted, the co-administration of inulin particles together with a classical PAMP innate immune activator such as CpG-motif containing oligonucleotides (ODN), down-modulated the inflammatory gene activation mediated by the CpG ODN. What was unexpected, however, was that, paradoxically, despite successfully inhibiting the inflammatory signals induced by the PAMP, the inulin particles actually enhanced the adjuvant activity of the PAMP on an adaptive immune response as measured by their ability to increase the protective memory immune response against a co-administered antigen. This finding was surprising given that the inulin particles were predicted to downregulate the pro-inflammatory ‘danger signals’ and innate immune activation induced by the co-administered PAMPs. Under the prevailing danger signal model of adjuvant action, inulin particles, by inhibiting inflammatory responses, would have been expected to reduce the PAMP adjuvant activity.

This experiment was subsequently repeated with a wide variety of further PAMP adjuvants, and the same beneficial effects of inulin particles were consistently observed—that is, reduction in inflammation yet enhanced adjuvant activity. In view of the previous lack of predictability in the art when combining adjuvants in a single composition, the consistent results obtained when combining inulin particles with all tested PAMPs was a further unexpected result. Without wishing to be restricted by theory, the downregulation by inulin particles of pro-inflammatory innate immune pathways induced by PAMPs, may paradoxically enhance the ability of the PAMPs to stimulate an adaptive immune memory response, suggesting that pro-inflammatory innate immune cytokines such as IL1 induced by PAMPs may, particularly if their levels are too high, suppress rather than stimulate an adaptive immune memory response. Thus, co-administration of inulin particles and an innate immune activator such as a PAMP together with a vaccine antigen, results in a surprisingly synergistic enhancement of the immune memory response against a co-administered vaccine antigen. The co-administration of inulin particles with an innate immune activator or PAMP also provided a surprising dose-sparing effect on the innate immune activator, such that the same adjuvant effect could be obtained with a reduced dose of the PAMP innate immune activator. Again this effect of inulin particles would not be predicted by the danger model of adjuvant action. This provides the opportunity to use inulin particles to achieve the same adaptive immune enhancement effect with a lower dose of the innate immune activator, thereby offering the opportunity to reduce dose-limiting side effects such as inflammation associated with innate immune activators including PAMPs. Co-administration of the inulin particles has further potential to reduce adverse inflammation-associated side effects of innate immune activators and PAMPs by blocking or attenuating inflammatory gene expression.

The applicants have found, therefore, that the combination of an inulin particle together with a PAMP innate immune activator results in a surprisingly favorable and synergistic immune response.

### SUMMARY OF THE DISCLOSED TECHNOLOGY

In certain embodiments, the present technology is directed to: a vaccine composition comprising: (a) inulin particles; (b) a pathogen-associated molecular pattern (PAMP); and (c) an antigen containing a protein or peptide derived from a neuronal self-antigen.

In certain embodiments, the present technology provides methods of preventing or treating a degenerative neurological disease in a subject, methods of vaccinating a subject against a neurodegenerative disease, and methods of manufacturing a vaccine according to the compositions herein.

In certain embodiments, the present technology relates to products and methods of inducing a favorable therapeutically potent immune response in patients with neurodegenerative diseases, such as AD, PD, LBD, etc. This is based on the surprising discovery that inulin particles combined with vaccine based on neuronal self-antigens can be used to induce adaptive and innate immune responses that are much stronger and transcend all types of immune responses generated with all known anti-AD/PD/DLB, etc. vaccines formulated in any known human adjuvants.

Further embodiments of the technology are based on the unexpected finding that the co-administration of inulin particles with an innate immune activator results in a favorable and synergistic modulation of the balance between innate and adaptive immune responses, such that, in various embodiments, a favorable anti-inflammatory and/or immune response, or an enhanced immune memory response, is achieved to a co-administered neuronal self-antigen with, if anything, a reduction of inflammation-associated side effects.

Accordingly, in certain embodiments the present technology provides a composition comprising inulin particles and vaccine targeting neuronal self-antigen for treating or preventing neurodegenerative disease, in a subject.

In certain embodiments, the present technology provides methods of treating or preventing neurodegenerative diseases in a subject. In certain embodiments, this can be accomplished without inflammation-associated side-effects, the method comprising the administration of a therapeutically-effective amount of a composition comprising inulin particles and vaccine targeting neuronal self-antigen to the subject.

In certain embodiments, the present technology provides for the use of a composition comprising various types of inulin particles and vaccine targeting neuronal self-antigen in the manufacture of a medicament for treating or preventing neurodegenerative disease, in a subject. A further embodiment is based on the unexpected finding that the co-administration of inulin particles with an innate immune activator results in a favorable and synergistic modulation of the balance between innate and adaptive immune responses, such that an enhanced immune memory response is achieved to a co-administered antigen with, if anything, a reduction of inflammation-associated side effects.

Accordingly, in certain embodiments, the technology provides a composition comprising inulin particles for use in the reduction or inhibition of inflammation, and/or for treating or preventing inflammatory disease, in a subject—for example, a method of reducing or inhibiting inflammation, or methods of treating or preventing (including prophylaxis against) inflammatory disease, in a subject, the methods comprising the administration of a therapeutically-effective amount of a composition comprising inulin particles to the subject; or use of a composition comprising inulin particles in the manufacture of a medicament of reducing or inhibiting inflammation, or of treating or preventing inflammatory disease, in a subject.

In certain embodiments, the reduction or inhibition of inflammation, or the treatment or prevention of inflammatory disease, is characterized by up-regulation of the expression of one or more anti-inflammatory genes and/or proteins and/or for the down-regulation of the expression of one or more pro-inflammatory genes and/or proteins in the subject, or optionally, specifically in the subject's myeloid or lymphoid cells including monocytes, dendritic cells, granulocytes, NK cells and/or lymphocytes. Exemplary pro-inflammatory genes for down-regulation in the subject in this context include interleukin (IL)-1, IL1RAP, IL18RAP, IL6, cyclooxygenase (Cox)-2, FPR2, MYD88, NALP3, NLRP3, NLRP12, CARD12, IFIT1, IFIT2, IFIT3, IDO, CXCL5, CXCL6, CXCR7, CD14, TLR4, NOD2, formyl receptors 1, 2 or 3, and members of CXCL chemokine family and/or TLR family members. Exemplary anti-inflammatory genes for upregulation in the subject in this context include IL-1 receptor antagonist (IL-1RA), IL1RN, and IL1R2, IL18BP, CD5L, CD33, ATF3, TREM1, PPAR-gamma, FCRL2 and CD36.

Accordingly, in certain embodiments, particles can be used to reduce or inhibit inflammation in a subject Inflammation in a subject may be caused, for example, by the exposure to one or more pro-inflammatory substances, including pathogenic infections including bacterial, viral, fungal or protozoal infection; exemplary infections including pandemic or seasonal influenza, inhalational anthrax, gram negative septicemia, systemic viraemia, encephalitis, Q fever, tularemia, small pox, chronic hepatitis B or C infection, SARS, pertussis, malaria, HIV, tuberculosis, polio, rabies, respiratory syncytial virus (RSV), shigella, mononucleosis, cytomegalovirus and toxic shock syndrome, allergenic substances; exemplary allergens being insect venom, cat or dog dander, rye grass, dust mite antigen, and pollens, or other pro-inflammatory substances or compositions, including, for example, compositions comprising pro-inflammatory substances, such as vaccine compositions or allergen-desensitization compositions, or anti-cancer treatments. In certain embodiments the inulin particles can be administered to the subject before, simultaneously with, or after the subject's exposure to the one or more pro-inflammatory substances.

In certain embodiments, the technology is directed to the use of inulin particles to reduce or inhibit inflammation in a subject that is caused by exposure (such as the administration of) a pro-inflammatory substance or composition that contains a substance comprising an innate immune activator and in particular a pathogen-associated molecular pattern (PAMP) including functional variants, derivatives or analogs thereof. The pro-inflammatory composition can, for example, be a pharmaceutically acceptable composition comprising a pro-inflammatory component that is intentionally administered to the subject, or a pro-inflammatory substance (e.g., biological or pathogenic substance or organism) to which the subject is intentionally or accidentally exposed. In this context, administration of the inulin particles to the subject before, or simultaneously with (including as a single mixture with), administration of or exposure to the pro-inflammatory composition can be most beneficial in certain embodiments. Thus, the composition comprising inulin particles can be used to reduce or inhibit the inflammatory response of the subject to the pro-inflammatory substance or composition.

In embodiments where the pro-inflammatory composition is an adjuvant composition that comprises PAMP, the inulin particles can be used to reduce, inhibit or prevent, one or more of a subject's adverse reactions to the PAMP, such as one or more adverse reactions including but not limited to: headache, fatigue, myalgia, diarrhea, fever, inflammation and granuloma formation at the site of injection, pyrogenicity, nausea, adjuvant arthritis, uveitis, eosinophilia, allergy, anaphylaxis, organ specific toxicity or immunotoxicity, i.e., the liberation of toxic quantities of inflammatory cytokines.

In certain embodiments, inulin particles can also be used in accordance with the previously discussed embodiments to treat or prevent inflammatory disease in a subject. Types of inflammatory diseases of particular interest for treatment or prevention in this context include, e.g., inflammatory diseases that are characterized by, or associated with NFkB activation, elevated IL-1 gene or protein levels or signaling, or IL-1 dysregulation. Exemplary inflammatory diseases include but are not limited to: migraine, chronic fatigue syndrome, rheumatoid arthritis, asthma, chronic obstructive airways disease, inflammatory bowel disease including ulcerative colitis and Crohn's disease, chronic fatigue syndrome, cryopyrin-associated periodic syndromes including neonatal onset multisystem inflammatory disease and Muckle Wells syndrome, inflammasome-associated disorders, psoriasis, atherosclerosis, type 1 or type 2 diabetes mellitus, hereditary fever syndromes, tumor necrosis factor receptor-associated periodic syndrome, Schnitzler syndrome, systemic lupus erythematosis, autoimmune hepatitis, Behçet disease and idiopathic recurrent pericarditis.

Accordingly, subjects for treatment by the methods herein can include those who have been, will be (in the sense that they are scheduled to be, or are at increased risk of being, in various embodiments within the following month, week, 6, 5, 4, 3, 2 or 1 days, or less than 24, 12, 6, 5, 4, 3, 2 or 1 hours), or are simultaneously being, exposed to one or more pro-inflammatory substances, including pathogenic infections (including bacterial, viral, fungal or protozoal infection), allergenic substances, or other pro-inflammatory compositions, including, for example, compositions comprising pro-inflammatory adjuvant, such as vaccine compositions or allergen-desensitization compositions; those suffering from or determined to be at risk of suffering from an inflammatory disease, including an inflammatory disease that is characterized by, or associated with, elevated IL-1 levels or signaling; or IL-1 dysregulation, e.g., migraine, chronic fatigue syndrome, rheumatoid arthritis, inflammatory bowel disease including ulcerative colitis and Crohn's disease, chronic fatigue syndrome, cryopyrin-associated periodic syndromes including neonatal onset multisystem inflammatory disease and Muckle Wells syndrome, inflammasome-associated disorders, psoriasis, atherosclerosis, type 2 diabetes, hereditary fever syndromes, tumor necrosis factor receptor-associated periodic syndrome, Schnitzler syndrome, Behçet disease and idiopathic recurrent pericarditis.

In other embodiments, the present technology provides immunological or pharmaceutically acceptable compositions comprising: (a) an anti-inflammatory component, such as inulin particles or one or more other anti-inflammatory inhibitors of IL-1 or one or more other anti-inflammatory inhibitors of NFκB activation; (b) a substance comprising one or more species of pathogen-associated molecular pattern (PAMP); and optionally, further comprising (c) one or more additional substances, for example, an antibody, antisense oligonucleotide, protein, antigen, allergen, a polynucleotide molecule, recombinant viral vector, a whole microorganism, or a whole virus.

In certain embodiments, pathogen-associated molecular patterns (PAMPs), as discussed herein, refers to molecules having the ability to activate the innate immune system. PAMPs can be directly or indirectly recognized by one or more innate immune receptors, or activate inflammatory gene pathways in immune cells. PAMPs can induce pro-inflammatory gene expression and protein production by immune cells including, for example, one or more of lymphocytes, monocytes, granulocytes, NK cells, dendritic cells, pro-inflammatory gene expression including, for example, one or more cytokines including TNF-α, G-CSF, GM-CSF, IL-1 through to IL-33 and more particularly IL-1, IL-4, IL-5, IL-6, IL-12, IL-13, IL-18, IL-20, interferons including type 1 interferons and gamma interferon, chemokines including the CXC family of chemokines including CXCL1 to CXCL17, CC family chemokines including CCL1 to CCL28, CX3C chemokines including fractalkine, C Family chemokines including XCL1 to XCL2, with induction of these pro-inflammatory genes typically involving activation of the NFκB transcription factor.

As used herein, the term “PAMP” includes not only those PAMPs found in nature, but also functionally equivalent mimetics, variants, derivatives and analogs thereof, including synthetic PAMPs. Numerous naturally-occurring and synthetic PAMPs are known in the art, many of which are discussed in more detail below.

In certain embodiments, component (a) of the composition above is an anti-inflammatory component, such as an anti-inflammatory inhibitor of IL-1 or anti-inflammatory inhibitor of NFkB. In certain embodiments, the anti-inflammatory component comprises inulin particles. Other anti-inflammatory inhibitors of IL-1 of particular interest are functionally-equivalent to inulin particles, in the sense of possessing an essentially equivalent anti-inflammatory property, activity or specificity or possessing an essentially equivalent adjuvant property. These can include one or more of IL1 receptor antagonists, IL1RA, Anakinra, Rilonacept, IL-1R/IL1RacP/Fc-fusion protein, Canakinumab, mass IL-1β blocking antibody, IL1 receptor blockers, IL-1RII, indomethacin, non-steroidal anti-inflammatory drugs (NSAID) including indomethacin, glucocorticoids, caspase inhibitors including caspase 1 inhibitors, inflammasome inhibitors, chloroquine, P2X7 receptor inhibitors, ST2 receptor inhibitors, curcumin, resveratrol, and eicosanoid biosynthesis inhibitors.

In certain embodiments, component (b) of the composition above is a substance comprising one or more pathogen-associated molecular patterns (PAMP). In certain embodiments, the substance comprises no greater than ten distinct molecular species of PAMP, e.g., nine or less, eight or less, seven or less, six or less, five or less, four or less, three or less, two or less, or only one distinct molecular species of PAMP. In certain embodiments, the limitation on the number of distinct molecular species of PAMP in component (b) can be applied only in respect of combination with inulin particles comprising a specific type of inulin.

Thus, for example, in various embodiments, component (b) comprises no greater than ten, nine, eight, seven, six, five, four three, two or one distinct molecular species of PAMP where the inulin particles in component (a) comprise gamma inulin, or delta inulin, or epsilon inulin.

In certain embodiments, distinct molecular species of PAMP can be structurally distinct. Such a structural distinction can, for example, be determined by known methods of structural analysis, such as mass spectroscopy, nuclear magnetic resonance, FTIR, circular dichroism, or differential scanning calorimetry.

In certain embodiments, distinct molecular species of PAMP can be functionally distinct. Functionally distinct molecular species of PAMP can be characterized by displaying a different binding profile to innate immune receptors. This can be assessed, for example, by measuring the binding of PAMP species to a panel of innate immune receptors which can, for example, comprise receptors selected from TLRs, such as human or animal TLR-1, TLR-2, TLR-3, TLR-4, TLR-5, TLR-6, TLR-7, TLR-8, TLR-9, murine TLR-11; NOD-1, NOD-2, other NOD-like receptors (NLRs) such as NLRP1, NLRP3, NLRP12, NLRC4; DECTIN-1; DC-SIGN; AIM-2; C-type Lectin, MD2; CD14; LBP; CD36; RIG-I-like receptors including RIG-I, MDA5, LGP2 and/or ASC. Binding of PAMP species to these receptors can be assessed by routine methods, such as surface plasmon resonance. The skilled person will be able to determine appropriate conditions under which to assess binding, which in certain embodiments can be selected to provide an assessment of binding specificity under moderate to highly stringent conditions. Additionally, or alternatively, as known by the skilled person, the property of a PAMP can be detected or quantified in an immune cell line such as the THP-1 or RAW cell line, by a functional assay, for example using an NFkB activation reporter assay such as the Thermo Scientific Pierce Luciferase Assay Kit or by measurement of inflammatory gene or protein activation in response to incubation of the cell line with the substance being tested for PAMP activity.

In various embodiments, the totality of PAMP present in the component (b) of the composition (and, optionally, all of the PAMP in the composition, in the event that component (c) contains further PAMP) will not bind to more than 20, 19, 18, 17, 16, 15, 14, 13, 12, 11, 10, 9, 8, 7, 6, 5, 4, 3, 2 or 1 of the receptors in the panel of innate immune receptors as described above.

Although capable of indirectly activating the innate immune system, through lysis of the lysosome in phagocytosing cells, inflammasome activation, caspase activation, induction of immune cell death, and release of endogenous DNA, antigen-binding carrier materials with adjuvant properties such as alum (or other metal salts or precipitates such as magnesium, calcium or aluminum phosphates, sulfates, hydroxides or hydrates thereof) have not been shown themselves to bind a specific PAMP receptor, and do not mimic a molecular pattern expressed by a pathogen and as they thereby act in a different manner to molecularly defined PAMPs that bind specific innate immune receptors, they do not form part of the definition of PAMPs as used in this application.

It will be appreciated that in certain embodiments, optional component (c) of the compositions above can, for example, include one or more additional substances including but not limited to: an antibody, antisense oligonucleotide, protein, antigen, allergen, a polynucleotide molecule, recombinant viral vector, a whole microorganism, or a whole virus, and so component (c) may contribute one or more additional PAMPs to the composition. For example, whole microorganisms, whole viruses, endotoxin and the like will contain high numbers (certainly greater than ten) of molecularly, structurally, physically and/or functionally distinct molecular species of PAMP. Thus, in certain embodiments, the total number of distinct molecular species of PAMPs in the composition of the second aspect of the present technology can be greater than ten. But that does not detract from the requirement, in certain embodiments, that component (b) of the composition comprises no greater than ten or fewer distinct molecular species of PAMP. Typically, therefore, the substance(s) optionally present in component (c) will be molecularly, structurally and/or functionally different molecules to the molecules present in component (b).

The one or more PAMPs (in certain embodiments all PAMPs) present in component (b) of the compositions can possess a weight average molecular weight of up to but no more than 200,000 KDa, such as up to but no more than: 150,000 KDa, 100,000 KDa, 50,000 KDa, 40,000 KDa, 20,000 KDa, 10,000 KDa, 5,000 KDa, 2,000 KDa, 1,000 KDa, 500 KDa, 450 KDa, 400 KDa, 350 KDa, 300 KDa, 250 KDa, 200 KDa, 150 KDa, 100 KDa, 50 KDa, 40 KDa, 30 KDa, 20 KDa, 10 KDa, 9 KDa, 8 KDa, 7 KDa, 6 KDa, 5 KDa, 4 KDa, 3 KDa, 2 KDa, or 1 KDa or less.

In certain embodiments, a composition herein can be a pharmaceutically acceptable composition. As used herein, a “pharmaceutically acceptable composition” refers to a composition that is safe for administration to a subject, such as a human subject, by injection, such as intravenous, subcutaneous or intramuscular injection. In one embodiment, the composition is defined as being safe if it contains no, or substantially no, endotoxin. Endotoxin is often used synonymously with the term lipopolysaccharide, which is a major constituent of the outer cell wall of Gram-negative bacteria. It includes a polysaccharide (sugar) chain and a lipid moiety, known as lipid A, which is responsible for the toxic effects observed with endotoxin. The polysaccharide chain is highly variable among different bacteria and determines the serotype of the endotoxin and the lipid components are also highly variable such that a single endotoxin sample may contain 10's to 100's of distinct molecular species. Endotoxin is approximately 10 kDa in size but can form large aggregates up to 1000 kDa. Endotoxin is typically harmful and pyrogenic in therapeutic compositions and regulatory authorities have imposed strict limitations on the allowable levels of endotoxin within a pharmaceutical composition. Accordingly, the level of endotoxin in a composition according to certain embodiments herein should be minimized and may be, in various embodiments, less than 100 endotoxin units (EU) per dose, such as less than 90, 80, 70, 60, 50 40, 30, 20, 10, 5, 4, 3, 2, 1 or less EU per dose. In various embodiments, the concentration of endotoxin in a composition herein is less than 200 EU/m3, such as less than 150, 100, 90, 80, 70, 60, 50 40, 30, 20, 10, 5, 4, 3, 2, 1 or less EU/m3. In some embodiments, these limitations may be applied to the compositions herein where the inulin particles present in component (a) comprise just one or two of gamma inulin, delta inulin or epsilon inulin. Methods of measuring endotoxin levels, such as the limulus amoebocyte assay (LAL) method, are well known in the art.

In certain embodiments, a composition herein can optionally be packaged and/or presented in a convenient or unit dosage form.

The amount or concentration of PAMP present in component (b) of the compositions herein (and, optionally, the amount or concentration of PAMP present in the entire composition) is, in certain embodiments, less than the amount of PAMP required in an equivalent composition that differs only in that it does not include the inulin particles (or other equivalent anti-inflammatory component). In other words, the presence of inulin particles (or other equivalent anti-inflammatory component) in component (a) of the compositions herein can, in certain embodiments, provide a composition that is able to induce or modulate an immune response in a subject using less PAMP in component (b) than would be required to achieve the same level or type of induction or modulation compared to an equivalent composition that differs only in that it does not include the inulin particles (or other equivalent anti-inflammatory component).

Accordingly, in certain embodiments, the amount or concentration of the one or more PAMPs in component (b) of the composition (and, optionally, the amount and/or concentration of the one or more PAMPs present in the entire composition) can be less than, e.g., less than 90%, 80%, 70%, 60%, 50%, 40%, 30%, 20%, 10%, 5%, 4%, 3%, 2%, 1%, 0.5%, 0.4%, 0.3%, 0.2%, 0.1%, 0.05%, 0.04%, 0.02%, 0.01% or less (by weight) than the optimal amount of the same one or more PAMPs that is required in an equivalent composition that differs only in that it does not include the inulin particles (or other equivalent anti-inflammatory component). In certain embodiments, the optimal amount of PAMPs in the equivalent composition is the amount that is required to achieve the desired effect of induction or modulation of an immune response including for example adjuvant enhancement of an immune response to a co-administered antigen without being so high as to cause unacceptable levels of inflammatory and/or other side-effects. This can be determined empirically for each PAMP using routine methods, for example by performing dose-ranging toxicity studies in animal models, or by use of surrogate measures such as the extent of NFkB activation in cell-based functional assays.

Indeed, such an equivalent composition can be entirely incapable of achieving the same level or type of induction or modulation, no matter how much PAMP is included, in the absence of inulin particles. In some embodiments, these limitations can be applied to the composition of the second aspect of the technology where the inulin particles present in component (a) comprise just one or two of gamma inulin, delta inulin or epsilon inulin.

In certain embodiments, a suitable or optimal ratio of inulin particles (or other equivalent anti-inflammatory component) in component (a) to PAMP in component (b) of the composition, in order to achieve a desired effect, can be determined empirically by the skilled person for each specific combination of inulin particles and PAMP using routine methods. In certain embodiments, however, the weight/weight ratio of inulin particles (or other equivalent anti-inflammatory component) to PAMP is in the range of from 10,000:1 to 1:1, from 1000:1 to 1:1, from 100:1 to 1:1, or from 100:1 to 10:1.

Accordingly, an immunological composition according to certain embodiments herein can include an effective amount for inducing a desired immune response of a combination of components, wherein the combination includes at least one inulin particle (or other equivalent anti-inflammatory component) and at least one PAMP innate immune activator. The PAMP innate immune activator in the immunological composition can be of any type of PAMP innate immune activator known in the art. For example, the PAMP innate immune activator can be one or more of any of the group of substances that are known agonists of innate immune receptors. Accordingly, a PAMP innate immune activator for use in the present technology can bind and be an agonist of any one or more innate immune receptors of, TLRs, RNA helicases, NOD1, NOD2, other NOD-like receptors (NLRs) such as NLRP1, NLRP3, NLRP12, NLRC4; DECTIN-1; DC-SIGN; AIM-2; C-type Lectin, MD2; CD14; LBP; RIG-I-like receptors including RIG-I, MDA5, LGP2 and/or ASC, C-type lectin receptors, complement receptors, Fc receptors, and scavenger receptors.

In another embodiment, the present technology provides a kit of parts comprising: (a) a first container that contains a composition comprising an anti-inflammatory component, such particles of inulin and/or one or more other anti-inflammatory inhibitors of IL-1 or NFkB (as discussed above); and (b) a second container that contains a substance comprising a PAMP.

Thus, in certain embodiments, the substance present in the second container comprises no greater than ten distinct molecular species of PAMP, e.g., nine or less, eight or less, seven or less, six or less, five or less, four or less, three or less, two or less, or only one distinct molecular species of PAMP. In certain embodiments, the limitation on the number of distinct molecular species of PAMP in the substance present in the second container can be applied only in respect of kits in which the first container contains a composition comprising particles of a specific type of inulin, such as only gamma inulin, only delta inulin or only epsilon inulin.

In another embodiment, the totality of PAMP that is present in the second container may not bind to more than 20, 19, 18, 17, 16, 15, 14, 13, 12, 11, 10, 9, 8, 7, 6, 5, 4, 3, 2 or 1 of the receptors in the panel of innate immune receptors as described above.

In certain embodiments, either or both of the first container and second container in the kit can optionally further comprise one or more additional substances, for example, one or more of an antibody, antisense oligonucleotide, protein, antigen, allergen, a polynucleotide molecule, recombinant viral vector, a whole microorganism, or a whole virus.

In various embodiments, the one or more PAMPs (in certain embodiments all PAMPs) present in the second container of the kit can possess a weight average molecular weight of up to but no more than 200,000 KDa, such up to but no more than: 150,000 KDa, 100,000 KDa, 50,000 KDa, 40,000 KDa, 20,000 KDa, 10,000 KDa, 5,000 KDa, 2,000 KDa, 1,000 KDa, 500 KDa, 450 KDa, 400 KDa, 350 KDa, 300 KDa, 250 KDa, 200 KDa, 150 KDa, 100 KDa, 50 KDa, 40 KDa, 30 KDa, 20 KDa, 10 KDa, 9 KDa, 8 KDa, 7 KDa, 6 KDa, 5 KDa, 4 KDa, 3 KDa, 2 KDa, 1 KDa or less.

In certain embodiments, either or both of the first container or second container in the kit of the third aspect of the present technology contains a unit dose of the material contained therein.

In various embodiments, either or both of the first container or second container in the kit is a pharmaceutically acceptable composition, as defined above. Accordingly, in various embodiments, the level of endotoxin in either or both of the first container or second container in the kit can be less than 100 EU per dose, such as less than 90, 80, 70, 60, 50 40, 30, 20, 10, 5, 4, 3, 2, 1 or less EU per dose. The concentration of endotoxin in either or both of the first container or second container in the kit can be less than 200 EU/m3, such as less than 150, 100, 90, 80, 70, 60, 50 40, 30, 20, 10, 5, 4, 3, 2, 1 or less EU/m3. In some embodiments, these limitations may be applied to the kit herein where inulin particles present in the first container comprise just one or two of gamma inulin, delta inulin or epsilon inulin.

In various embodiments, the amount or concentration of PAMP present in the second container of the kit is less than the optimal amount, such as less than 90%, 80%, 70%, 60%, 50%, 40%, 30%, 20%, 10%, 5%, 4%, 3%, 2%, 1%, 0.5%, 0.4%, 0.3%, 0.2%, 0.1%, 0.05%, 0.04%, 0.02%, 0.01% or less (by weight), of PAMP that is required, when used alone to achieve the desired level or type of induction or modulation of the immune response.

In certain embodiments the weight/weight ratio of inulin particles (or other equivalent anti-inflammatory component) in the first container to PAMP in the second container may be in the range of from 10000:1 to 1:1, from 1000:1 to 1:1, from 100:1 to 1:1, or from 100:1 to 10:1.

In a further embodiment, the substance comprising a PAMP can be an innate immune activator, and can comprise one or more a substances that binds and is an agonist of one or more of a TLR, RNA helicase, NOD1, NOD2, other NOD-like receptors (NLRs) such as NLRP1, NLRP3, NLRP12, NLRC4; DECTIN-1; DC-SIGN; AIM-2; C-type Lectin, MD2; CD14; LBP; RIG-I-like receptors including RIG-I, MDA5, LGP2 and/or ASC, C-type lectin receptor, complement receptor, Fc receptor, and scavenger receptor.

In a further embodiment, one or more PAMP can be a substance such as diacyl lipopeptide, triacyl lipopeptide, Pam3CSK4, lipoteichoic acid, peptidoglycan, HSP70, zymosan, ssRNA, dsRNA, dsDNA, poly(I:C), poly(I:C-LC), Hiltonol™, PolyI:PolyC12-U, Ampligen™ MPLA, heat shock protein, fibrinogen, heparan sulfate fragments, hyaluronic acid fragments, synthetic TLR4 agonist, imidazoquinoline, gardiquimod, loxoribine, bropirimine, CL264, R848, CL075 PolyU, imiquimod, resiquimod, ssPolyU/LyoVec, ssRNA40/LyoVec, unmethylated CpG oligonucleotide, Class B ODN, Class C ODN, CpG2006, CpG1826, CpG7909, C12-iE-DAP, iE-DAP, Tri-DAP, muramyl dipeptide (MDP), L18-MDP, M-TriDAP, murabutide, PGN-ECndi, PGN-ECndss, PGN-Sandi, porin, lipoarabinomannan, phospholipomannan, glucuronoxylomannan, glycosylphosphatidylinositol (GPI)-anchored protein, hemozoin, viral dsDNA, synthetic dsDNA, viral dsRNA, synthetic dsRNA peptidoglycan containing the muramyl dipeptide NAG-NAM-gamma-D-glutamyl-meso diaminopimelic acid, peptidoglycan containing the muramyl dipeptide NAG-NAM-L-alanyl-isoglutamine, N-formyl methionine, muramyl tripeptide, beta-1,3-glucan, zymosan, cord factor, trehalose-6,6-dibehenate, Poly(dA:dT), Poly(dG:dC), 5′ppp-dsRNA, low density lipoprotein (LDL), oxidized LDL, chemically modified LDL, hemozoin, ATP.

In a further embodiment, the inulin particle can comprise inulin including but not limited to: gamma inulin, delta inulin and epsilon inulin, or combinations of any one or more of these inulins; optionally with aluminum phosphate or aluminum hydroxide, including but not limited to: phosgammulin, phosdeltin, phosepsilin, algammulin, and algammulin, aldeltin or alepsilin. Alpha and/or beta inulin or other modified inulin particles can also be used in addition to, or instead of, gamma, delta or epsilon inulin, providing they are in a suitable particulate form.

In a further embodiment, the composition comprising inulin particles comprises particles of at least two inulin preparations, and the preparations can differ in the polymorphic form of the inulin present and/or the presence or species of an antigen-binding carrier material. For example, in various embodiments, the inulin particles can comprise—

gamma inulin (or a combination of gamma inulin with aluminum phosphate or aluminum hydroxide) mixed with delta inulin; or

gamma inulin (or a combination of gamma inulin with aluminum phosphate or aluminum hydroxide) mixed with epsilon inulin; or

delta inulin (or a combination of delta inulin with aluminum phosphate or aluminum hydroxide) mixed with gamma inulin; or

delta inulin (or a combination of delta inulin with aluminum phosphate or aluminum hydroxide) mixed with epsilon inulin; or

epsilon inulin (or a combination of epsilon inulin with aluminum phosphate or aluminum hydroxide) mixed with gamma inulin; or

epsilon inulin (or a combination of delta inulin with aluminum phosphate or aluminum hydroxide) mixed with delta inulin.

In the forgoing list, any recitation of gamma, delta or epsilon inulin can optionally also be replaced with alpha inulin or beta inulin.

In a further embodiment, the compositions herein can further comprise one or more additional substances, for example, an antibody, antisense oligonucleotide, protein, antigen, allergen, a polynucleotide molecule, recombinant viral vector, a whole microorganism, or a whole virus.

Accordingly, in a further embodiment, the composition can further comprise one or more antigens. The one or more antigens can be any type of antigen known in the art, including but not limited to: proteins, glycoproteins, peptides, polypeptides, cells, cell extracts, polysaccharides, polysaccharide conjugates, lipids, glycolipids, nucleic acids and carbohydrates, or conjugates of carbohydrates or lipids with protein, polypeptide/peptide antigens, peptide mimics of polysaccharides; antigens may also be encoded within nucleic acid sequences. In certain embodiments, antigens can be in a crude, purified or recombinant form. Antigens can be derived from an infectious pathogen such as a virus, bacterium, fungus or parasite, or the antigen may be derived from a tumor antigen, an allergen, or self-protein.

In the embodiments herein where one or more antigens, in particular one or more vaccine antigens is/are included, it can also be suitable to further include one or more antigen-binding agents in the same mixture as the one or more antigens.

In certain embodiments, the present technology also contemplates methods of preparing the compositions herein. In various embodiments, the methods can comprise the step of providing the component parts and then bringing them together to form a composition.

In certain embodiments, the present technology also contemplates methods of stimulating or modulating an immune response, including an antigen-specific immune response, in a subject by administering to the subject a therapeutically effective amount of an immunological compositions herein or using a kit herein. In various embodiments, the methods include the steps of administering to the subject the immunological composition or kit, wherein the composition, or each component, is administered in an effective amount and at an effective time and route for inducing a desired immune response or effect.

Accordingly, additional embodiments provides methods of inducing or modulating an immune response in a subject, wherein said methods comprise administering to the subject a therapeutically effective amount of the composition, or simultaneously, sequentially or separately administering therapeutically effective amounts of the contents of the first or second containers of a kit herein. Further embodiments provide a composition or kit for use in inducing or modulating an immune response in a subject; or the use of a composition or kit herein in the manufacture of a medicament for inducing or modulating an immune response in a subject.

In other embodiments, the modulation of the immune response can comprise increasing the speed of development of the immune response, compared to the speed of development of the immune response obtained in the subject with an equivalent composition that differs only in that it does not include the inulin particles. The immune response in question can be, for example, an adaptive immune response to one or more antigens. In various embodiments, the adaptive immune response can comprise a response from one or more of T-cells (including one or more of CD4+ and/or CD8+ T-cells) or B-cells, and can for example be determined with respect to the production of one or more types or subtypes of antibodies, such as any one or more of IgA, IgE, IgG1, IgG2a, IgG2b, IgG3, IgG4 or IgM or with respect to the production of one or more types of cytokines, such as any one or more of IFN-γ, TGF-β, GM-CSF, TNFα, IL-1, IL-2, IL4, IL-5, IL-6, IL7, IL-8, IL10, IL12, IL13, IL-17 or IL-20.

In other embodiments, the modulation of the immune response can comprise increasing the specificity of the subject's immune response, compared to the specificity of the immune response obtained in the subject with an equivalent composition that differs only in that it does not include the inulin particles. The immune response in question can be, for example, an adaptive immune response. In various embodiments, the adaptive immune response can comprise a response from one or more of T-cells (including one or more of CD4+ and/or CD8+ T-cells) or B-cells, and may for example be determined with respect to the production of one or more types or subtypes of antibodies, such as any one or more of IgA, IgE, IgG1, IgG2, IgG3, IgG4 or IgM. Increased specificity can, for example, include increasing the level of specificity of the B- or T-cell response to any antigen that is presented in the administered composition(s).

In other embodiments, the modulation of the immune response can comprise increasing the magnitude or increasing the duration of the subject's immune response, compared to the magnitude or duration respectively of the immune response obtained in the subject with an equivalent composition that differs only in that it does not include the inulin particles. The immune response in question can be, for example, an adaptive immune response. In various embodiments, the adaptive immune response can comprise a response from one or more of T-cells (including one or more of CD4+ and/or CD8+ T-cells) or B-cells, and can for example be determined with respect to the production of one or more types or subtypes of antibodies, such as any one or more of IgA, IgE, IgG1, IgG2, IgG3, IgG4 or IgM.

In other embodiments, the modulation of the immune response can comprise modifying the type of the subject's immune response, compared to the type of the immune response obtained in the subject with an equivalent composition that differs only in that it does not include the inulin particles. The type of immune response in question can be, for example, an adaptive immune response. In various embodiments, the type of adaptive immune response can be characterized by the speed, magnitude, specificity, or duration of one or more aspects of an adaptive immune response relative to other aspects of the adaptive response, including for example, the response from one or more of T-cells (including one or more of CD4+ and/or CD8+ T-cells; Th1, Th2, Th17 and Treg cells) and/or B-cells, and can for example be determined with respect to the production of one or more types or subtypes of antibodies compared to one or more other subtypes, such as any one or more of IgA, IgE, IgG1, IgG2, IgG3, IgG4 or IgM compared to any one or more of the others.

Other examples of modifying the type of the subject's immune response, in accordance with these embodiments, include modifying the balance between the innate and adaptive immune response; enhancing the immune memory response; altering the type of immune response such as by enhancing or inhibiting the Th1, Th2, Th17 or Treg response compared to the other responses; suppressing the IgE response; or enhancing one or more of the IgA, IgM or IgG subtype responses. Thus, in certain embodiments, the technology provides methods to obtain an optimal immune subclass or subtype response, including the optimal T- or B-cell response to a vaccine antigen, where it could not be achieved to the same extent using an equivalent composition or kit that differs only in that it does not include the inulin particles (or other equivalent anti-inflammatory component).

In other embodiments, the technology provides a method of inducing or modulating an immune response to an antigen, wherein said method comprises: administering to a subject a therapeutically effective amount of a composition herein, wherein said composition also comprises the antigen and, optionally, further comprises antigen-binding carrier material; or simultaneously, sequentially or separately administering to a subject therapeutically effective amounts of the contents of the first and second containers of a kit herein, wherein said contents of the first or second containers of the kit also comprises the antigen and, optionally, further comprises antigen-binding carrier material.

Thus, in certain embodiments, the technology provides a composition that comprises an antigen and, optionally, further comprises antigen-binding carrier material, for use in modulating an immune response to the antigen; and also provides for a kit, wherein the contents of the first or second containers of the kit also comprises an antigen and, optionally, further comprises antigen-binding carrier material, for use in inducing or modulating an immune response to the antigen. In certain embodiments, the composition also comprises an antigen and, optionally, further comprises antigen-binding carrier material, in the manufacture of a medicament for inducing or modulating an immune response to the antigen; and also provides for the use of a kit, wherein the contents of the first or second containers of the kit also comprises an antigen and, optionally, further comprises antigen-binding carrier material, for the manufacture of a medicament for inducing or modulating an immune response to the antigen.

In certain embodiments, the technology is directed to a method of vaccinating a subject, wherein said method comprises: administering to a subject a therapeutically effective amount of a composition according to the second aspect of the present technology, wherein said composition also comprises an antigen and, optionally, further comprises antigen-binding carrier material; or simultaneously, sequentially or separately administering to a subject therapeutically effective amounts of the contents of the first or second containers of a kit herein, wherein said contents of the first or second containers of the kit also comprises an antigen and, optionally, further comprises antigen-binding carrier material. Thus, in certain embodiments the technology provides a composition that comprises an antigen and, optionally, further comprises antigen-binding carrier material, for use in vaccinating a subject; and also provides for a kit, wherein the contents of the first or second containers of the kit also comprises an antigen and, optionally, further comprises antigen-binding carrier material, for use in the vaccinating a subject. In certain embodiments, the vaccinating of the subject is against a neurodegenerative disease.

In certain embodiments, the technology herein provides for the use of a composition that comprises an antigen and, optionally, further comprises antigen-binding carrier material, in the manufacture of a medicament for the vaccination of a subject; and also provides for the use of a kit, wherein the contents of the first or second containers of the kit also comprises an antigen and, optionally, further comprises antigen-binding carrier material, for the manufacture of a medicament for the vaccination of a subject.

Suitable vaccine antigens for use in accordance with certain embodiments herein can include any of those described elsewhere in this application. The amount or concentration of antigen used in certain embodiments herein can be less than the amount of antigen that is required in an equivalent composition or kit that differs only in that the composition or kit does not include inulin particles (or other equivalent anti-inflammatory component). In other words, the presence of inulin particles (or other equivalent anti-inflammatory component) in the compositions and/or kits can provide for methods and uses that can induce or modulate an immune response herein with less antigen.

Accordingly, in various embodiments, the amount or concentration of one or more antigens in the compositions or kits herein can be less, such as less than: 90%, 80%, 70%, 60%, 50%, 40%, 30%, 20%, 10%, 5%, 4%, 3%, 2%, 1%, 0.5%, 0.4%, 0.3%, 0.2%, 0.1%, 0.05%, 0.04%, 0.02%, 0.01% or less (by weight) than the optimal amount of the same one or more antigens that is/are required to achieve a corresponding desired immune response, or effective vaccination of a subject, in an equivalent composition or kit that differs only in that it does not include the inulin particles (or other equivalent anti-inflammatory component).

The optimal amount in the equivalent composition is the amount that is required to achieve the desired effect of induction or modulation of an immune response without being so high as to cause unacceptable levels of inflammatory or other side-effects. This can be determined empirically by the skilled person for each antigen and PAMP using routine methods. Indeed, in certain embodiments, such an equivalent composition may be entirely incapable of achieving the same level or type of immune induction or modulation, or vaccination, no matter how much antigen is included, in the absence of inulin particles (or other equivalent anti-inflammatory component).

In certain embodiments, the present technology also provides methods of down-modulating an existing unwanted immune response in a subject, such as an allergy to an allergen, or a chronic inflammatory condition, for example by downregulation of allergen-specific IgE or induction of blocking allergen-specific IgG. Such methods can include the steps of administering to the subject a composition herein, or the components of a kit herein, and optionally a further component such as an antigen or allergen wherein each component is administered in an effective amount and at an effective time and route for inhibiting or down-modulating the unwanted immune response and/or inducing a favorable counter-regulatory immune response.

In certain embodiments, the present technology provides methods for the allergen desensitization of a subject, wherein said method comprises: administering to a subject a therapeutically effective amount of a composition herein, wherein said composition also comprises an allergen and, optionally, further comprises allergen-binding carrier material; or simultaneously, sequentially or separately administering to a subject therapeutically effective amounts of the contents of the first and second containers of a kit herein, wherein said contents of the first or second containers of the kit also comprises an allergen and, optionally, further comprises an allergen-binding carrier material. That is, in certain embodiments, a composition also comprises an allergen and, optionally, further comprises allergen-binding carrier material, for use in the allergen desensitization of a subject; and also provides for a kit herein, wherein the contents of the first or second containers of the kit also comprises an allergen and, optionally, further comprises allergen-binding carrier material, for use in the allergen desensitization of a subject. Such embodiments can provide for the use of a composition that comprises an allergen and, optionally, further comprises allergen-binding carrier material, in the manufacture of a medicament for the allergen desensitization of a subject; and also provides for the use of a kit herein, wherein the contents of the first and/or second containers of the kit also comprises an allergen and, optionally, further comprises allergen-binding carrier material, for the manufacture of a medicament for the allergen desensitization of a subject.

In certain embodiments, the present technology provides methods of treating cancer, wherein said method comprises administering to a subject a therapeutically effective amount of a composition herein; or simultaneously, sequentially or separately administering to a subject therapeutically effective amounts of the contents of the first and second containers of a kit herein. Thus, certain embodiments provide a composition or kit for use in the treatment of cancer; or the use of a composition or kit herein in the manufacture of a medicament for the treatment of cancer.

In certain embodiments, a composition or the contents of the first or second containers of a kit herein further comprises a cancer antigen.

In other embodiments, the present technology provides a method of manufacturing a vaccine, the method comprising the step of combining an antigen, and optionally also an antigen-binding carrier material, with one or more components (for example, components (a) and (b)) of a composition herein, thereby to produce a vaccine composition. In certain embodiments, the technology provides for the use of a composition herein as an adjuvant in a vaccine.

In the examples and embodiments discussed herein, it is demonstrated that the compositions according to certain embodiments of the present technology can provide single vaccine dose protection against an otherwise lethal condition. Also, compositions of the certain embodiments, when formulated as a vaccine against influenza, can provide effective single dose protection in a murine model. Single dose vaccine protection is extremely desirable and, hitherto, hard to achieve in the field of vaccinology. Yet the compositions of certain embodiments herein have been found to provide single dose vaccine protection

Accordingly, in certain embodiments, the present technology provides a single-dose vaccine composition comprising inulin particles (optionally in the form of a kit), an antigen and, optionally, an antigen-binding carrier material. Such a single dose vaccine composition is effective to provide vaccine protection in the subject with only a single administration of a dose of the vaccine.

In certain embodiments, the present technology provides a method of vaccinating a subject the method comprising administering to the subject a dose of a vaccine herein, in certain embodiments a single does. In various embodiments, the method can comprise one or more additional steps, or can comprise no additional steps of administering the vaccine after the initial administration.

In certain embodiments, the present technology provides a single-dose of the vaccine as defined above for use in vaccinating a subject by a method comprising administering to the subject a single-dose of the vaccine; or the use of a single-dose of the vaccine as defined above for the manufacture of a medicament for use in vaccinating a subject by a method comprising administering to the subject a single-dose of the vaccine.

A further advantageous feature of the present embodiments is that the compositions, substances, kits and methods described herein are particularly effective in treating those subject groups that may typically fail to respond at all, or adequately, to conventional adjuvant and vaccine compositions. Such subject groups may include the young, the older population and pregnant women. In some embodiments, influenza vaccines of the present technology may be of particular interest for administration to such subjects.

Accordingly, in various embodiments the subject to be treated by the compositions, substances, kits and methods herein can be child, for example a male or female child. The child can be, for example, less than 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, 3 years old, 2 years old, 1 year old, 11 months old, 10 months old, 9 months old, 8 months old, 7 months old, 6 months old, 5 months old, 4 months old, 3 months old, 2 months old, or 1 month old, relative to the date of their birth.

In other embodiments, the subject to be treated by the compositions, substances, kits and methods according to the other aspects of the present technology may be an older human, for example a male or female. The older human can be, for example, at least 40 years old, at least 45 years old, at least 50 years old, at least 55 years old, at least 60 years old, at least 65 years old, at least 70 years old, at least 75 years old, at least 80 years old, at least 85 years old, or at least 90 years old.

In certain embodiments, the subject to be treated by the compositions, substances, kits and methods according to the other aspects of the present technology can be a pregnant female. The female can be up to, or at least, 5, 10, 15, 20, 25, 30, 35 or 40 weeks pregnant.

In other embodiments, the technology herein provides a method of identifying optimal concentrations and ratio of components (a) and (b) of a composition herein, the method comprising the optional step of combining an antigen, and optionally also an antigen-binding carrier material, with components (a) and (b) of the composition, administering the combined composition in a range of different doses to a series of subjects and then measuring the resulting immune response and optionally challenging the subject with a live pathogen thereby allowing the optimal composition to be identified.

In certain embodiments, the contents of the first or second containers of a kit herein form, optionally with an antigen, an assay kit for identification of the optimal composition for a desired immune application.

In another embodiment a method of manufacturing an assay kit is provided, the method comprising the step of combining an antigen, and optionally also an antigen-binding carrier material, with components (a) and (b) of a composition herein, thereby to produce a vaccine assay kit.

In various embodiments, the compositions disclosed herein comprise at least one immunogen, wherein each at least one immunogen comprises a region A coupled to a region B; wherein region A comprises at least one amyloid-β (Aβ) B cell epitope or at least one Tau B cell epitope or at least one α-synuclein B cell epitope or a combination of at least one amyloid-β (Aβ) B cell epitope and at least one Tau B cell epitope or a combination of at least one amyloid-β (Aβ) B cell epitope and at least one α-synuclein B cell epitopes, or a combination of at least one Tau B cell epitope and at least one α-synuclein B cell epitope, or a combination of at least one amyloid-β (Aβ) B cell epitope and at least one Tau B cell epitope and at least one α-synuclein B cell epitope, and region B comprises a plurality of foreign T helper cell (Th) epitopes. In another aspect, the composition comprises at least two immunogens, wherein each immunogen is distinct.

In some embodiments, the immunogen comprises a linker domain between region A and region B. In other embodiments, the immunogen comprises linker domains between each epitope. In some embodiments, the order of the regions is A-B and in other embodiments, the order is B-A. In some embodiments, the compositions further comprise an adjuvant or a pharmaceutical excipient or both.

In other embodiments, the composition comprises at least one nucleic acid molecule encoding an immunogen, wherein the immunogen comprises at least one amyloid-β (Aβ) B cell epitope or at least one Tau B cell epitope or at least one α-synuclein B cell epitope or a combination of at least one amyloid-β (Aβ) B cell epitope and at least one Tau B cell epitope or a combination of at least one amyloid-β (Aβ) B cell epitope and at least one α-synuclein B cell epitopes, or a combination of at least one Tau B cell epitope and at least one α-synuclein B cell epitope, or a combination of at least one amyloid-β (Aβ) B cell epitope and at least one Tau B cell epitopes and at least one α-synuclein B cell epitope, and at least one foreign T helper cell (Th) epitope.

In certain embodiments, compositions herein are used to generate an immune response in a subject in need thereof, comprising administering the immunogen to the subject. The subject in need may be at risk of developing or has been diagnosed with Alzheimer's disease or one or more conditions associated with abnormal amyloid deposits, Tau deposits, and α-syn deposits. The compositions can be used to prevent, treat or ameliorate a condition associated with deposits of amyloid, tau, and/or α-syn, comprising administering to a subject in need thereof an effective amount of the immunogen. In certain embodiments, the present technology is directed to

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show four graphs showing the immunogenicity in mice of trivalent influenza vaccine (TIV) formulated with the TLR9 agonist PAMP CpG2006, highlighting the synergistic effect when inulin particles are added to the CpG-containing TIV vaccine formulation. Female Balb/c mice at 6-8 weeks of age (n=5-8 per group) were immunized intramuscularly twice 14 days apart, with 50 ul of a commercial human TIV at 100 ng HA per dose, combined with either 2, 7, 20 or 60 μg of CpG2006 alone or mixed with 1 mg PDmix(1:5). FIG. 1A shows serum anti-influenza total IgG levels, FIG. 1B shows serum anti-influenza IgM levels, FIG. 1C shows serum anti-influenza IgG1 levels and FIG. 1D shows serum anti-influenza IgG2a levels 42 days after the second immunization as measured by ELISA. Shown are group mean OD+SD.

FIGS. 2A-2F show six graphs demonstrating the synergistic effects of a combination of the TLR9 agonist PAMP (CpG1668) and inulin particles (PDmix1:36) on the immune response of neonatal mice to TIV vaccine. Neonatal BALB/c mice (n=5-7/group) were immunized i.m. with TIV (100 ng total HA protein) at 14 days and 23 days of age. Sera were collected 14 days after the last injection for measurement of antibodies by ELISA. Groups received either TIV alone or formulated with PDmix1:36 (1 mg), CpG1668 (20 ug), or PDmix (1 mg)+CpG1668 (20 ug). FIG. 2A shows the group receiving TIV+PDmix+CpG (final column in each figure) had significantly higher anti-influenza total IgG, FIG. 2B shows higher IgM, FIG. 2C shows lower IgG1, FIG. 2D shows higher IgG2a, FIG. 2E shows higher anti-influenza CD4+ T cell and FIG. 2F shows higher CD8+ T-cell memory responses.

FIGS. 3A-3D show four graphs showing the anti-influenza IgM, IgG2a, IgG1, and total IgG responses as measured by ELISA in sera from 200-300 day old female Balb/c mice (n=10/group) immunized intramuscularly twice 14 days apart with a trivalent inactivated influenza vaccine. FIG. 3A shows serum anti-influenza total IgG levels, FIG. 3B shows serum anti-influenza IgM levels, FIG. 3C shows serum anti-influenza IgG1 levels and FIG. 3D shows serum anti-influenza IgG2a levels 42 days after the second immunization as measured by ELISA. Shown are group mean OD+SD. The group co-administered inulin particles (PDmix) plus a TLR9 agonist PAMP (CpG2006) achieved the highest anti-influenza antibody titers.

FIGS. 4A-4D show four graphs showing the immunogenicity in mice of trivalent influenza vaccine (TIV) formulated with an inulin particle formulation PDmix alone or combined with a range of TLR9 agonist PAMPs. FIG. 4A shows serum anti-influenza total IgG levels, FIG. 4B shows serum anti-influenza IgM levels, FIG. 4C shows serum anti-influenza IgG1 levels and FIG. 4D shows serum anti-influenza IgG2a levels, 28 days after the second immunization as measured by ELISA. Shown are group mean OD+SD. The co-administration of TIV with PDmix and either CpG1668, CpG2006 or CpG2395 all showed synergy over the individual components in increasing anti-influenza total IgG, IgG2a and IgM titers. CpG2216 and CpG2237 had no effect on the antibody response.

FIGS. 5A-5D show four graphs showing the immunogenicity in mice of rabies vaccine (MIRV) formulated with either of two inulin particle formulations (dIN or PDmix) alone or combined with a TLR9 agonist CpG1668. FIG. 5A shows serum anti-rabies total IgG levels, FIG. 5B shows serum anti-rabies IgM levels, FIG. 5C shows serum anti-rabies IgG1 levels and FIG. 5D shows serum anti-rabies IgG2a levels 14 days after the second immunization as measured by ELISA. Shown are group mean OD+SD. The combination of either dIN or PDmix with CpG1668 plus MIRV provided the highest anti-rabies total IgG, IgG1, IgG2a and IgM.

FIGS. 6A-6D show four graphs showing the immunogenicity in mice of trivalent influenza vaccine (TIV) formulated with an inulin particle formulation PDmix alone or combined with a range of TLR2 agonist PAMPs (zymosan, LTA, Lipomannan and PamCSK4) as compared to the TLR9 agonist PAMP CpG2006. FIG. 6A shows serum anti-influenza total IgG levels, FIG. 6B shows serum anti-influenza total IgM levels, FIG. 6C shows serum anti-influenza IgG1 levels and FIG. 6D shows serum anti-influenza IgG2a levels 14 days after the second immunization as measured by ELISA. Shown are group mean OD+SD.

FIGS. 7A-7C show three graphs showing the favorable immune enhancing effect of combinations of inulin particles with various PAMPs on immunogenicity in mice of TIV vaccine. FIG. 7A shows serum anti-influenza total IgG levels, FIG. 7B shows serum anti-influenza IgG1 levels and FIG. 7C shows serum anti-influenza IgG2a levels 42 days after the second immunization as measured by ELISA. Shown are group mean OD+SD.

FIGS. 8A-8F show six graphs showing the favorable immune enhancing and antigen-sparing effect of combinations of inulin particles (dIN) with a TLR9 agonist PAMP, CpG2006 on immunogenicity in mice of a recombinant pandemic influenza vaccine, rH5. Balb/c mice at 6-8 weeks of age (n=5-8/group) were immunized intramuscularly twice 21 days apart, with 50 μl of a vaccine formulation containing between 3 ng and 3 μg of influenza recombinant H5 (rH5) serotype hemagglutinin protein (rH5) (Protein Sciences Corp, Meriden, USA) plus either dIN 1 mg or dIN 1 mg mixed with CpG2006 5 μg. FIG. 8A shows serum anti-H5 total IgG, FIG. 8B shows anti-H5 IgM, FIG. 8C shows anti-H5 IgG1, FIG. 8D shows anti-H5 IgG2a, FIG. 8E shows anti-H5 IgG2b, and FIG. 8F shows anti-H5 IgG3 14 days after the second immunization as measured by ELISA. Shown are group mean OD+SD.

FIGS. 9A-9B show 2 graphs showing the favorable immune enhancing effect of combinations of inulin particles (dIN) with a TLR9 agonist PAMP, CpG2006 together with H1N1 PR8 vaccine on survival of mice after challenge with lethal PR8 virus dose. FIG. 9A shows mice receiving combinations of inulin particles (dIN) with a TLR9 agonist PAMP, CpG2006 together with H1N1 PR8 vaccine had complete protection with no weight loss or clinical disease, whereas PR8+dIN without CpG was only partially protective. FIG. 9B shows again in a separate study that mice receiving combinations of inulin particles (dIN) with a TLR9 agonist PAMP, CpG2006 together with H1N1 PR8 vaccine were protected against death, whereas PR8+CpG gave no protection.

FIGS. 10A-10D show four graphs that show the hemagglutination inhibition titers (HI) (FIGS. 10A and 10B) and microneutralization (MN) (FIGS. 10C and 10D) titers in immunized ferrets measured at the time of the booster dose (21 days prior to challenge) and 14 days after the booster dose (7 days prior to challenge). Ferrets vaccinated with two doses of H5N1 with Ad2 had the highest neutralizing antibody titers, consistent with enhanced immune response when H5N1 antigen was combined with a formulation of inulin particles plus a TLR9 agonist.

FIG. 11 shows a graph showing enhanced (100%) survival post lethal H5N1 challenge in ferrets that received Ad1- or Ad2-adjuvanted H5N1 vaccine, including the group that received just one immunization with 22.5 μg H5N1 vaccine+Ad2. Each of the 10 groups is denoted by survival percent: vaccine dose (or saline)+adjuvant identify (or saline). The survival of the five adjuvanted-vaccine groups were significantly greater than for the two unadjuvanted vaccine groups (Log-Rank test, p=0.05) and from the three unvaccinated control groups (Log-Rank test, p<0.001).

FIGS. 12A-12G show seven graphs that show the group mean weight change in immunized ferrets post challenge with H5N1 virus. Ferrets vaccinated with two doses of H5N1 with Ad2 did not lose any weight, consistent with enhanced protection when the H5N1 antigen was combined with a formulation of inulin particles plus a TLR9 agonist.

FIGS. 13A-13G show seven graphs that show the group mean temperature change in immunized ferrets post lethal challenge with H5N1 virus. While four ferrets in the Ad1 (inulin article alone)-adjuvanted vaccine groups demonstrated fever, no ferrets in the Ad2 (inulin particle+CpG)-adjuvanted group experienced fever, consistent with enhanced protection when the H5N1 antigen was combined with a formulation of inulin particles plus a TLR9 agonist.

FIGS. 14A-14C show three graphs that show gIN, dIN or eIN all had a synergistic enhancing effect with the CpG in the induction of anti-HBsAg IgG1, IgG2a and IgM consistent with the synergistic effect on PAMP innate immune activators being a shared property of different polymorphic forms of inulin particles. Adult Balb/c mice were immunized intramuscularly twice 21 days apart, with HBsAg together with either gIN, dIN or eIN inulin particles alone or together with the TLR9 PAMP, CpG2006. FIG. 14A shows serum anti-HBsAg IgG1, FIG. 14B shows serum anti-HBsAg IgG2a levels and FIG. 14C shows serum anti-HBsAg IgGM levels after the second immunization as measured by ELISA. Shown are group mean OD+SD.

FIG. 15 illustrates the mechanism of action for an epitope vaccine. Adjuvant and delivery systems support the efficient delivery of the vaccine to the immune system. Antigen-presenting cells uptake delivered vaccine and present the antigen to T helper cells specific to Th epitopes incorporated into the vaccine. B cells recognize the active component of the vaccine (B cell epitope) by B cell receptors (first signal for activation) and simultaneously present the Th epitope of the vaccine to the same T helper cells activated by APC creating B cell/T cell synapse. Thus, B cells specific to Aβ₁₁ bind the antigen via a B cell receptor (first signal) and get help from activated Th cells (second signal). B cells that are activated in this way begin to produce specific antibodies.

FIGS. 16A-16B show design of exemplary vaccines. FIG. 16A shows a schematic representation of constructs encoding various types of epitope vaccines. Parental construct (p3Aβ₁₁-PADRE) was modified to express the same three copies of active component, Aβ₁₁ B cell epitopes (one epitope with free N-terminal aspartic acid) fused with nine (AV-1955) or twelve (AV-1959) different, promiscuous foreign Th cell epitopes each separated by a neutral spacer with few amino acids (for example, a glycine-serine spacer). Using such constructs one may generate appropriate recombinant proteins. FIG. 16B shows the origin and sequence of various CD4+ T cell epitopes forming the Th epitope strings for AV-1955 and AV-1959 vaccines (designated collectively as the MultiTEP platform) (SEQ ID NO: 45).

FIGS. 17A-17B are photographs of a Western blot. Correct cleavage of signal sequence and generation of free N-terminus aspartic acid in a first copy of Aβ₁₁ in AV-1955 was analyzed in conditioned media (CM) of CHO cells transfected with p3Aβ₁₁-PADRE-Thep (Lane 1) and AV-1955 (Lane 2) by IP/WB. Both proteins were immunoprecipitated with 6E10 monoclonal antibodies (Mab) and blots were stained with 6E10 (FIG. 17A) or rabbit antibody specific to the N-terminus of Aβ peptide (FIG. 17B).

FIGS. 18A-18B show results of immunization of mice by gene gun with MultiTEP based AD epitope vaccines AV-1959, AV-1955 and p3Aβᵢᵢ-PADRE. FIG. 18A shows cellular response measured as IFNγ SFC per 10⁶ splenocytes; FIG. 18B shows humoral immune responses measured by concentration of anti-Aβ antibodies in μg/mL.

FIGS. 19A-19C present graphs showing results of immunization with MultiTEP based AD epitope vaccine AV-1959. FIG. 19A shows that cellular immune responses are specific to Th epitopes incorporated into the vaccine but not to Aβ₄₀, and FIGS. 19B and 19C show anti-Aβ antibodies in mice, rabbits and monkeys.

FIGS. 20A-20C present results of Rhesus macaques vaccinated with MultiTEP based AD epitope vaccine showing therapeutic potency. Anti-Aβ antibody purified from sera of vaccinated monkeys but not irrelevant monkey IgG binds to cortical plaques in AD brain (FIG. 20A) and to immobilized Aβ₄₂ monomeric, oligomeric, or fibrillar forms as measured using the Biacore (FIG. 20B). Anti-Aβ antibody inhibits Aβ₄₂ fibrils- and oligomer-mediated neurotoxicity (FIG. 20C).

FIGS. 21A-21B show data obtained from APP/Tg mice vaccinated with MultiTEP based AD epitope vaccine. FIG. 21A shows induced anti-Aβ₁₁ antibody significantly reduced diffuse and dense-core Aβ-plaques detected by staining with 6E10 and dense-core plaques detected by staining with ThS. FIG. 21B shows soluble and insoluble Aβ detected by biochemical methods.

FIG. 22 shows T cell responses after re-stimulation. Inbred mice of H2b haplotype were vaccinated with MultiTEP based AV-1959 vaccine and restimulated in vitro with different epitopes from the vaccine.

FIGS. 23A-23B show responses of individual, out-bred macaques to different Th cell epitopes after immunization. FIG. 23A shows mapping of Th cell epitopes in non-inbred macaques with high MHC class II polymorphism. FIG. 23B presents the analyses of prevalence of Th epitopes within the NHP population.

FIGS. 24A-24C present a schematic representation of experimental design (FIG. 24A) demonstrating the immunological potential of pre-existing Th cells and results. FIG. 24B shows cellular response and FIG. 24C shows humoral response after immunization with multi-TEP protein in QuilA or QuilA alone and boosted with AV-1959.

FIG. 25A shows overlapping peptides of α-syn used for mapping immunodominant B cell epitopes. FIG. 25B shows a schematic representation of epitope vaccine based on α-syn B cell epitope fused to MultiTEP platform.

FIGS. 26A-26B present data of immune responses in mice vaccinated with an α-Synuclein epitope-based vaccine. FIG. 26A shows antibody concentration following immunization with α-Syn₃₆₋₆₉-MultiTEP or irrelevant peptide. FIG. 26B shows cellular response to MultiTEP and to α-synuclein.

FIGS. 27A-27C show antibody responses to different portions of α-Synuclein. Mice were immunized with epitope vaccine based on K10AKEG14 calpain cleavage site of α-Synuclein α-Syn₁₀₋₁₄-MultiTEP (FIG. 27A). Antibody binding to α-Syn₁₀₋₁₈ peptide (FIG. 27B) and to full length α-Synuclein protein (FIG. 27C).

FIG. 28 shows results of mapping of immunodominant B cell epitopes in tau protein. Mice were immunized with 4R/2N Tau protein. Binding of generated antibodies to 50-mer peptides comprising tau protein was analyzed by ELISA.

FIGS. 29A-29C present data of immunization of B6SJL mice with Tau₂₋₁₈ fused with a foreign Th cell epitope. FIG. 29A shows titers of antibody specific to tau₂₋₁₈ peptide were determined in serially diluted individual sera. Lines indicate the average of mice. FIG. 29B shows binding of anti-Tau₂₋₁₈ antibodies to wild/type (4R/0N), mutated P301L and deleted (Δ19-29) tau proteins of 4R/0N isoform (dilution of sera 1:600. Lines indicate the average of OD450). FIG. 29C shows detection of IFN-γ producing cells in the cultures of immune splenocytes activated with P30 peptide and tau₂₋₁₈. The number of IFNγ producing splenocytes was analyzed by ELISPOT assay after ex vivo re-stimulation of cells with 10 μg/mL tau₂₋₁₈ and P30 peptides. Error bars indicate average±s.d. (P≦0.001).

FIG. 30 presents photographs of immunostaining of brain sections of patients with Alzheimer's Disease (AD) case and normal non-AD case patients. Antibodies include anti-tau₂₋₁₈ sera from mice immunized with tau₂₋₁₈-P30 (left panels), known anti-tau antibodies (middle panels) and control antisera from mice immunized with an irrelevant antigen (BORIS) (right panels).

FIGS. 31A-31B present results of antibody blocking brain lysate induction of aggregation of intracellular tau repeat domain (RD). FIG. 31A shows brain lysate was either untreated or treated with anti-tau₂₋₁₈ antibody and added to HEK293 cells co-transfected with RD(ΔK)-CFP/YFP prior to FRET analysis. Increased FRET signal was detected in wells with untreated brain lysate. Treatment of lysate with anti-tau₂₋₁₈ antibody decreased FRET signal to the baseline level due to blocking the full-length tau in brain lysate and inhibition of induction of RD aggregation. FIG. 31B shows confocal microscope images of exemplary binding of anti-tau₂₋₁₈ antibody/brain lysate complexes to HEK293 cells transfected with RD-YFP. Secondary anti-mouse immunoglobulin conjugated with Alexa546 was used.

FIGS. 32A-32B present data of anti-tau₂₋₁₈ antibody blocking the trans-cellular propagation of tau RD aggregates. FIG. 32A shows HEK293 cells transfected with RD(LM)-HA were co-cultured for 48 h with an equivalent number of HEK293 cells co-transfected with RD(ΔK)-CFP/YFP prior to FRET analysis. Increased FRET signal was detected in co-cultured cells. Addition of serial dilutions of purified mouse anti-tau₂₋₁₈ or rat anti-tau₃₈₂₋₄₁₈ antibody decreased FRET signal due to inhibition of trans-cellular propagation of aggregated RD. FIG. 32B shows binding of anti-tau₂₋₁₈ antibodies HEK293 cells transfected with RD(ΔK)-YFP or were mock-transfected (NT) was analyzed by confocal microscope. Anti-tau₂₋₁₈ antibody was added to the culture medium for 48 h. Cells were fixed, permeabilized, and stained with an anti-mouse secondary antibody labeled with Alexa 546 and analyzed by confocal microscopy. Anti-tau₂₋₁₈/RDΔ(K)-YFP complexes were identified when RDΔ(K)-YFP is expressed but not in its absence (NT).

FIG. 33 shows schematics of exemplary multivalent DNA epitope vaccines based on MultiTEP platform. AV-1953 is bivalent epitope composed of 3 copies of Aβ₁₁ and 3 copies of tau₂₋₁₈ epitopes fused to MultiTEP platform. AV-1950 and AV-1978 are trivalent vaccines containing α-syn epitopes KAKEG and α-syn₃₆₋₆₉, respectively, in addition to Aβ and tau.

FIGS. 34A-34C show data from immunization of wildtype mice with bivalent and trivalent DNA epitope vaccines. FIG. 34A shows anti-Aβ₄₂ and anti-Tau antibody responses generated by bivalent AV-1953 vaccine. FIG. 34B shows anti-Aβ₄₂, anti-Tau and anti-α-syn antibody responses generated by AV-1978 trivalent vaccine. Ab responses were measured in sera of individual mice by ELISA and lines represent the average value of Ab. Concentration of Ab specific to α-syn and Aβ₄₂ was calculated using a calibration curve generated with mouse anti-α-syn and 6E10 anti-Aβ₄₂ antibodies, respectively. Endpoint titers of anti-Tau antibodies were calculated as the reciprocal of the highest sera dilution that gave a reading twice above the cutoff. The cutoff was determined as the titer of pre-immune sera at the same dilution. FIG. 34C shows trivalent vaccine AV-1978 activated Th cells specific to epitopes of MultiTEP platform but not to B cell epitopes. IFNγ producing cells in the cultures of immune splenocytes were detected by ELISPOT after in vitro re-stimulation of cells with indicated peptides/proteins. Error bars indicate average±s.d. (n=6).

FIGS. 35A-35B show humoral immune responses in mice vaccinated with AV-1959R protein formulated with cGMP grade adjuvants (Advax^{CpG}, Advax™, Montanide-ISA51, Montanide-ISA720, MPLA, Alhydrogel®) and control adjuvant, Quil-A. As shown in FIG. 35A, concentrations of anti-Aβ antibodies were measured by ELISA in sera collected after the 3^{rd} immunization. Lines represent mean values. As shown in FIG. 35B, isotypes of generated anti-Aβ antibodies had been determined by ELISA and IgG1/IgG2a^{b} ratio was calculated. Bars represent average±SD (n=6-8/per group). Statistical significance was calculated against group of mice immunized with AV-1959R formulated in Advax^{CpG} using ANOVA test (**P<0.01***, P<0.001 and ****P<0.0001).

FIGS. 36A-36C show cellular immune responses in mice vaccinated with AV-1959R protein formulated with cGMP grade adjuvants (Advax^{CpG}, Advax™, Montanide-ISA51, Montanide-ISA720, MPLA, Alhydrogel®) and control adjuvant, Quil-A. As shown in FIGS. 36A and 36B, numbers of IFN-γ (A) and IL-4 (B) producing T cells were calculated by ELISpot in splenocyte cultures obtained from experimental and control animals. (C) IL-4/IFN-γ ratios were calculated based on data presented in FIGS. 36A and 36B. Bars represent average±SD (n=6-8/per group). Statistical significances were calculated against group of mice immunized with AV-1959R formulated in Advax^{CpG} using ANOVA test (*P<0.05, **P<0.01, ***P<0.001 and ****P<0.0001).

FIGS. 37A-37C show cellular immune responses in mice immunized with epitope vaccines targeting Aβ (AV-1959R), tau (AV-1980R), and Aβ/tau together in one construct (dual epitope vaccine): AV1953R or a mixture of AV1959R and AV-1980R) (AV1959R+AV1980R). Numbers of IFN-γ (FIG. 37A) and IL-4 (FIG. 37B) producing T cells were calculated by ELISpot in splenocyte cultures obtained from experimental and control animals. As shown in FIG. 37C, proliferation of cells was detected by [3H]-thymidine incorporation assay in the same splenocyte cultures and expressed as stimulation index. Cellular immune responses in control group were at the background level (INF-γ⁺ and IL-4⁺ SFCs were <15, and stimulation index was <1.6). Bars represent average±SD (n=8 per group).

FIGS. 38A-38B show humoral immune responses in mice vaccinated with AV-1959R, AV-1980R, AV-1953R and AV-1959R+AV-1980R formulated with Advax^{CpG} adjuvant. Concentrations of anti-Aβ (FIG. 38A) and anti-tau (FIG. 38B) antibodies were measured by ELISA in sera collected after the 3^{rd} immunization and calculated using calibration curves generated with 6E10 and 1C9 monoclonal antibodies, respectively. Lines represent mean values for n=8/per group (*P<0.05, **P<0.01, ANOVA test).

FIGS. 39A-39B show numbers of B cells producing anti-Aβ and anti-Tau antibodies in mice vaccinated with AV-1959R, AV-1980R, AV-1953R and AV-1959R+AV-1980R formulated with Advax^{CpG} adjuvant. Detection of anti-Aβ (FIG. 39A) and anti-tau (FIG. 39B) antibody-secreting cells (ASC), visualized as spots, was done in splenocyte cultures obtained from experimental and control mice using ELISpot assay. Bars represent average±SD (n=8/per group, *P<0.05, ANOVA test).

FIGS. 40A-40F show 3D structural models of AV-1980R, AV-1959R and AV-1953R synthetic proteins. The surface filled representations of the AV-1980R (FIG. 40A), AV-1959R (FIG. 40B) and AV1953R (FIG. 40C) are presented in the upper panel. Tau and Aβ epitopes on the MultiTEP protein are highlighted in pink and red, respectively. The GS linker is highlighted in dark grey. In the lower panel, critical residues on the AV-1980R epitope (PRQEF) are highlighted in blue (FIG. 40D) and the critical residues on the AV-1959R epitope (EFRH) are highlighted in cyan (FIG. 40E). In AV-1953R critical residues on each epitope follows AV-1980R and AV-1959R color cording (FIG. 40F).

FIGS. 41A-41C show that immune sera isolated from mice vaccinated with AV-1959R, AV-1980R, AV-1953R and AV-1959R+AV-1980R formulated with Advax^{CpG} adjuvant bound to different forms of Aβ and tau in the brains from AD cases. Western blots of soluble (FIG. 41A) and insoluble fractions (FIG. 41B) of brain homogenates containing 50 μg total protein from four AD cases were stained with immune sera normalized to 1 μg/ml for anti-Aβ and 0.4 μg/ml for anti-tau antibodies based on ELISA data. (FIG. 41C) Immune sera were screened for the ability to bind to human Aβ plaques or/and tau tangles using 40 μm brain sections of formalin-fixed cortical tissue from the same AD cases. The original magnification is 60× and the scale bar is 20 μm.

FIGS. 42A-42B show humoral and cellular immune responses in mice vaccinated twice with AV-1959R and boosted (single boost) with AV-1980R formulated with Advax^{CpG} adjuvant. (FIG. 42A) Numbers of IFN-γ producing cells were detected by ELISpot in splenocyte cultures. Bars represent average±SD for n=4/per group. (FIG. 42B) Concentrations of anti-tau antibodies were measured by ELISA. Lines represent mean values for n=10/per group (*P<0.05, **P<0.01, t-test).

FIG. 43 shows humoral immune responses in PS19 mice vaccinated with AV-1980R formulated with Advax^{CpG} adjuvant. Concentrations of anti-tau antibodies were measured by ELISA in sera collected after the 2^{nd}, 3^{rd} and 4^{th} immunizations and calculated using calibration curves generated with 1C9 anti-tau₂₋₁₈ monoclonal antibodies.

FIGS. 44A-44B show humoral immune responses in T5×APP/Tau double transgenic mice vaccinated with AV-1959R, AV-1980R and AV1959R+AV1980R vaccines formulated with Advax^{CpG} adjuvant. Concentrations of anti-Aβ (FIG. 44A) and anti-tau (FIG. 44B) antibodies were measured by ELISA in sera collected after the 2^{nd} and 3^{rd} immunizations and calculated using calibration curves generated with anti-Aβ6E10 and anti-tau₂₋₁₈ 1C9 monoclonal antibodies.

FIG. 45 shows anti-Tau antibody responses in rTg4510 transgenic mice immunized with AV-1980R formulated in Advax^{CpG} adjuvant after 2^{nd}, 3^{rd} and 4^{th} immunizations. Concentrations of anti-Tau antibodies were calculated using calibration curves generated with 1C9 anti-tau₂₋₁₈ monoclonal antibodies.

FIG. 46 shows cellular immune anti-MultiTEP responses in rTg4510 transgenic mice immunized with AV-1980R formulated in Advax^{CpG} adjuvant. Numbers of IFN-γ producing T cells were calculated by ELISpot in splenocyte cultures obtained from experimental and control animals and re-stimulated in vitro with cocktail of Th peptides incorporated into MultiTEP platform or with Tau₂₋₁₈ peptide. Bars represent average±SD (n=6).

FIG. 47 shows humoral immune responses in young and old hα-Syn Tg mice vaccinated with AV-1950R epitope vaccine targeting three different epitopes of hα-Syn. Young mice were immunized at the age of 3 mo and titers of anti-ha-Syn antibodies were determined in sera of vaccinated mice after 3^{rd} immunization. Old mice were immunized at the age of 12-14 mo and titers of anti-ha-Syn antibodies were determined in sera of vaccinated mice after 2^{nd} immunization. Endpoint titers of antibodies specific to recombinant hα-Syn are calculated as the reciprocal of the highest sera dilution that gave a reading twice above the background levels of pre-immune sera at the same dilution (cutoff).

FIG. 48 shows antibody concentrations in Tg2576 mice vaccinated with AV1959R formulated in Advax^{CpG} adjuvant and LU AF20513 formulated in Alhydragel. Mean concentrations of antibodies are shown.

FIG. 49 shows antibody titers in PS19, rTg4510 and T5× mice vaccinated with AV1980R formulated in Advax^{CpG} adjuvant. Table compares antibody titers in PS19 mice after immunization with AV1980R formulated in Advax^{CpG} adjuvant and liposome-based vaccine ACI-35 containing MPLA adjuvant

FIG. 50 shows a schematic representation of vaccines targeting different B cell epitopes of hα-Syn: aa85-99 (PV-1947), aa109-126 (PV-1948), aa126-140 (PV-1949) and all three epitopes together with reverse order (aa126-140+aa109-126+aa85-99; PV-1950).

### DETAILED DESCRIPTION

The listing or discussion of any apparently prior-published document in this specification should not necessarily be taken as an acknowledgement that the document is part of the state of the art or is common general knowledge.

The practice of the present technology will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984), Current Protocols in Immunology ISBN 9780471522768 (Publisher: John Wiley and Sons Inc.), Vaccine Adjuvants and Delivery Systems (Manmohan Singh ed. 2007), Methods in Molecular Biology, ISBN 9781607615842 (Publisher: Springer), History of Vaccine Development 2011, ISBN:1441913386 (Publisher: Springer)

Unless defined otherwise, all technical and scientific terms used herein have the same meaning as commonly understood by one of ordinary skill in the art to which this technology belongs. Although any methods and materials similar or equivalent to those described herein can be used in the practice or testing of the technology, the preferred methods and materials are now described.

As known to those experienced in the art, innate immune activation can be used to enhance the type or magnitude of an adaptive immune memory response. Enhancement or modulation of the adaptive immune response is advantageous during vaccination or during allergen desensitization, as it can provide a means to magnify or extend the duration of the immune memory response against a particular pathogen, or alter the type of immune response to a more beneficial response. For example, for some pathogens, it may be advantageous to induce a strong antibody (Th2) response to the immunizing antigen, while for other pathogens, it may be advantageous to induce a strong Th1 response or a strong Th17 response. On the other hand, for antigens such as allergens it may be advantageous to suppress the existing IgE response and instead induce a Th1 response to the allergen. It has been discovered according to the current technology that the combination of inulin particles with an innate immune activator enables a variety of unique patterns of immune response to be obtained that can, for example, be used to modulate the adaptive immune memory response to a co-administered antigen to a favored type or direction.

A first aspect of the present technology provides a composition comprising inulin particles for use in the reduction or inhibition of inflammation, and/or for treating or preventing inflammatory disease, in a subject.

A second aspect of the present technology provides an immunological and/or pharmaceutically acceptable composition comprising (a) an anti-inflammatory component, such as inulin particles and/or one or more other anti-inflammatory inhibitors of IL-1; together with (b) a substance comprising one or more species of an innate immune activator such as a pathogen-associated molecular pattern (PAMP). Without wishing to be bound by theory, a favorable immune interaction occurs because each of the two components of the immunological composition regulate transcription of an independent set of immune genes, such that the pattern of immune genes expressed in response to the combined immunological composition is unique to the combination and different to the patterns of gene expression induced by the individual components.

A third aspect of the present technology provides a kit of parts comprising: (a) a first container that contains a composition comprising an anti-inflammatory component, such particles of inulin and/or one or more other anti-inflammatory inhibitors of IL-1 (as discussed above in respect of the second aspect of the present technology); and (b) a second container that contains a substance comprising a pathogen-associated molecular pattern (PAMP).

Thus, component (a) of the composition of the second aspect of the present technology, or the kit of the third aspect of the technology, comprises anti-inflammatory component, such as an anti-inflammatory inhibitor of IL-1 or an anti-inflammatory inhibitor of NFκB.

In certain embodiments, the anti-inflammatory component comprises inulin particles. The term “inulin particle” as used herein refers not only to particles made from β-D-(2-1)polyfructofuranosyl-α-D-glucose (also known as inulin) but also to derivatives thereof such as β-D-(2-1) polyfructose which may be obtained by enzymatic removal of the end glucose from inulin, for example using an invertase or inulase enzyme capable of removing the end glucose. The term inulin particle also refers to any natural or synthetic particle that is constituted by, contains or is coated by inulin, or a derivative or mimetic thereof. Suitable inulin derivatives included within the scope of this term are derivatives of inulin in which the free hydroxyl groups have been acetylated, methylated, etherified or esterified, for example by chemical substitution with alkyl, aryl or acyl groups, by known methods. The stable inulin particle may be solid or hollow and may be wholly comprised of inulin molecules or may alternatively have a non-sugar core, skeleton or shell comprising, for example, carbohydrate compounds, metal compounds, proteins or lipids but which at its surface expresses inulin molecules either covalently or non-covalently bonded to the components comprising the core. Preferably, the inulin particle will be selected from the group of gIN, dIN and eIN, or modifications thereof. Most preferably, the inulin particle will be dIN. Preferably, the inulin particle will have a diameter in the size range of 20 nM to 20 μM. More preferably, the inulin particle will have a diameter in the size range of 0.1 to 5 μM. Most preferably the inulin particle will have a diameter in the size range of 0.5 to 5 μM.

In certain embodiments, inulin particles as used in the present technology are stable inulin particles. The term “stable” as used herein refers to an inulin particle that is totally insoluble or predominantly insoluble or partially insoluble at the body temperature of the subject to whom it is to be administered. In this context, stability may optionally include the meaning that the inulin particles are insoluble when incubated at a temperature of up to 25° C. or up to 30° C., 37° C., 40° C., 42° C., 45° C., 48° C., 50° C., 52° C., 55° C., 58° C., or 60° C. when present at a concentration of no greater than 0.5 mg/mL or 1 mg/mL or 2 mg/mL in distilled water or saline or phosphate buffered saline, for at least 10, 20, 30, 40, 50, or 60 minutes. The amount of insoluble inulin can be measured by changes in the optical density of the inulin suspension at 300 nm, 400 nm, 500 nm, 600 nm, 700 nm wavelength (OD₇₀₀) using a spectrophotometer and, in this context, an inulin particle can be said to be stable if it remains insoluble at the defined condition as indicated by the OD₇₀₀ not falling below a value that is 50%, 60%, 70%, 80%, 90%, 95%, 98%, 99%, 99.5%, 99.9% or substantially 100% of the OD₇₀₀ of the particle preparation in the same solvent and at the same concentration prior to incubation at the defined temperature (preferably when measured at a temperature that is 10° C. or more below the incubation temperature)

Other anti-inflammatory components, which may be used in component (a) of the composition of the second aspect, or the kit of the this aspect of the technology, instead of or as well as, inulin particles, may include—

(i) inhibitors of the IL-1 pathway genes or proteins, particularly those that are functionally-equivalent to inulin particles, in the sense of possessing an essentially equivalent anti-inflammatory property, activity and/or specificity and/or possessing an essentially equivalent immunomodulatory or adjuvant property,

(ii) one or more of IL1 receptor antagonists, IL1RA, Anakinra, Rilonacept, IL-1R/IL1RacP/Fc-fusion protein, Canakinumab, a human IL-1β antibody, IL1 receptor blockers, IL-1RII, indomethacin, non-steroidal anti-inflammatory drugs (NSAID), glucocorticoids, caspase inhibitors including caspase 1 inhibitors, inflammasome inhibitors including NALP3 antagonists, curcumin, resveratrol, chloroquine, P2X7 receptor inhibitors, ST2 receptor inhibitors, and/or ATP antagonists;

(iii) agents that up-regulate or activate the anti-inflammatory protein peroxisome proliferator-activated receptor gamma (PPAR-γ) or upregulate genes or proteins in the PPAR-γ pathway, particularly in monocytes and dendritic cells (PPAR-γ pathway genes are also upregulated by inulin particles). PPAR-γ upregulation has been previously shown to inhibit inflammatory responses including suppressing LPS-induced IL-1 and TNFα and conversely IL1 and TNFα PPAR-γ. Suitable agents may include one or more of rosiglitazone, pioglitazone, prostaglandin J2, curcumin, resveratrol, thiazolidenediones, Berberine, perfluorononanoic acid, RS5444, free fatty acids, vitamin D, and/or eicosanoids.

(iv) anti-inflammatory agents such as aspirin, ibuprofen, and naproxen, salicylic acid, submandibular gland peptide-T, phenylalanine-glutamine-glycine (FEG), ginger, turmeric, sesquiterpene lactone, Omega-3 fatty acids, prostaglandin-E, prostaglandin-E3, Curcumin, Mesalazine, Selective glucocorticoid receptor agonist, Lisofylline, Mofezolac, Oleocanthal, Ibuproxam, Cyclopentenone, prostaglandin, Cannabidiol, BMS-345541, BMS-470,539, Amlexanox, Amixetrine, Allicin, Actarit, Butylpyrazolidines, for example, Phenylbutazone; Mofebutazone; Oxyphenbutazone; Clofezone; Kebuzone; Suxibuzone; Acetic acid derivatives and related substances, such as Indometacin; Sulindac; Tolmetin; Zomepirac; Diclofenac; Alclofenac; Bumadizone; Etodolac; Lonazolac; Fentiazac; Acemetacin; Difenpiramide; Oxametacin; Proglumetacin; Ketorolac; Aceclofenac; Bufexamac; Indometacin, Diclofenac, Oxicams, such as Piroxicam; Tenoxicam; Droxicam; Lornoxicam; Meloxicam; Propionic acid derivatives, such as Ibuprofen; Naproxen; Ketoprofen; Fenoprofen; Fenbufen; Benoxaprofen; Suprofen; Pirprofen; Flurbiprofen; Indoprofen; Tiaprofenic acid; Oxaprozin; Ibuproxam; Dexibuprofen; Flunoxaprofen; Alminoprofen; Dexketoprofen; Naproxcinod; Naproxen and esomeprazole; Naproxen and misoprostol; Vedaprofen; Carprofen; Tepoxalin. Fenamates, such as Mefenamic acid; Tolfenamic acid; Flufenamic acid; Meclofenamic acid; Flunixin, Coxibs, such as Celecoxib; Rofecoxib; Valdecoxib; Parecoxib; Etoricoxib; Lumiracoxib; Firocoxib; Robenacoxib; Mavacoxib; Cimicoxib, Other anti-inflammatory and antirheumatic agents, such as Nabumetone; Niflumic acid; Azapropazone; Glucosamine; Benzydamine; Glucosaminoglycan polysulfate; Proquazone; Orgotein; Nimesulide; Feprazone, Diacerein; Morniflumate; Tenidap; Oxaceprol, Chondroitin sulfate; Avocado and soybean oil, unsaponifiables, Niflumic acid, Feprazone, combinations; Pentosan polysulfate; Aminopropionitrile; Anti-inflammatory/antirheumatic agents in combination with corticosteroids, such as Phenylbutazone and corticosteroids; Dipyrocetyl and corticosteroids; Acetylsalicylic acid and corticosteroids; Specific antirheumatic agents including Quinolines, such as Oxycinchophen, Gold preparations, such as Sodium aurothiomalate; Sodium aurothiosulfate; Auranofin; Aurothioglucose; Aurotioprol, and/or Penicillamine and similar agents, such as Bucillamine.

As a general rule, the inulin particle (or other equivalent anti-inflammatory component) can be used in an amount of 0.001 mg and 100 mg per kilogram body weight of the subject to be immunized. For example, the inulin particle (or other equivalent anti-inflammatory component) of a composition of the present technology may be present at a concentration in the range of 0.1 mg to 100.0 mg per kilogram body weight. In another example, the inulin particle (or other equivalent anti-inflammatory component) of the composition may be administered to an adult human subject in a range of 1 to 100 mg per dose, such as a 20 mg per dose.

The term “adjuvant” refers to a substance or mixture that enhances the immune response to an antigen. Often, a primary immunization with an antigen alone, in the absence of an adjuvant, will fail to elicit an immune response.

The term “agonist” refers to a protein, nucleic acid, lipid, carbohydrate or chemical substance that interacts with a cellular receptor to produce a cellular response. Agonists that stimulate innate immune receptors may be of particular interest in the present technology.

The term “innate immune activator” is to be understood as referring to any substance that directly or indirectly activates a cell involved in the functioning of the innate immune system. Without limitation, innate immune activation may be manifest at the cellular level by one or more of changes in gene expression or protein production, induction of cytokine or chemokine production or secretion, changes in cell morphology, differentiation, cell division, changes in cell surface protein expression, chemotaxis, phagocytosis, exocytosis, autophagy, or apoptosis.

The term, “vaccine” is defined as an immuno-stimulatory treatment designed to elicit a beneficial immune response against a specific antigen, whether administered prophylactically or for the treatment of an already existing condition.

The term “immunogenic” refers to the ability of an antigen to elicit an immune response, including either humoral and/or cell-mediated immunity.

The term “immunologically-effective amount” as used herein in respect to an antigen or an innate immune activator refers to the amount of antigen or innate immune activator sufficient to elicit an immune response as measured by standard assays known to one skilled in the art. The effectiveness of an antigen as an immunogen, can be measured either by T-cell proliferation or cytokine secretion assays, by cytotoxicity assays, such as chromium release assays to measure the ability of a T-cell to lyse its specific target cell, or by measuring the levels of B-cell activity by measuring the levels of circulating antibodies specific for the antigen in serum, or by measuring the number of antibody spot-forming B cells, e.g., by ELISPOT. Furthermore, the level of protection of the immune response may be measured by challenging the immunized host with a replicating virus, pathogen or cell containing the antigen that has been immunized against. For example, if the antigen to which an immune response is desired is a virus or a tumor cell, the level of protection induced by the “immunogenically effective amount” of the antigen is measured by detecting the level of survival after virus or tumor cell challenge of the animals. Alternatively, protection can also be measured as the reduction in viral replication or tumor growth following challenge of the animals. The amount of antigen necessary to provide an immunogenic amount is readily determined by one of ordinary skill in the art, e.g., by preparing a series of vaccines of the technology with varying concentrations of antigen, administering the vaccine formulations to suitable laboratory animals (e.g., mice, rats, guinea pigs, or rabbits), and assaying the resulting immune response by measuring serum or mucosal antibody titers, antigen-induced swelling in the skin (delayed type hypersensitivity assay), T-cell proliferation, cytokine production or cytotoxic activity, protection against pathogen challenge and the like.

The term ‘parenteral’ refers to injection of a vaccine into any tissue of the body and includes intramuscular, subcutaneous, intradermal, intraperitoneal and intraocular routes of vaccine administration, by methods and delivery devices well known in the art.

In certain embodiments, the subject is a human. In other embodiments, the subject is animal, including but not limited to a dog, cat, horse, camel, cow, pig, sheep, goat, chicken, hawk, rabbit and fish. The term “animal” includes all domestic and wild mammals, fish, fowl, and includes, without limitation, cattle, horses, swine, sheep, goats, camels, dogs, cats, rabbits, deer, mink, chickens, ducks, geese, turkeys, game hens, and the like.

In certain embodiments, as an additional component, the composition of the technology may also optionally include an immunologically-effective amount of a chemical substance that activates one or more types of innate immune cell, such as a monocyte, dendritic cells, NK cell, lymphocyte or granulocyte. As known in the art, examples of chemicals that induce activation of innate immune cells include leukotrienes, prostaglandins, cytokines, chemokines, interferons, kinins, vitamin D, phorbol myristate acetate, ionomycin, mitogens, opsonins, histamine, bradykinin, serotonin, leukotrienes, cAMP, antimicrobial peptides, and pro-drugs or inducers of the aforementioned substances.

In certain embodiments, the PAMP innate immune activator of the current technology is an immunologically-effective amount of a substance that binds to an innate immune receptor. Currently known innate immune receptors include TLR-1, TLR-2, TLR-3, TLR-4, TLR-5, TLR-6, TLR-7, TLR-8, TLR-9, murine TLR-11; NOD-1, NOD-2, other NOD-like receptors (NLRs) such as NLRP1, NLRP3, NLRP12, NLRC4; DECTIN-1; DC-SIGN; AIM-2; mannose receptors including C-type lectins, MD2; CD14; LBP; CARD (caspase activating and recruitment domain)-containing proteins, such as RIG-1 (retinoic acid-inducible gene-1) and MDA-5 (melanoma differentiation-associated gene-5), LGP2 and ASC, scavenger receptors including CD-36, CD-68, and SRB-1, C reactive protein, mannose binding lectin, complement factors including C3a and C4b and complement receptors, and N-formyl Met receptors including FPR and FPRL1. Of particular interest for the present technology are PAMPs that bind and activate TLR-1, -2, -3, -5, -6, -7, and -9 and NOD-like receptors. More preferred are TLR3, TLR9 and NOD2 receptor agonists.

In certain embodiments, an immunologically-effective amount of one or more PAMPs (pathogen-associated molecular patterns) is/are used. A PAMP is a structurally conserved molecule derived from a pathogen that is immunologically distinguishable from host molecules, and is recognized by and specifically binds to an innate immune receptor. PAMPs are present in certain protein, lipid, lipoprotein, carbohydrate, glycolipid, glycoprotein, and nucleic acids expressed by particular pathogens and include TLR2 agonists including di- and tri-acyl lip peptides, lipotechoic acid, zymosan, peptidoglycan, poring, Lipoarabinomannan, Phospholipomannan, Glucuronoxylomannan, glycosylphosphatidylinositol (GPI)-anchored proteins in parasites, TLR3 agonists including double stranded RNA, including synthetic dsRNA for example polyinosinic:polycytidylic acid (poly I:C), TLR4 agonists including mannan, glucuronoxylomannan, heat shock protein, fibrinogen and synthetic MPL, TLR5 agonists including flagellin, TLR6 agonists including lipotechoic acid, TLR7 and TLR8 agonists including viral or synthetic single stranded (ss)RNA, for example, imiquimod and resiquimod (R848), and TLR9 agonists including unmethylated cytosine-guanine dinucleotide oligonucleotide sequences (CpG ODN) and hemozoin, RIG-1 agonists such as viral or synthetic double-stranded (ds) RNA, MDA5 agonists such as viral or synthetic dsDNA, NOD1 agonists including peptidoglycan containing the muramyl dipeptide NAG-NAM-gamma-D-glutamyl-meso diaminopimelic acid, NOD2 agonists including peptidoglycan containing the muramyl dipeptide NAG-NAM-L-alanyl-isoglutamine, RIG1 and MDA5 agonists including ssRNA and dsRNA, N-formyl Met receptor agonists including N-formyl methionine. Hence, a PAMP innate immune activator as used by the current technology may be selected from any of the above groups of agonists or synthetic analogues or derivatives thereof.

In certain embodiments, the substance comprising one or more pathogen-associated molecular pattern (PAMP) may be present, or administered, at an immunologically effective amount and/or concentration in the range of 0.01 to 500 micrograms per kilogram of body weight.

In certain embodiments, one or more of the substances comprising a pathogen-associated molecular pattern (PAMP) is present, or administered, as a pure, distinct and single molecular and chemical entity.

In certain embodiments, the substance comprising a pathogen-associated molecular pattern (PAMP) may be present, or administered, in a highly purified state, whereby one or more of each distinct and single molecular and chemical PAMP entity is at a purity of at least 50%, 60%, 70%, 80%, 90%, 95%, 96%, 97%, 98%, 99%, 99.9%, 99.99%, 99.999% or essentially 100%.

In certain embodiments, the PAMP of the current technology is a TLR agonist. There are presently believed to be approximately 10-15 different types of TLR in most mammalian species. The different TLRs bind and are activated by a range of natural and synthetic ligands. Different TLRs signal through different signaling molecules, although a feature in common is that they all activate the inflammatory transcription factor NFκB.

In certain embodiments, the PAMP of the current technology is a TLR1 agonist, such as a TLR1 agonist drawn from the group of a triacyl lipopeptide and Pam3CSK4.

In certain embodiments, the PAMP of the current technology is a TLR2 agonist, such as a TLR2 agonist drawn from the group of a glycolipid, lipoteichoic acid, peptidoglycan, HSP70, zymosan, and Pam3CSK4.

In certain embodiments, the PAMP of the current technology is a TLR3 agonist, such as a TLR3 agonist drawn from the group of a double-stranded RNA, poly (I:C), poly (I:C-LC) (Hiltonol™), and poly I:polyC12 U (Ampligen™)

In certain embodiments, the PAMP of the current technology is a TLR4 agonist, such as a TLR4 agonist drawn from the group of monophosphoryl lipid A (MPLA), heat shock proteins, fibrinogen, heparan sulfate fragments, hyaluronic acid fragments, and synthetic TLR4 agonists including E6020, GLA and LPS peptide mimotopes. Most preferred is a synthetic TLR4 agonist that preferentially signals through the TIR-domain-containing adapter-inducing interferon-β (TRIF) and not the NFκB pathway. Due to toxicity and regulatory requirements, lipopolysaccharide (LPS) TLR4 agonists and substances containing LPS (such as endotoxin) should be avoided in the technology. The amount of LPS and/or endotoxin in substances and compositions used in the aspects of the present technology may be less than 100 EU per dose, such as less than 90, 80, 70, 60, 50 40, 30, 20, 10, 5, 4, 3, 2, 1 or less EU per dose. The concentration of LPS and/or endotoxin in substances and compositions used in the aspects of the present technology may be less than 200 EU/m³, such as less than 150, 100, 90, 80, 70, 60, 50 40, 30, 20, 10, 5, 4, 3, 2, 1 or less EU/m³

In certain embodiments, the PAMP of the current technology is a TLR5 agonist, such as a TLR5 agonist drawn from the group of bacterial or synthetic flagellins.

In certain embodiments, the PAMP of the current technology is a TLR6 agonist, such as a TLR6 agonist drawn from the group of diacyl lipopeptides. Most preferred is diacyl lipopeptide.

In certain embodiments, the PAMP of the current technology is a TLR7 agonist, such as a TLR7 agonist drawn from the group of viral single-stranded RNA, imidazoquinoline, gardiquimod, loxoribine, bropirimine, CL264, R848, and CL075. Most preferred is R848.

In certain embodiments, the PAMP of the current technology is a TLR8 agonist, such as a TLR8 agonist drawn from the group of single-stranded RNA, PolyU, imiquimod, resiquimod, ssPolyU/LyoVec and ssRNA40/LyoVec.

In certain embodiments, the PAMP of the current technology is a TLR9 agonist. More preferably, the TLR9 agonist is a CpG ODN. The term “CpG” or “CpG ODN molecule”, as used herein, is to be understood as referring to a ODN molecule comprising a motif wherein a cytosine nucleoside is followed by a guanine nucleoside, linked by a phosphate molecule in the normal manner seen in polynucleotide sequences (i.e. a “CpG motif”), wherein the cytosine nucleoside is unmethylated. CpG motifs are prevalent in bacterial and viral genomes, but are rare in vertebrate genomes. Further, CpG motifs are generally unmethylated in prokaryotic organisms, whereas in eukaryotic organisms, DNA methyltransferases generally methylate 70-80% of the CpG motifs present. It also refers to a synthesized oligonucleotide molecule comprising at least one unmethylated CpG motif. Frequently, more than one CpG motif is present. A variety of CpG oligonucleotide molecules are commercially available. They are typically between 18-24 nucleotides in length, but a person skilled in the art will appreciate that CpG oligonucleotide molecules of other lengths are also suitable. The CpG oligonucleotide molecules can comprise various nucleotide sequences surrounding at least one CpG motif, as different nucleotide sequences have been shown to stimulate TLR9 to varying degrees. Class B ODN are strong stimulators of human B cell and monocyte maturation. They also stimulate the maturation of plasmacytoid dendritic cells (pDC) but to a lesser extent than Class A ODN and induce only very small amounts of IFNα. Class C ODN have features of both Class A and Class B ODN. Preferably a Class B or Class C CpG ODN is used in the current technology. As known to those skilled in the art, the CpG backbone can be varied from a natural phosphodiester backbone to a synthetic phosphorothioate backbone or a mixture of the two types of backbones to increase the stability of the ODN. In a preferred embodiment of the technology, the CpG PAMP has a natural phosphorothioate backbone and is 18 to 28 nucleotides in length. In another embodiment of the technology, the TLR9 agonist is a Class B or C CpG ODN with a synthetic phosphorothioate backbone and is 18 to 28 nucleotides in length. In another embodiment, the PAMP is drawn from the group of CpG2006, CpG1826 and, in another embodiment, CpG7909.

In certain embodiments, the PAMP of the current technology is a NOD-like receptor agonist. In certain embodiments, the agonist is to the NOD1 receptor and is drawn from the group of, Acylated derivative of iE-DAP) (C12-iE-DAP), D-gamma-Glu-mDAP (iE-DAP), L-Ala-gamma-D-Glu-mDAP (Tri-DAP). In certain embodiments, the agonist is to the NOD2 receptor and is drawn from the group of muramyl dipeptide (MDP), muramyl tripeptide, L18-MDP, M-TriDAP, murabutide, PGN-ECndi, PGN-ECndss, N-glycolylated muramyldipeptide, and PGN-Sandi. In certain embodiments, the NOD2 agonist is murabutide.

In certain embodiments, the PAMP of the current technology is an agonist of a C-type lectin receptor. In another embodiment, the C-type lectin receptor agonist binds to one of the group of macrophage mannose receptor, CLEC-2, DEC205/CD205, DC-SIGN-like, DC-ASGPR (MGL)/CD301, Dectin-1, Langerin/CD207, Mincle and CLR BDCA-2/CD303. In certain embodiments, the C-type lectin receptor agonist is drawn from the group of Beta-1,3-glucan, zymosan, Heat-killed C. albicans, cord factor, and Trehalose-6,6-dibehenate.

In certain embodiments, the PAMP of the current technology is an agonist of nucleotide-binding oligomerization domain-like receptor family (NLR) proteins including the retinoic acid inducible gene-based-1-like helicase receptor family that include RIG-1 and MDA-5. Preferably, it is drawn from the group of poly(I:C), Poly(dA:dT), Poly(dG:dC) and 5′ppp-dsRNA.

In certain embodiments, the PAMP of the current technology is an agonist of a DNA sensing protein drawn from the group of DNA-dependent activator of interferon-regulatory factors (DAI) and absent in melanoma 2 (AIM2), for example, Poly(dA:dT).

In certain embodiments, the PAMP of the current technology is an agonist of a class A, B or C scavenger receptor expressed on innate immune cells, which may, for example, be drawn from the group of SCARA1, SCARA2, SCARA3, SCARA4, SCARA5, SCARB1, SCARB2, SCARB3, MARCO, CD36, SR-B1, CD68, and LOX-1, e.g., low-density lipoprotein (LDL), oxidized LDL, acetylated LDL or chemically modified LDL.

In certain embodiments, the PAMP of the current technology is an agonist of NLRP1 or NALP3, e.g., hemozoin or ATP.

The selected PAMP innate immune activator of the current technology can be added to the substances and composition used in the aspects of the present technology in an “immunologically-effective” immunopotentiating amount which, as known to those of ordinary skill in the art, may vary depending on the species, strain, age, weight and sex of the animal or human being treated with the immunological composition.

The term “immunopotentiating amount” refers to the amount of an immunological formulation needed to effect an increase in immune response, as measured by standard assays known to one skilled in the art. As can be appreciated, each immunological formulation containing inulin particles (or other equivalent anti-inflammatory component) may have an effective dose range that may differ depending on the PAMP innate immune activator and specific inulin polymorphic form (or other equivalent anti-inflammatory component) used. Thus, a single dose range cannot be prescribed which will have a precise fit for each possible inulin particle (or other equivalent anti-inflammatory component) and PAMP innate immune activator combination within the scope of this technology. However, the immunopotentiating amount may easily be determined by one of ordinary skill in the art. The effectiveness of immune activation can be measured either by an immune cell proliferation assay, or assays measuring changes in the level of expression of cell surface activation markers, for example, by flow cytometry or fluorescent microscopy, or cytolytic assays, or by measuring the secretion of cytokines or chemokines or other substances secreted by activated immune cells, or by measuring activation-induced changes in immune cell gene expression, for example by real time polymerase chain reaction or gene expression arrays. The amount of each component of the immunological formulation necessary to provide an immunologically-effective amount is readily determined by one of ordinary skill in the art, e.g., by preparing a series of immunological formulations of the technology with varying concentrations of PAMP innate immune activator and inulin particles (or other equivalent anti-inflammatory component) then adding these formulations to cultures of immune cells and assaying immune cell activation by means known to one skilled in the art, including the assays detailed herein. Similarly, the amount of each component necessary to provide enhancement of the immune response to a vaccine antigen can be readily determined by one of ordinary skill in the art, for example, by preparing a series of immunological formulations of the technology with varying concentrations of PAMP innate immune activator and inulin particles (or other equivalent anti-inflammatory component) plus a vaccine antigen and administering the vaccine together with inulin particles (or other equivalent anti-inflammatory component), to suitable laboratory animals (e.g., mice or guinea pigs), and then assaying the resulting antigen-specific immune response by measurement of antigen-specific serum or mucosal antibody titers, antigen-induced swelling in the skin (DTH), or antigen-stimulated T-cell proliferation or cytokine production.

PAMP innate immune activators used in the technology can be effective in any animal, preferably a mammal, and most preferably a human. Different PAMP innate immune activators can cause optimal immune stimulation depending on the species. Thus a PAMP immune activator such as a specific CpG ODN that provides optimal stimulation in humans by binding to human TLR9 may not cause optimal stimulation in a mouse expressing mouse TLR9, or vice versa. One of ordinary skill in the art can identify the optimal PAMP innate immune activators useful for a particular species of interest using routine immune assays described herein or known in the art.

The aqueous portion of the compositions and substances of the aspects of the present technology may be buffered in iso-osmotic saline. Because the compositions and substances may be intended for parenteral or mucosal administration, it may be appropriate to formulate these solutions so that the tonicity is essentially the same as normal physiological fluids in order to prevent post-administration swelling or rapid absorption of the composition due to differential ion concentrations between the composition and physiological fluids. It may also be appropriate to buffer the saline in order to maintain a pH compatible with normal physiological conditions. For example, the buffered pH may suitably be in the range of 4 to 10, in the range 5 to 9, in the range 6 to 8.5, or in the range 7 to 8.5. Also, in certain instances, it may be necessary to maintain the pH at a particular level in order to insure the stability of certain composition components, such as the inulin particles, PAMP or the protein antigens in a formulation. Any physiologically acceptable buffer may be used herein, but it has been found that it is most convenient to use bicarbonate buffered saline (1%) at a pH of between 6 and 8.5. Suitable preservatives include benzalkonium chloride (0.003-0.03% w/v); chlorobutanol (0.3-0.9% w/v); parabens (0.01-0.25% w/v) and thimerosal (0.004-0.02% w/v).

The technology in other aspects includes a method of modulating including inducing or suppressing a non-antigen-specific immune response. In one aspect, the present technology provides a method of enhancing protection against a pathogen, wherein said method comprises administering to a subject a therapeutically effective amount of the compositions or substances of the technology. This may provide temporary protection against various pathogens including viruses, bacteria, parasites, fungi and protozoa, for treatment of cancer, or prevention or treatment of autoimmune disease, asthma or allergy. The method involves the steps of administering to a subject the immunological composition of the present technology in an immunologically-effective amount. For longer-term protection, the immunological composition may be administered more than once.

In various embodiments, the immunological composition of the technology is intended for treatment or prevention of a variety of diseases. Thus, in various embodiments, the immunological composition is provided in an amount effective to treat or prevent an infectious disease, a cancer, or an allergy. Accordingly, the methods provided herein can be used on a subject that has or is at risk of developing an infectious disease and therefore the method is a method of treating or preventing the infectious disease. The methods can also be used on a subject that has or is at risk of developing asthma and the method is a method of treating or preventing asthma in the subject. The method can also be used on a subject that has or is at risk of developing allergy and the method is a method of treating or preventing allergy. The method can also be used on a subject that has or is at risk of developing a cancer and the method is a method of treating or preventing the cancer.

The compositions and substances used in the aspects of the present technology may be used in some embodiments to alter the type or magnitude of the immune response including in one option to a co-administered antigen. Accordingly, it is proposed that the compositions and substances can be widely used as a vaccine adjuvant, for example, by combining it/them with one or more relevant antigens to form a prophylactic or therapeutic vaccine. Thus, in certain embodiments, the compositions and substances of the aspects of the present technology further comprise a vaccine antigen. Alternatively, the subject to be treated is further administered a vaccine antigen at the same time as or following the administration of an immunologically effective amount of the immunological composition. In various embodiments, the antigen may one or more of a microbial antigen, a self-antigen, a cancer antigen, and an allergen, but it is not so limited. In various embodiments, the microbial antigen is one or more of a bacterial antigen, a viral antigen, a fungal antigen and a parasitic antigen. In another embodiment, the antigen is a peptide antigen. In another embodiment, the antigen is encoded by a nucleic acid vector. In another embodiment, the composition further comprises a cytokine, or the subject is further administered a cytokine.

The term “antigen” refers to any substance, usually a protein or glycoprotein, lipoprotein, saccharide, polysaccharide or lipopolysaccharide, which upon administration stimulates the formation of specific antibodies or memory T cells. An antigen can stimulate the proliferation of T-lymphocytes with receptors for the antigen, and can react with the lymphocytes to initiate the series of responses designated cell-mediated immunity.

Suitable antigens for use in this technology include substances from microbes (bacteria, fungi, protozoa, or viruses) or endogenous substances against which a specific immune response can be generated. Antigens may be prepared from inactivated organisms or may be generated by recombinant protein technology or directly synthesized. For the purposes of this description, an antigen is defined as any protein, carbohydrate, lipid, nucleic acid, or mixture of these, or a plurality of these, to which an immune response is desired. The term antigen as used herein also includes combinations of haptens with a carrier. A hapten is a portion of an antigenic molecule or antigenic complex that determines its immunological specificity, but is not sufficient to stimulate an immune response in the absence of a carrier. Commonly, a hapten is a relatively small peptide or polysaccharide and may be a fragment of a naturally occurring antigen. A hapten will react specifically in vivo and in vitro with homologous antibodies or T-lymphocytes. Haptens are typically attached to a large carrier molecule such as tetanus toxoid or keyhole limpet hemocyanin (KLH) by either covalent or non-covalent binding before formulation as a vaccine.

Antigens can be used in vaccines to either treat or prevent a disease. They can also be used to generate specific immune substances, such as antibodies, which can be used in diagnostic tests or kits. The subjects of an antigen-containing vaccine are typically vertebrates, preferably a mammal, more preferably a human. It is not always necessary that the antigen be identified in molecular terms. For example, immune responses to tumors can be generated without knowing either in advance or post-hoc which molecules the immune response is directed against. In these cases, the term antigen refers to the substance or substances, known or not known, toward which a specific immune response is directed. The specificity of the immune response provides an operational definition of an antigen, such that immunity generated against one type of tumor may be specific for that tumor type but not another tumor type.

In one embodiment, the encoded antigen may be derived from a virus such as influenza, including inactivated influenza virus or influenza haemagglutinin, neuraminidase or M2 protein or other components of the influenza virus. Examples of other RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus, the genus Apthovirus (Foot and Mouth disease; the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesvirises (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). DNA viruses also include Kuru and Creutzfeldt-Jacob disease viruses and chronic infectious neuropathic agents (CHINA virus). Each of the foregoing lists is illustrative, and is not intended to be limiting.

Other examples of antigens suitable for the technology include, but are not limited to, infectious disease antigens for which a protective immune response may be desired including the human immunogenicity virus (HIV) antigens gag, env, pol, tat, rev, nef, reverse transcriptase, and other HIV components or a part thereof, the E6 and E7 proteins from human papilloma virus, the EBNA1 antigen from herpes simplex virus, hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin, neuraminidase, nucleoprotein, M2, and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegalovirus antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS1, NS 1, NS 1-NS2A; rabies viral antigens such as rabies glycoprotein, rabies nucleoprotein and other rabies viral antigen components; West Nile virus prM and E proteins; and Ebola envelope protein. See Fundamental Virology, Second Edition, eds. Knipe, D. M. and, Howley P. M. (Lippincott Williams & Wilkins, New York, 2001) for additional examples of viral antigens. In addition, bacterial antigens are also disclosed. Bacterial antigens which can be used in the compositions and methods of the technology include, but are not limited to, pertussis bacterial antigens such as pertussis toxin, filamentous hemagglutinin, pertactin, FIM2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; Staphylococcal bacterial antigens such as IsdA, IsdB, SdrD, and SdrE; gram-negative bacilli bacterial antigens such as lipopolysaccharides, flagellin, and other gram-negative bacterial antigen components; Mycobacterium tuberculosis bacterial antigens such as mycolic acid, heat shock protein 65 (HSP65), the 30 kDa major secreted protein, antigen 85A, ESAT-6, and other mycobacterial antigen components; Helicobacter pylori bacterial antigen components; pneumococcal bacterial antigens such as pneumolysin, pneumococcal capsular polysaccharides and other pneumococcal bacterial antigen components; haemophilus influenza bacterial antigens such as capsular polysaccharides and other haemophilus influenza bacterial antigen components; anthrax bacterial antigens such as anthrax protective antigen, anthrax lethal factor, and other anthrax bacterial antigen components; the F1 and V proteins from Yersinia pestis; rickettsiae bacterial antigens such as romps and other rickettsiae bacterial antigen components. Also included with the bacterial antigens described herein are any other bacterial, mycobacterial, mycoplasmal, rickettsial, or chlamydial antigens. Examples of protozoa and other parasitic antigens include, but are not limited to, plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components. Examples of fungal antigens include, but are not limited to, antigens from Candida species, Aspergillus species, Blastomyces species, Histoplasma species, Coccidiodomycosis species, Malassezia furfur and other species, Exophiala werneckii and other species, Piedraia hortai and other species, Trichosporum beigelii and other species, Microsporum species, Trichophyton species, Epidermophyton species, Sporothrix schenckii and other species, Fonsecaea pedrosoi and other species, Wangiella dermatitidis and other species, Pseudallescheria boydii and other species, Madurella grisea and other species, Rhizopus species, Absidia species, and Mucor species. Examples of prion disease antigens include PrP, beta-amyloid, and other prion-associated proteins.

In addition to the use of the compositions and substances of the aspects of the present technology to induce an antigen specific immune response in humans, the methods of certain embodiments are particularly well suited for treatment of horses and other animals. The methods of the technology can be used to protect against infection in livestock, including cows, camels, horses, pigs, sheep, and goats. Horses are susceptible to flaviviruses including Japanese encephalitis and West Nile virus. In certain embodiments, the immunological composition of the technology can be administered to horses together with inactivated Japanese encephalitis virus antigen to protect them against Japanese encephalitis and related flaviviruses.

In addition to the infectious and parasitic agents mentioned above, another area for desirable enhanced immunogenicity to a non-infectious agent is in the area of cancer, in which cells expressing cancer antigens are desirably eliminated from the body. A “cancer antigen” as used herein is a compound, such as a peptide or protein, present in a tumor or cancer cell and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared by recombinant DNA expression technology or by any other means known in the art. In one embodiment, the cancer is chosen from biliary tract cancer; bone cancer; brain and CNS cancer; breast cancer; cervical cancer; choriocarcinoma; colon cancer; connective tissue cancer; endometrial cancer; esophageal cancer; eye cancer; gastric cancer; Hodgkin's lymphoma; intraepithelial neoplasms; larynx cancer; lymphomas; liver cancer; lung cancer (e.g., small cell and non-small cell); melanoma; neuroblastomas; oral cavity cancer; ovarian cancer; pancreas cancer; prostate cancer; rectal cancer; sarcomas; skin cancer; testicular cancer; thyroid cancer; and renal cancer. Cancer antigens which can be used in the compositions and methods of the technology include, but are not limited to, prostate specific antigen (PSA), breast, ovarian, testicular, melanoma, telomerase; multidrug resistance proteins such as P-glycoprotein; MAGE-1, alpha fetoprotein, carcinoembryonic antigen, mutant p53, papillomavirus antigens, gangliosides or other carbohydrate-containing components of melanoma or other cancer cells. It is contemplated by the technology that antigens from any type of cancer cell can be used in the compositions and methods described herein. The antigen may be a cancer cell, or immunogenic materials isolated from a cancer cell, such as membrane proteins. Included are survivin and telomerase universal antigens and the MAGE family of cancer testis antigens.

In another embodiment, the compositions and methods of the technology include antigens involved in autoimmunity that can be used to induce immune tolerance. Such antigens include, but are not limited to, myelin basic protein, myelin oligodendrocyte glycoprotein and proteolipid protein of multiple sclerosis, CII collagen protein of rheumatoid arthritis, glutamic acid decarboxylase, insulin and tyrosine phosphatase proteins of type 1 diabetes mellitus, gliadin protein of celiac disease.

In another embodiment, the compositions, substances and methods of the aspects of the present technology can be used with antigens known as “allergens” involved in allergy to induce tolerance and suppress allergen-specific IgE. An “allergen” is any substance that can induce an allergic or asthmatic response in a susceptible subject. Allergens include pollens, insect venoms, animal dander dust, fungal spores and drugs (e.g., penicillin). Examples of natural, animal and plant allergens include but are not limited to proteins specific to the following genuses: Canine (Canis familiaris); Dermatophagoides (e.g., Dermatophagoides farinae); Felis (Felis domesticus); Ambrosia (Ambrosia artemiisfolia; Lolium (e.g., Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g., Plantago lanceolata); Parietaria (e.g., Parietaria officinalis or Parietaria judaica); Blattella (e.g., Blattella germanica); Apis (e.g., Apis multiflorum); Cupressus (e.g., Cupressus sempervirens, Cupressus arizonica and Cupressus macrocarpa); Juniperus (e.g., Juniperus sabinoides, Juniperus virginiana, Juniperus communis and Juniperus ashei); Thuya (e.g., Thuya orientalis); Chamaecyparis (e.g., Chamaecyparis obtusa); Periplaneta (e.g., Periplaneta americana); Agropyron (e.g., Agropyron repens); Secale (e.g., Secale cereale); Triticum (e.g., Triticum aestivum); Dactylis (e.g., Dactylis glomerata); Festuca (e.g., Festuca elatior); Poa (e.g., Poa pratensis or Poa compressa); Avena (e.g., Avena sativa); Holcus (e.g., Holcus lanatus); Anthoxanthum (e.g., Anthoxanthum odoratum); Arrhenatherum (e.g., Parrhenatherum elatius); Agrostis (e.g., Agrostis alba); Phleum (e.g., Phleum pratense); Phalaris (e.g., Phalaris arundinacea); Paspalum (e.g., Paspalum notatum); Sorghum (e.g., Sorghum halepensis); and Bromus (e.g., Bromus inermis).

In another embodiment, the compositions, substances and methods of the aspects of the present technology can be used to immunize against antigens involved in asthma. Such antigens include, but are not limited to IgE and histamine.

The term “treatment” as used herein covers any treatment of a disease in a bird, fish or mammal, particularly a human, and includes:

(i) preventing the disease from occurring in a subject which may be predisposed to the disease but has not yet been diagnosed as having it;

(ii) inhibiting the disease, i.e., slowing or arresting its development; or

(iii) relieving the disease, i.e., causing regression of the disease. (It should be noted that vaccination may effect regression of a disease where the disease persists due to ineffective antigen recognition by the subject's immune system, where the vaccine effectively presents antigen.)

The term “optionally” means that the subsequently described event or circumstances may or may not occur, and that the description includes instances where said event or circumstances occurs and instances in which it does not occur.

The term “modulation of the immune response” is to be understood as the induction of any induced change in an immune cell, which can be measured in a manner known to those of ordinary skill in the art. Preferably, the measured parameter to indicate a change in the behavior or function of immune cells will be selected from the group of a change in gene expression, protein expression, cell morphology, differentiation, cell division, cell surface protein expression, chemotaxis, phagocytosis, exocytosis, autophagy, chemokine secretion, cytokine secretion and apoptosis.

In a further embodiment of the technology, the co-administration of an inulin particle (or other equivalent anti-inflammatory component) with a PAMP innate immune activator allows dose-sparing of the PAMP innate immune activator. Hence in the presence of a inulin particle (or other equivalent anti-inflammatory component), a lower dose of a PAMP innate immune activator can be used to obtain the same level of immune activation. Given the different actions of a inulin particle (or other equivalent anti-inflammatory component) and a PAMP innate immune activator, the dose-sparing effect of inulin particles (or other equivalent anti-inflammatory component) allows a lower dose of PAMP immune activator to be used to achieve a desired immune response or adjuvant effect and thereby provides a means to reduce any dose-related side effects or toxicity of the PAMP innate immune activator, while still achieving the desired immune outcome. As dose-related toxicity from excess PAMP innate immune activation and inflammation are the main dose-limiting side effects of PAMP innate immune activators, the technology provides a novel means to reduce the dose-related side effects of PAMP innate immune activators.

The composition and substances of the present technology may optionally be administered in its/their separate components simultaneously or sequentially but preferably the inulin particle component (or other equivalent anti-inflammatory component) is administered together with or prior to the antigen rather than following the antigen. When the components of the composition or substances of the aspects of the present technology are administered simultaneously they can be administered in the same or separate formulations, and in the latter case at the same or separate injection sites, and at the same time as the vaccine antigen. The PAMP innate immune activator component can be administered before, after, or simultaneously with the inulin particles (or other equivalent anti-inflammatory component) and the antigen component. For instance, the PAMP innate immune activator component may be administered prior to or after the administration of the inulin particle (or other equivalent anti-inflammatory component) component together with a priming dose of antigen. The boost dose of antigen may subsequently be administered with either or both of the PAMP innate immune activator and the inulin particle component (or other equivalent anti-inflammatory component). A “prime dose” is the first dose of antigen administered to the subject. A “boost dose” is a second, third, or subsequent dose of antigen administered to a subject that has already been exposed to the antigen. Where the components are administered sequentially, the separation in time between the administrations of the components may be a matter of minutes or longer. In various embodiments, the separation in time is less than 7 days, 3 days, 2 days or less than 1 day.

The compositions or substances of the present technology may be used to enhance a vaccine response in association with use of a DNA vaccine. In certain embodiments, the compositions or substances of the aspects of the present technology with a protein or other physical antigen is/are administered as a boost dose following one or more prime doses of an effective immunogenic amount of a DNA vaccine encoding one or more antigens. In a further embodiment, the composition or substances of the aspects of the present technology is/are administered with a protein or other physical antigen at the same time as a DNA vaccine encoding one or more antigens is administered either at a different injection site or mixed together and administered at the same injection site.

The compositions or substances of the present technology with or without the addition of a physical antigen may also be administered together with a vector encoding an antigen. In its broadest sense, a “vector” is any vehicle capable of facilitating the transfer to and expression by the infected cell of an encoded or enclosed antigen. In general, the vectors useful in the technology include, but are not limited to, plasmids, phages, viruses, and other vehicles derived from viral or bacterial sources that have been manipulated by the insertion or incorporation of the antigen nucleic acid sequences. Viral vectors are a preferred type of vector and include, but are not limited to, nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; retrovirus; lentivirus and sendai virus. It is known in the art how to readily employ other vectors in a similar fashion to deliver antigens to cells. See, e.g., Sanbrook et al., “Molecular Cloning: A Laboratory Manual,” Second Edition, Cold Spring Harbor Laboratory Press, 1989.

One or more of the preparations of the compositions substances of the present technology may include an antigen-binding carrier material or allergen-binding carrier material. The antigen-binding carrier material or allergen-binding carrier material may comprise, for example, one or more metal salts such as aluminum hydroxide, aluminum phosphate, aluminum sulphate, calcium phosphate, calcium sulphate, ferrous and ferric phosphate, ferrous and ferric sulphate, chromium phosphate and chromium sulphate. Other suitable antigen-binding carrier materials and allergen-binding carrier materials include proteins, lipids and carbohydrates (e.g., heparin, dextran and cellulose derivatives), and organic bases such as chitin (poly N-acetylglucosamine) and deacetylated derivatives thereof, as known to those of ordinary skill in the art.

In certain embodiments, the PAMP innate immune activator in the immunological composition is physically bound to the inulin particle (or other equivalent anti-inflammatory component) or to the antigen-binding carrier material incorporated with the inulin particle (or other equivalent anti-inflammatory component). In certain embodiments, the PAMP innate immune activator is bound to the inulin particle (or other equivalent anti-inflammatory component) by a bond selected chosen from covalent, hydrostatic, and electrostatic bonds. Alternatively, the PAMP innate immune activator can be sterically trapped inside the inulin particle (or other equivalent anti-inflammatory component). In certain embodiments, a linker sequence can be used to join the PAMP innate immune activator to the inulin particle (or other equivalent anti-inflammatory component).

Further, where the compositions or substances of the present technology include an antigen-binding material, in certain embodiments the inulin particles (or other equivalent anti-inflammatory component) are combined with or bound to the antigen-binding carrier material. Co-crystals of inulin particles and an antigen-binding carrier material may be prepared by, for example, a method comprising:

(a) preparing a suspension of the inulin particles;

(b) heating the suspension until the inulin particles dissolve;

(c) adding to said solution an amount of an antigen-binding carrier material;

(d) re-precipitating the inulin particles from said suspension; and

(e) isolating formed particles comprising inulin particles and one or more antigen-binding carrier material

In a development of this work, the inulin particles can be formulated with an antigen-binding carrier material, in particular, aluminum hydroxide or aluminum phosphate (collectively referred to as “alum”) gel. Alum gel has been widely used as an adjuvant in vaccines wherein it is known to induce a strong antibody (Th2) immune response but only a poor cellular (Th1) immune response. Thus, it has been found possible to form co-crystallized particles of gIN, dIN or eIN together with aluminum salts (for example aluminum hydroxide or aluminum phosphate), to form, respectively, a gIN/alum preparation (also referred to as “Algammulin”) (see WO 90/01949, WO 2006/024100), a dIN/alum preparation (also referred to as “Aldeltin”) or an eIN/alum hydroxide preparation (also referred to as “Alepsilin”). While in vivo studies have shown that vaccines containing complexes of inulin particles and aluminum salts are well tolerated, their ability to increase antibody responses to co-administered antigens over and above the inulin particle or alum adjuvant formulation alone are generally modest and additive rather than synergistic, and like alum adjuvants alone, the formulation of inulin with alum biases the resultant immune response towards a Th2 rather than a Th1 response. This may not be desirable for particular vaccines where it is sought to induce Th1 immunity to a co-administered antigen. In particular, without wishing to be restricted by theory, adjuvants that enhance Th1 immunity tend to inhibit the magnitude of a Th2 response and vice versa, via a complex array of feedback pathways involving factors such as the Th1 cytokine IFN-γ, which inhibits Th2 responses, whereas the Th2 cytokines, IL-4 and IL-10, inhibit Th1 responses. A bias towards a Th2 response may be undesirable if it means that less of a Th1 response can be achieved and vice versa. In one embodiment of this technology, it has been found that the Th2 bias seen when inulin is co-crystallized with aluminum salts, as in the case of Algammulin, Aldeltin or Alepsilin, or phosgammulin, phosdeltin or phosepsilin is reduced or no longer evident when the inulin particle-alum particles are combined with a PAMP innate immune activator. Conversely, the strong Th1 bias often observed with some innate immune activators alone, for example with TLR9 agonists, is reduced or no longer evident when TLR9 agonists are formulated with inulin particles with or without an antigen-binding alum. In the presence of inulin particles, both Th1 and Th2 immune responses develop in parallel, resulting in an improved immune response against a co-administered antigen not achievable with use of the individual components alone. The inulin particle (or other equivalent anti-inflammatory component) combined with the antigen-binding carrier material may comprise a relative amount by weight of the inulin (or other equivalent anti-inflammatory component) to the antigen-binding carrier material in the range of 1:20 to 200:1, such as 1:5 to 50:1, or 1:2 to 20:1.

In another embodiment, the compositions or substances according to the present technology may further comprise a therapeutic agent such as an anti-microbial agent, an anti-cancer agent, and an allergy or asthma medicament, or the subject is further administered a therapeutic agent selected from the same group. In a related embodiment, the anti-microbial agent is one or more of an anti-bacterial agent, an anti-viral agent, an anti-fungal agent, or an anti-parasite agent.

In a related embodiment, the anti-cancer agent included with the immunological composition is one or more of a chemotherapeutic agent, a cancer vaccine, or an immunotherapeutic agent.

In a related embodiment, the allergy or asthma medicament included with the immunological composition is one or more of PDE-4 inhibitor, bronchodilator/beta-2 agonist, K+ channel opener, VLA-4 antagonist, neurokin antagonist, TXA2 synthesis inhibitor, xanthanine, arachidonic acid antagonist, 5 lipoxygenase inhibitor, thromboxin A2 receptor antagonist, thromboxane A2 antagonist, inhibitor of 5-lipox activation protein, or protease inhibitor.

The compositions or substances of the present technology may be formulated for parenteral administration or may be formulated in a sustained release device. The sustained release device may be a microparticle, a matrix or an implantable pump, but it is not so limited.

In another embodiment, the compositions and substances of the aspects of the present technology is/are formulated for delivery to a mucosal surface. In related embodiments, the compositions and substances of the aspects of the present technology is/are provided in an amount effective to stimulate a mucosal immune response. The mucosal surface may be an oral, nasal, rectal, vaginal, and ocular surface, but is not so limited. In one embodiment, the compositions and substances of the present technology is/are formulated for oral administration.

The compositions and substances of the present technology may also be formulated as a nutritional supplement. In a related embodiment, the nutritional supplement is formulated as a capsule, a pill, or a sublingual tablet. In another embodiment, the immunological composition is formulated for local administration.

In embodiments relating to the treatment of a subject, the method or use may further comprise isolating an immune cell from the subject, contacting the immune cell with an immunologically-effective amount of the compositions and substances of the aspects of the present technology to thereby produce an ex vivo activated immune cell; and optionally then re-administering the activated immune cell to the subject. In one embodiment, the immune cell is a monocyte and in another embodiment the immune cell is a dendritic cell. In another embodiment, the method or use may further comprise contacting the immune cell with an antigen in the presence of, before or after the addition of an immunologically-effective amount of the compositions or substances of the aspects of the present technology

In still another aspect, the technology provides a method of identifying an optimal immunological composition by measuring a control level of activation of an immune cell population contacted with a composition or substances of the aspects of the present technology, then comparing this with the level of activation of an immune cell population contacted with a test composition, wherein a test level that is equal to or above the control level is indicative of a suitable immunological composition.

The immune response may comprise immune activation as manifest by changes in gene expression or protein production such as induction of cytokine or chemokine production or secretion, changes in phenotype, proliferative or survival capacity or modulation of immune effector properties. The immune response may further comprise induction, enhancement or modulation of an adaptive immune response with induction of antibody production or induction of a T-cell effector or memory response against an endogenous or exogenous antigen.

In a further aspect, the present technology provides a method of modulating an immune response, wherein said method comprises administering to a subject a therapeutically effective amount of the compositions or substances of the aspects of the present technology.

As used herein, the term “effective amount” refers to a non-toxic but sufficient amount of the compositions and substances of the aspects of the present technology to provide the desired effect. The exact amount required will vary from subject to subject depending on factors such as the species being treated, the age and general condition of the subject, the severity of the condition being treated, the particular composition or substances of the aspects of the present technology being administered and the mode of administration. Thus, it is not possible to specify an exact “effective amount”. However, for any given case, an appropriate “effective amount” may be routinely determined by persons of ordinary skill in the art.

In certain embodiments, the technology further provides a method of modulating the patterns of cytokines produced in response to a vaccine. The term “modulate” envisions the suppression of expression of a particular cytokine when lower levels are desired, or augmentation of the expression of a particular cytokine when higher levels are desired. Modulation of a particular cytokine can occur locally or systemically. PAMP innate immune activators used as vaccine adjuvants can directly activate macrophages and dendritic cells to secrete cytokines such as TNF-α and IL-1. Cytokine profiles induced by PAMPs innate immune activators determine T-cell regulatory and effector functions in immune responses and may also contribute to vaccine adverse reactions. In general, PAMP innate immune activators induce cytokines associated with inflammation and fever including TNF and IL-1, but may also induce suppressive cytokines such as IL-10, which provide inhibitory feedback and may thereby limit or inhibit the adaptive immune response to a co-administered antigen. The compositions and substances of the aspects of the present technology is/are able to modulate the cytokines induced by a PAMP innate immune activator, and thereby lead to a more favorable immune response.

In other aspects the technology includes a method of preventing in a subject excess polarization of the immune response otherwise caused by administering to the subject a combination of an antigen and a PAMP innate immune activator such as a TLR agonist. It has been previously shown that the combination of a PAMP innate immune activator such as CpG ODN, a TLR9 agonist, resulted in a Th1 bias and suppression of the Th2 arm of the response. It was thus a surprising finding that when inulin particles are combined with a Th1-biasing PAMP innate immune activator such as CpG ODN, it is possible to maintain a strong Th2 response while at the same time also inducing a Th1 immune response to a co-administered antigen, thereby resulting in a synergistic increase in both the Th2 and Th1 response to the antigen, to an extent that the components in the absence of the inulin particles could not produce.

The compositions and substances of the present technology may be formulated in a pharmaceutically acceptable carrier, diluent or excipient in a form suitable for injection, or a form suitable for oral, rectal, vaginal, topical, nasal, transdermal or ocular administration. The compositions and substances of the aspects of the present technology may also comprise a further active component such as, for example, a vaccinating antigen (including recombinant antigens), an antigenic peptide sequence, or an immunoglobulin. Alternatively, the active component may be a macrophage stimulator, a polynucleotide molecule (e.g., encoding a vaccinating agent) or a recombinant viral vector.

The components of the vaccine and adjuvant compositions of the technology may be obtained through commercial sources, or may be prepared by one of ordinary skill in the art. The inulin particle formulations may be prepared by the processes disclosed in U.S. Provisional Patent Application No. 61/243,975 and international Patent Applications PCT/AU86/00311 (WO 87/02679), PCT/AU89/00349 (WO 90/01949) and PCT/AU2005/001328 (WO 2006/024100) or may be obtained commercially from Vaxine Pty Ltd, Adelaide, Australia. PAMP innate immune activators for use in the technology may be obtained commercially or made using methods well known in the art. For example, synthetic triacylated lipoprotein, Pam3CSK4 (0.25 μg/mouse), heat killed Listeria monocytogenes (2.5×10e7 cells/mouse), lipoarabinomannan from M. smegmatis (0.25 μg/mouse), LPS-PG ultrapure lipopolysaccharide from P. gingivalis (2.5 μg/mouse), standard lipoteichoic acids (LTA-SA) from S. aureus (2 μg/mouse), peptidoglycan from Staphylococcus aureus (PGN-SA) (2 μg/mouse), synthetic diacylated lipoprotein (0.25 μg/mouse), zymosan (1 mg/mouse), and CpG2006 (20 μg/mouse) as used in the current technology were all purchased from Invivogen, San Diego, USA. Synthetic CpG ODN synthesized with a native or modified phosphorothioate backbone was purchased from Geneworks, Australia and can be obtained from other commercial suppliers. MPLA may be purchased from Sigma, USA or Invivogen, San Diego, USA. Plasmid DNA may also be prepared using methods well known in the art, for example using the Quiagen procedure (Quiagen Inc, USA), followed by DNA purification using known methods. The inactivated or recombinant antigens used for immunization can be obtained through commercial chemical or protein suppliers such as Sigma, USA or may be prepared using methods well known in the art.

Biological activity of a vaccine may be assayed using standard laboratory techniques, e.g., by vaccinating a standard laboratory animal (e.g., a mouse or guinea pig) with a standard antigen (e.g., tetanus toxoid) using a test immunological formulation. After allowance of time for boosting the vaccination, and time for immunization to occur, the animal is bled or the spleen removed and the response to the vaccine measured. The response may be quantified by any measure accepted in the art for measuring immune responses, e.g., serum, saliva, vagina, stool antibody titer against the standard antigen (for measurement of humoral immunity) and T-cell proliferation, cytokine ELISPOT or cytokine ELISA assay (for measurement of T-cell immunity).

It will be apparent to one of ordinary skill in the art that the precise amounts of protein antigen and immunological composition needed to produce a given effect will vary with the particular compounds and antigens, and with the size, age, species, and condition of the subject to be treated. In certain embodiments, these amounts can be determined using methods known to those of ordinary skill in the art. In general, one or more vaccinations with the desired antigen are initially administered by intramuscular, subcutaneous or intradermal injection to prime the immune response. The vaccination is then “boosted” after a delay (usually from 1-12 months, for example, 6 months) using the immunological composition of the technology preferably by administering on one or more occasions the antigen combined with the immunological composition by parenteral injection for systemic immune boosting. Generally the antigen dose used for an adult human will be in the range of 0.001-0.1 mg and most commonly 0.001-0.1 mg, or 0.005-0.05 mg per dose.

In various embodiments, 0.1 to 5.0 mL or 0.1 to 1 mL of a vaccine is administered in the practice of the technology such as to a human subject.

The compositions and substances according to the aspects of the present technology is/are, in various embodiments, administered by intramuscular or intradermal injection, or other parenteral means, or by ballistic application to the epidermis. They may also be administered by intranasal application, inhalation, topically, intravenously, orally, or as implants, and even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249:1527-1533, 1990, which is incorporated herein by reference.

In certain embodiments, the immunological compositions are prepared and administered in dose units. Liquid dose units are vials or ampoules for injection or other parenteral administration. Solid dose units are tablets, capsules and suppositories. The administration of a given dose can be carried out both by single administration in the form of an individual dose unit or else several smaller dose units. Multiple administration of doses at specific intervals of weeks or months apart can be used for boosting antigen-specific immune responses.

The compositions and substances of the aspects of the present technology, or antigens useful in the technology, may be delivered in mixtures of more than two components. A mixture may comprise the immunological composition including one or more types of inulin particles (or other equivalent anti-inflammatory component) together with one or more PAMP innate immune activators and one or more antigens.

Immunogenic Compositions

In certain embodiments, disclosed herein is compositions of immunogens, wherein the immunogens comprise a region A coupled to a region B. Region A is an active component of vaccine that is responsible for induction of therapeutic antibodies. Region B is a helper component that is responsible for induction of cellular immune responses that help B cells to produce antibodies.

In certain embodiments, region A comprises (i) at least one Amyloid-β (Aβ) B cell epitope or (ii) at least one Tau B cell epitope or (iii) at least one α-synuclein (α-syn) B cell epitope or (iv) at least one Amyloid-β (Aβ) B cell epitope and at least one Tau B cell epitope or (v) at least one Amyloid-β (Aβ) B cell epitope and at least one α-synuclein (α-syn) B cell epitope or (vi) at least one Tau B cell epitope and at least one α-synuclein (α-syn) B cell epitope or (vii) at least one Amyloid-β (Aβ) B cell epitope and at least one Tau B cell epitope and at least one α-synuclein (α-syn) B cell epitope. In certain embodiments, when multiple epitopes are present in Region A, the epitopes may comprise the same epitopic sequence (e.g., multiple copies of Aβ) or different epitopic sequences (e.g., Aβ and tau₂₋₁₈). When Region A has different epitopes, the order of the epitopes may be arbitrary or optimized based on in vitro or in vivo tests.

In certain embodiments, region B comprises at least one foreign T helper cell (Th) epitope. In certain embodiments, when multiple T cell epitopes are present in Region B, the epitopes may comprise the same epitopic sequence (e.g., multiple copies of PADRE) or different epitopic sequences (e.g., PADRE and tetanus toxin p23). When Region B has different epitopes, the order of the epitopes may be arbitrary or optimized based on in vitro or in vivo tests.

In certain embodiments, when two or more immunogens are present in a composition, the immunogens are distinct (i.e., not identical) in region A or region B or both. For the purposes of this disclosure, if two regions contain the same number of epitopes and the same sequence of epitopes, if the arrangement varies then the regions, and hence the immunogens, are distinct. That is, a region comprising epitope 1 and epitope 2 in the order 1-2 is distinct from the order 2-1.

In another aspect, the composition comprises nucleic acid molecules that encode immunogens that comprise a region A coupled to a region B. In certain embodiments, region A comprises (i) at least one Amyloid-β (Aβ) B cell epitope or (ii) at least one Tau B cell epitope or (iii) at least one α-synuclein (α-syn) B cell epitope or (iv) at least one Amyloid-β (Aβ) B cell epitope and at least one Tau B cell epitope or (v) at least one Amyloid-β (Aβ) B cell epitope and at least one α-synuclein (α-syn) B cell epitope or (vi) at least one Tau B cell epitope and at least one α-synuclein (α-syn) B cell epitope or at least one Amyloid-β (Aβ) B cell epitope and at least one Tau B cell epitope and at least one α-synuclein (α-syn) B cell epitope. Region B comprises at least one foreign T helper cell (Th) epitope. When multiple epitopes are present in Region A, the epitopes may comprise the same epitopic sequence (e.g., multiple copies of Aβ₁₋₁₁) or different epitopic sequences (e.g., Aβ₁₋₁₁ and tau₂₋₁₈). When Region A has different epitopes, the order of the epitopes may be arbitrary or optimized based on in vitro or in vivo tests.

In certain embodiments, region B comprises at least one foreign T helper cell (Th) epitope. When multiple T cell epitopes are present in Region B, the epitopes may comprise the same epitopic sequence (e.g., multiple copies of PADRE) or different epitopic sequences (e.g., PADRE and tetanus toxin p23). When Region B has different epitopes, the order of the epitopes may be arbitrary or optimized based on in vitro or in vivo tests.

In certain embodiments, when two or more immunogens are encoded, the immunogens are distinct (i.e., not identical) in region A or region B or both. For the purposes of this disclosure, if two regions contain the same number of epitopes and the same sequence of epitopes, if the arrangement varies then the regions, and hence the immunogens, are distinct. That is, a region comprising epitope 1 and epitope 2 in the order 1-2 is distinct from the order 2-1. Multiple immunogens may be encoded by a single nucleic acid molecule or a single immunogen may be encoded by a single nucleic acid molecule. In some embodiments, at least two immunogens are encoded on a single nucleic acid molecule. In other embodiments, each of the immunogens is encoded by separate nucleic acid molecules. In yet other embodiments, more than one immunogen is encoded by a single nucleic acid molecule and at least one other immunogen is encoded by a separate nucleic acid molecule.

In various embodiments, the at least one epitope in Region A and Region B can be about 1 to about 18, or about 1 to about 15, or about 1 to about 12, or about 1 to about 9, or about 1 to about 6, or about 1 to about 3, or 1, or 2, or 3, or 4, or 5, or 6, or 7, or 8, or 9, or 10, or 11, or 12 or 13 or 14 or 15 or 16 or 17 or 18 amino acids. When there is more than one epitope, the epitopes may all be different sequences, or some of them may be different sequences.

In some embodiments, the at least one Th epitope of region B is capable of being recognized by one or more antigen-experienced T helper cell populations of a subject. The composition is normally capable of activating a humoral immune response in a subject. In some embodiments, the humoral immune response comprises one or more antibodies specific to pathological forms of Aβ, or Tau, or α-syn proteins.

1. Structure of B Cell Epitopes

A B cell epitope is a peptide comprising a sequence that can stimulate production of antibodies by B cells that bind to the epitope or protein containing the epitope. Moreover, the B cell epitope within the context of this disclosure preferably does not stimulate a T cell response. In certain embodiments, the B cell epitopes herein may comprise additional sequence, such as amino acids that flank the epitope in the native protein. For example if the minimal sequence of a B cell epitope is amino acids 5-11, a B cell epitope herein may comprise additional amino acids such as residues 3-15. Typical B cell epitopes are from about 5 to about 30 amino acids long. In some embodiments, the sequence of the at least one Aβ B cell epitope is located within SEQ ID NO: 1, wherein the epitope is less than 42 amino acids long. In some embodiments, the epitope is 15 amino acids in length and in other embodiments, it is less than 15 amino acids in length, i.e., 14, 13, 12, 11, 10, 9, 8, 7, 6, 5, or 4 amino acids. In some embodiments, the epitope comprises the sequence DAEFRH (SEQ ID NO: 7).

In some embodiments, the sequence of at least one Tau B cell epitope is located within SEQ ID NO: 2. Typically, the epitope will be from about 5 to about 30 amino acids long. In some embodiments, the epitope is 12 amino acids in length and in other embodiments, it is less than 12 amino acids in length, i.e., 11, 10, 9, 8, 7, 6, or 5 amino acids. In some embodiments, the epitope comprises the sequence AKAKTDHGAEIVYKSPWSGDTSPRHLSNVSSTGSID (SEQ ID NO: 8). In other embodiments, the epitope comprises the sequence RSGYSSPGSPGTPGSRSR (SEQ ID NO: 9), or the sequence NATRIPAKTPPAPKTPPSSGEPPKSGDRSGYSSPGS (SEQ ID NO: 10), or the sequence GEPPKSGDRSGYSSPGSPGTPGSRSRTPSLPTPPTREPKK (SEQ ID NO: 11), or the sequence KKVAWRTPPKSPSS (SEQ ID NO: 12), or the sequence AEPRQEFEVMEDHAGTY (SEQ ID NO: 13). In certain embodiments, the epitope comprises at least 5 contiguous amino acids of SEQ ID NOs: 8-13.

In some embodiments, the sequence of at least one α-syn B cell epitope of region A is located within SEQ ID NO: 3. The epitope will often be about 5 to 50 amino acids long. In some embodiments, the epitope is about 50 amino acids long; in other embodiments, the epitope is less than about 50 amino acids, in still other embodiments, the epitope is less than about 30 amino acids, or less than about 20 amino acids, or less than about 15 amino acids, or less than about 12 amino acids. In certain embodiments, the fragment comprises the sequence:

| | SEQ ID NO: |
|---|---|
| KTKEGVLYVGSKTKEGVVHGVATVAEKTKEQV | 14 |
| TNVGGAVVTGVTAVAQK | |
| AGSIAAATGFVKKDQ | 15 |
| QEGILEDMPVDPDNEAYE | 16 |
| EMPSEEGYQDYEPEA | 17 |
| KAKEG | 18 |
| GKTKEGVLYVGSKTKEGVVH | 42 |
| EGVVHGVATVAEKTKEQVTNVGGA | 43 |
| EQVTNVGGAVVTGVTAVAQK | 44 |

In certain embodiments, the epitope comprises at least 5 contiguous amino acids of SEQ ID NOs: 14-18 and 42-44.

In some embodiments, region A comprises a plurality of B cell epitopes. In certain embodiments, region A comprises 1, 2, or 3 B cell epitopes. In other embodiments, region A comprises as many as 18 epitopes, e.g., 1, 2, 3, 4, 5, 6, 7, 8, 9, 10, 11, 12, 13, 14, 15, 16, 17 or 18. The plurality of epitopes can have identical sequences or different sequences. Furthermore, the plurality of epitopes can be all one type—i.e., all having a tau sequence, all having an Aβ sequence, or all having an α-syn sequence. In some embodiments, the plurality of epitopes are from a combination of tau, Aβ, and α-syn. In some embodiments, Region A comprises three Aβ, three tau, and three α-synuclein epitopes. In particular embodiments, the Aβ epitopes comprise residues 1-11, the tau epitopes comprise residues 2-13, and α-synuclein epitopes comprise residues 36-39. In other embodiments, Region comprises three Aβ and three tau epitopes. In particular embodiments, the Aβ epitopes comprise residues 1-11 and the tau epitopes comprise residues 2-13. When region A comprises a plurality of B cell epitopes (or encodes a plurality of B cell epitopes), the epitopes are typically present in a tandem array with linkers between them. The linkers may be of any length and sequence, although short sequences of flexible residues like glycine and serine that allow adjacent protein domains to move freely relative to one another are typically used. Longer linkers may be used in order to ensure that two adjacent domains do not sterically interfere with one another. An exemplary linker sequence is GS (glycine-serine).

In some embodiments, an Aβ B cell epitope may be encoded by a sub-sequence shown in SEQ ID NO: 4 or a nucleic acid sequence that encodes the amino acids. Similarly, a Tau B cell epitope may be encoded by the sequence or sub-sequence shown in SEQ ID NO: 5, or by a nucleic acid sequence that encodes the same amino acids, or an α-syn B cell epitope may be encoded by the sequence or a sub-sequence shown in SEQ ID NO: 6, or by a nucleic acid sequence that encodes the same amino acids.

B cell epitopes of Aβ, tau and α-syn may be identified in a variety of ways, including but not limited to computer program analysis, peptide arrays, phage display libraries, direct binding assays, etc. Computer programs, as well as other tests are commercially or freely available, can be used to predict or directly show B cell epitopes. Candidate sequences can be synthesized and coupled to a carrier protein that is used to immunize an animal, e.g., a mouse. Sera may then be tested by ELISA or other known method for the presence of antibodies to the candidate. In addition, the epitopes may be tested by any method known in the art or described herein for stimulation of T cells.

In certain embodiments, suitable epitopes do not stimulate T cells. Some peptides of Aβ are known to act as a T cell epitope. These include the sequences, QKLVFFAEDVGSNKGAIIGLMVGGWIA (SEQ ID NO: 19), VFFAEDVGSNKGAII (SEQ ID NO: 20), QKLVFFAEDVGSNKGAIIGL (SEQ ID NO: 21), LVFFAEDVGSNKGA (SEQ ID NO: 22), QKLVFFAEDVGSNKG (SEQ ID NO: 23), and GSNKGAIIGLMVGGVVIA (SEQ ID NO: 24). Other B cell epitope candidates can be assayed for T cell epitope function using one of the assays described herein or known in the art, such as [3H]thymidine incorporation upon stimulation, MHC-binding assays, intracellular staining, ELISPOT, flow cytometry of CFSE-stained proliferating cells, MTA proliferation assay, that can be used to identify epitope sequences that elicit helper T cell proliferation and thus potentially cause a helper T cell immune responses in subject receiving the composition.

2. T Cell Epitopes (MultiTEP Platform for Vaccines)

In certain embodiments, the T cell epitopes of the immunogens are “foreign”, that is, they are peptide sequences or encode peptide sequences that are not found in the mammals and in the subject to receive the composition. A foreign T cell epitope can be derived from a non-self non-mammalian protein or be an artificial sequence. PADRE is an example of an artificial sequence that serves as a T cell epitope. A “promiscuous T cell epitope” means a peptide sequence that can be recognized by many MHC-II (e.g., human DR) molecules of the immune system and induce changes in immune cells of these individuals such as, but not limited to production of cytokine and chemokines. The T cells specific to these epitopes help B cells, such as B cells specific to amyloid or tau or α-synuclein to produce antibodies to these proteins. It is desirable that antibody produced be detectable and moreover produced at therapeutically relevant titers against pathological forms of these proteins in the sera of vaccinated subjects.

As discussed herein, in certain embodiments the T cell epitope is foreign to the subject receiving the composition. In some embodiments, the at least one Th epitope of one or more of the immunogens is from 12 to 22 amino acids in length. Region B may comprise a plurality of Th epitopes, either all having the same sequence or encoding the same sequence, or a mixture of different Th epitopes. In some embodiments, region B comprises from 1 to 20 epitopes, in other embodiments, region B comprises at least 2 epitopes, in yet other embodiments region B comprises from 2 to about 20 epitopes. Exemplary B regions are illustrated in the Figures and Examples. When region B comprises a plurality of T cell epitopes (or encodes a plurality of T cell epitopes), the epitopes are typically present in a tandem array with linkers between them. The linkers may be of any length and sequence, although short sequences of small amino acids will usually be used. An exemplary linker sequence is GS (glycine-serine). Collectively the string of Th epitopes is called MultiTEP platform:

(SEQ ID NO: 45)
AKFVAAWTLKAAAGSVSIDKFRIFCKANPKGSLKFIIKRYTPNNEIDSGS
IREDNNITLKLDRCNNGSFNNFTVSFWLRVPKVSASHLEGSQYIKANSKF
IGITEGSPHHTALRQAILCWGELMTLAGSFFLLTRILTIPQSLDGSYSGP
LKAEIAQRLEDVGSNYSLDKIIVDYNLQSKITLPGSLINSTKIYSYFPSV
ISKVNQGSLEYIPEITLPVIAALSIAES*.

There are many suitable T cell epitopes. Epitopes can be identified by a variety of well-known techniques, including various T cell proliferation assays as well as using computer algorithms on protein sequences and MHC-binding assays, or chosen from myriad databases, such as MHCBN (hosted at EMBL-EBI), SYFPEITHI (hosted by the Institute for Cell Biology, BMI-Heidelberg and found at (www.syfpeithi.de), IEDB (Vita R, et al. Nucleic Acids Res. 2010 38(Database issue):D854-62. Epub 2009 Nov. 11, and found at www.iedb.org), and SEDB (hosted at Pondicherry University, India, and found at sedb.bicpu.edu. in). T cell epitopes presented by MHC class I molecules are typically peptides between 8 and 11 amino acids in length, whereas MHC class II molecules present longer peptides, typically 13-17 amino acids in length.

In some embodiments, the at least one Th epitope (peptide binding to MHC class II and activating Th cell) is one or more of a Tetanus toxin epitope, a diphtheria toxin epitope, a Hepatitis B surface antigen epitope, an influenza virus hemagglutinin epitope, an influenza virus matrix protein epitope, one or more synthetic promiscuous epitopes, or mixtures thereof. For example, suitable Th epitopes include a P23TT Tetanus Toxin epitope comprising the sequence VSIDKFRIFCKANPK (SEQ ID NO: 25), a P32TT Tetanus Toxin epitope comprising the sequence LKFIIKRYTPNNEIDS (SEQ ID NO: 26), a P21TT Tetanus Toxin epitope comprising the sequence IREDNNTLKLDRCNN (SEQ ID NO: 27), a P30TT Tetanus Toxin epitope comprising the sequence FNNFTVSFWLRVPKVSASHLE (SEQ ID NO: 28), a P2TT Tetanus Toxin epitope comprising the sequence QYIKANSKFIGITE (SEQ ID NO: 29), a Tetanus Toxin epitope comprising the sequence LEYIPEITLPVIAALSIAES (SEQ ID NO: 30), a Tetanus Toxin epitope comprising the sequence LINSTKIYSYFPSVISKVNQ (SEQ ID NO: 31), a Tetanus Toxin epitope comprising the sequence NYSLDKIIVDYNLQSKITLP (SEQ ID NO: 32), a HBV nuclear capsid epitope comprising the sequence PHHTALRQAILCWGELMTLA (SEQ ID NO: 33), a HBV surface antigen epitope comprising the sequence FFLLTRILTIPQSLD (SEQ ID NO: 34), a MT Influenza matrix epitope comprising the sequence YSGPLKAEIAQRLEDV (SEQ ID NO: 35), a PADRE epitope comprising the sequence AKFVAAWTLKAAA (SEQ ID NO: 36) and a PADRE epitope comprising the sequence aK-Cha-VAAWTLKAAa, (SEQ ID NO: 40) where “a” is D alanine and Cha is L-cyclohexylalanine. In some embodiments, the MultiTEP platform is encoded by a nucleic acid molecule.

Construction/Preparation of Immunogens

When the immunogens are to be delivered as a DNA composition, the composition will typically be an expression vector. In some embodiments, the vector is capable of autonomous replication. In other embodiments, the vector is a viral vector or a bacterial vector. The vector can alternatively be a plasmid, a phage, a cosmid, a mini-chromosome, or a virus. The sequence encoding an immunogen will be operatively linked to a promoter that is active in host cells. There will typically also be a polyA signal sequence, one or more introns, and optionally other control sequences, such as an enhancer. The promoter can be a constitutive promoter, an inducible promoter, or cell-type specific promoter. Such promoters are well known in the art.

The nucleic acid constructs may also be used to produce a polypeptide immunogen. In this case, the construct(s) are transfected or introduced into host cells in vitro and protein is isolated. Protein may be purified by any of a variety of techniques, including precipitation, affinity chromatography, and HPLC. Suitable host cells include bacteria, yeast cells, insect cells, and vertebrate cells. The choice of a host cell depends at least in part on the backbone of the construct. Affinity tags, such as FLAG and hexa-His may be added to the immunogen to facilitate isolation purification.

Also disclosed herein is a method of making a composition disclosed herein, comprising: obtaining sequence data representing the sequence of the composition; and synthesizing the composition. Resulting proteins may be used without further purification or purified by any of a variety of protein purification methods, including HPLC and affinity chromatography.

Coupling of Regions

In certain embodiments, the A and B regions of the at least two immunogens are coupled. When two or more immunogens are used, the two or more immunogens may also be coupled. Coupling may be through a chemical linkage or peptide linkage (e.g., a fusion protein) or electrostatic interaction (e.g., van der Waals forces) or other type of coupling.

When the linkage is peptidic, the C-terminus of region A may be linked to the N-terminus of region B or vice versa. Alternatively, C-terminus of one B region may be coupled to N-terminus of A region and N-terminus of another B region may be coupled to the C-terminus of the same A region. Moreover, region A may be coupled to region B via a linker domain. Linker domains can be any length, as long as several hundred amino acids, but more typically will be 2-30 amino acids or equivalent length. Linkers are often composed of flexible residues like glycine and serine that allows adjacent protein domains to move freely relative to one another. Longer linkers are used in order to ensure that two adjacent domains do not sterically interfere with one another. Some exemplary linkers include the sequences GS, GSGSG (SEQ ID NO: 37), or YNGK (SEQ ID NO: 38). In some embodiments, one or more of the linkers comprise a helix-forming peptide, such as A(EAAAK)nA (SEQ ID NO: 39), where n is 2, 3, 4, or 5. Alternatively, two immunogens may be synthesized as a multiple antigen peptide (MAP) coupled through 4 or 8 lysine branch.

Chemical cross-linking is an alternative to coupling regions A and B or the at least two immunogens. Linkers and cross-linkers are well-known and commercially available from e.g., Aldrich Co. and ThermoScientific.

Formulations and Delivery

In certain embodiments, the immunogen or immunogens is typically formulated with a pharmaceutically-acceptable excipient. Excipients include normal saline, other salts, buffers, carriers, buffers, stabilizers, binders, preservatives such as thimerosal, surfactants, etc. and the like. Such materials are preferably non-toxic and minimally interfere (or not interfere at all) with the efficacy of the immunogen. The precise nature of the excipient or other material can depend on the route of administration, e.g. oral, intravenous, cutaneous or subcutaneous, nasal, intramuscular, intraperitoneal routes. In some embodiments, compositions are formulated in nano particles and liposomes.

In some embodiments, the composition further comprises an adjuvant. Suitable adjuvants include aluminum salts, such as aluminum hydroxide, aluminum phosphate and aluminum sulfates, saponin adjuvants (e.g., QS-21), 3 De-O-acylated monophosphoryl lipid A (MPL), Montanide, CpG adjuvant, MF59, Inulin-based adjuvant, nanoparticle and liposomal adjuvants. They may be formulated as oil in water emulsions, such as with squalene, or in combination with immune stimulants, such as MPL. Adjuvants can be administered as a component of a therapeutic composition with an active agent or can be administered separately, before, concurrently with, or after administration of the immunogenic therapeutic agent. Other adjuvants include chemokines (e.g., MDC) and cytokines, such as interleukins (IL-1, IL-2, IL4, and IL-12), macrophage colony stimulating factor (M-CSF), tumor necrosis factor (TNF), etc.

The compositions herein can be administered by any suitable delivery route, such as intradermal, mucosal (e.g., intranasal, oral), intramuscular, subcutaneous, sublingual, rectal, vaginal. The intramuscular (i.m.) route is one such suitable route for the composition. Suitable i.m. delivery devices include a needle and syringe, a needle-free injection device (for example Biojector, Bioject, Oreg. USA), or a pen-injector device, such as those used in self-injections at home to deliver insulin or epinephrine. Intradermal (i.d.) and subcutaneous (s.c.) delivery are other suitable routes. Suitable devices include a syringe and needle, syringe with a short needle, and jet injection devices, etc. The composition may be administered by a mucosal route, e.g., intranasally. Many intranasal delivery devices are available and known in the art. Spray devices are one such device. Oral administration can be as simple as providing a solution for the subject to swallow.

In certain embodiments, the composition may be administered at a single site or at multiple sites. If at multiple sites, the route of administration may be the same at each site, e.g., injection in different muscles, or may be different, e.g., injection in a muscle and intranasal spray. Furthermore, it may be administered i.m., s.c, i.d., etc. at a single time point or multiple time points. Generally if administered at multiple time points, the time between doses has been determined to improve the immune response.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

Compositions comprising nucleic acid may be delivered intramuscularly, intradermally by e.g., electroporation device, intradermally by e.g., gene gun or biojector, by patches or any other delivery system.

Whether it is a polypeptide or nucleic acid that is to be given to an individual, the amount administered is preferably a “therapeutically effective amount” or “prophylactically effective amount”. As used herein, “therapeutically effective amount” refers to an amount that is effective to ameliorate a symptom of a disease. A therapeutically effective amount can be a “prophylactically effective amount” as prophylaxis is also therapy. The term “ameliorating” or “ameliorate” is used herein to refer to any therapeutically beneficial result in the treatment of a disease state or symptom of a disease state, such as lessening the severity of disease or symptoms, slowing or halting disease progression, causing a remission, effecting a cure, delaying onset, or effecting fewer or less severe symptoms of a disease when it occurs.

The actual amount administered, and rate and time-course of administration, will depend on the nature and severity of protein aggregation disease being treated. Prescription of treatment, e.g., decisions on dosage is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

The compositions disclosed herein can be administered as sole treatment or provided in combination with other treatments (medical and non-medical), either simultaneously or sequentially dependent upon the condition to be treated.

Also disclosed herein are, in certain embodiments, methods of inducing an immune response in a subject in need thereof, comprising administering a sufficient amount of a composition disclosed herein. The term “sufficient amount” is used herein to mean an amount sufficient to produce a desired effect, e.g., an amount sufficient to modulate protein aggregation in a cell or raise an immune response. The composition may comprise one or more of the immunogens. Additives, such as adjuvants, are optional. Usually, the composition administered is a pharmaceutical composition comprising one or more immunogens. In some aspects, the subject has been diagnosed with Alzheimer's disease or one or more conditions associated with abnormal amyloid deposits, Tau deposits, or α-syn deposits or will be at risk of getting Alzheimer's disease or one or more conditions associated with abnormal amyloid deposits, Tau deposits, or α-syn deposits. An immune response is generated by administration of one of the compositions disclosed herein. An immune response can be verified by assay of T cell stimulation or production of antibodies to the B cell epitope(s). Immunoassays for antibody production are well known and include ELISA, immunoprecipitation, dot blot, flow cytometry, immunostaining and the like. T cell stimulation assays are also well-known and include proliferation assays, cytokine production assays, detection of activation markers by flow cytometry and the like.

Also disclosed herein are, in certain embodiments, methods of treating or ameliorating a condition associated with deposits of amyloid, tau, or α-syn, comprising administering to a subject in need thereof an effective amount of a composition disclosed herein. In general, amelioration can be determined when the total amount of amyloid, Tau protein, or α-syn deposits is decreased post-administration, relative to a control. Other biochemical tests or neuropathology tests can be used, such as determination of ratio of phosphorylated and unphosphorylated tau to Aβ₄₂ peptide in CSF, PET-scan with dyes (e.g., Pittsburgh compound B or ¹⁸F-FDDNP) binding to β-Amyloid plaques in brain, less aggregation of the proteins, prevention or slowing of the development of dystrophic neurites, and reduced astrogliosis. Other methods of determining amelioration include cognitive function assays. Amelioration may be manifest as a delay of onset of cognitive dysfunction or memory impairment, a significantly slower rate of decline of cognitive functions and an improvement in the activities of daily living.

Methods of treatment of Aβ, Tau, and α-syn related diseases are also encompassed. β-Amyloid (Aβ), tau, and α-synuclein (α-syn) are the primary components of amyloid plaques (Aβ-plaques), neurofibrillary tangles (NFT), and Lewy bodies (LBs), respectively. Many neurodegenerative disorders are characterized by the presence of one or more of these lesions. For example, Alzheimer's disease (AD) is characterized by the accumulation of Aβ plaques and neurofibrillary tangles. A subtype of AD also displays αsyn-bearing LBs.

Said methods of the invention include administering a therapeutically effective amount of a composition and/or compositions disclosed herein.

In order that the nature of the present technology may be more clearly understood, preferred forms thereof will now be described with reference to the following non-limiting examples. The entire contents of all of the references (including literature references, issued patents, published patent applications, and co-pending patent applications) cited throughout this application are hereby expressly incorporated by reference.

### EXAMPLES

The use of inulin particles in vaccines for neurodegenerative diseases, either alone or in combination with other immune activators, was evaluated. Recombinant hepatitis B virus surface antigen (HBsAg) and influenza virus antigen were used as exemplary model systems in some examples set forth below. Alzheimer's disease (AD) epitope vaccines based on amyloid-β, tau or combination of amyloid-β and tau, as well as Parkinson disease (PD) epitope vaccine based on α-syn, were also used as exemplary model systems in the examples set forth below.

### Example 1

Preparation of Adjuvant Compositions

Inulin particle formulations referred to in the following examples were prepared as described below.

Gammulin (Gamma inulin; gIN), Algammulin (AG) and Phosgammulin (PG): Gamma inulin (gIN) and Algammulin were prepared as previously described in PCT/AU86/00311 (WO 87/02679) titled “Immunotherapeutic treatment”, and PCT/AU89/00349 (WO 90/01949) titled “Gamma inulin compositions”, which are hereby expressly incorporated by reference. To produce Phosgammulin (PG), a 5% suspension of gIN in water was first dissolved by heating at 80-85° C. then mixed with a fine suspension of aluminum phosphate gel (Adju-Phos™ Aluminum Phosphate Gel Adjuvant 0.44%, BrenntagBiosector, Frederickssund, Denmark) in a proportion to give an inulin:Adju-Phos™ weight/weight ratio of between 2 and 200. The suspension was then crystallized at 5° C., then converted to the gamma form (1 hour at) 45° to yield Phosgammulin hybrid particles, and washed and formulated as appropriate.

Deltin (Delta inulin; dIN): Deltin (dIN) was prepared from gIN as previously described in WO 2006/024100, which is hereby expressly incorporated by reference. Briefly, a standard formulation of gIN in water (200 mL at 50 mg/mL) was incubated for 1 hour in a water bath at 55° C., which was then raised to 60° C. for 30 min. The particles were then centrifuged, resuspended in water at 55° C., re-incubated at 55° C. and washed again in the same manner, before being finally resuspended in 50 mL cold water. This treatment is sufficient to remove much of the inulin present in the alpha and gamma forms. A sample of the dIN-enriched suspension dissolved completely at 80-85° C. The refractive index indicated a concentration of 48 mg/mL. The Deltin suspension used in these experiments had a concentration of 5% weight/volume of water.

Phosdeltin (dIN/aluminum phosphate preparation (PD)): To produce Phosdeltin (PD), a 5% suspension of Deltin as described above was first dissolved in water by heating at 80-85° C. then mixed with a fine suspension of aluminum phosphate gel (Adju-Phos™ Aluminum Phosphate Gel Adjuvant 0.44%, BrenntagBiosector, Frederickssund, Denmark) in a proportion to give an dIN:Adju-Phos™ weight/weight ratio of between 2 and 200. The suspension was then crystallized at 5° C., then converted to gIN (1 hr at 45°) then to dIN (1 hr at 55° C.) to yield Phosdeltin hybrid particles, and washed and formulated as appropriate.

Aldeltin (dIN/aluminum hydroxide preparation): To produce Aldeltin (AD), the same procedure was followed as above for Phosdeltin except that a fine suspension of aluminum hydroxide gel (Alhydrogel™ Aluminum Hydroxide Gel Adjuvant, Al (calc) 3.0%, BrenntagBiosector, Frederickssund, Denmark) was used instead of aluminum phosphate gel. In brief, a 5% suspension of Deltinin water as described above was first dissolved by heating at 80-85° C. then mixed with a fine suspension of Alhydrogel™ in a proportion to give an dIN:Alhydrogel™ weight/weight ratio of between 2 and 200. The suspension was then crystallized at 5° C., then converted to gIN (1 hr at 45°) and then to dIN (1 hr at 55° C.) to yield Aldeltin hybrid particles, and washed and formulated as appropriate.

Epsilin (eIN): Epsilin was prepared from dIN as described in PCT/AU2010/001221 titled “A novel epsilon polymorphic form of inulin and compositions comprising same”. In brief, EI was prepared by heating a concentrated suspension of greater than 50 mg/mL of dIN at 60° C. for one hour.

Phosepsilin (PE): To produce Phosepsilin (PE), a 5% suspension of eINin water as described above was first dissolved by heating at 80-85° C. then mixed with a fine suspension of aluminum phosphate gel (Adju-Phos™ Aluminum Phosphate Gel Adjuvant 0.44%, BrenntagBiosector, Frederickssund, Denmark) in a proportion to give an eIN:Adju-Phos™ weight/weight ratio of between 2 and 200. The suspension was then crystallized at 5° C., then converted to gIN (1 hr at 45°) then to the dIN form (1 hr at 55° C.) then to the eIN form to yield Phosepsilin hybrid particles, and washed and formulated as appropriate. Alepsilin (AE) was similarly made by substituting Alhydrogel™ instead of Adju-Phos™ in the above process for making Phosepsilin.

PGmix, PDmix and PEmix: Phosdeltin (dIN/aluminum phosphate) and dIN formulations, as described above, were admixed to form a mixed suspension of particles some containing pure inulin and others containing inulin with aluminum phosphate (PDmix). For the experiments described herein, the PDmix Phosdeltin:Deltin combination adjuvant was prepared in various ratios ranging from 1:1 to 1:36 weight for weight of inulin content of inulin-alum amalgam particles and inulin particles, respectively, hereinafter referred to as PDmix1:1 to PDmix1:36) This enabled the amount of aluminum phosphate containing particles to be varied relative to the number of non-aluminum salt containing dIN particles. PGmix and PEmix were prepared in the same manner. The ratio of Phosdeltin to Deltin particles is expressed as x:y PD:D). This means that x amount of PD based on inulin content was mixed with y amount of dIN based on inulin content to form PDmixx:y.

AGmix, ADmix and AEmix: To make AD mix, Aldeltin and Deltin formulations, as described above, were admixed to form a mixed suspension. For the experiments described herein, the Aldeltin:Deltin combination adjuvant was prepared in various ratios ranging from 1:1 to 1:36 weight for weight of inulin content, thereby enabling the amount of Alhydrogel containing particles to be varied relative to the number of non-aluminum containing dIN particles. AG and AE were prepared in the same manner.

PAMP Innate Immune Activators: PAMP innate immune activators including synthetic triacylated lipoprotein (Pam3CSK4) (0.25 μg/mouse), heat-killed Listeria monocytogenes (2.5×10e7 cells/mouse), lipoarabinomannan from M. smegmatis (0.25 μg/mouse), LPS-PG ultrapure lipopolysaccharide from P. gingivalis (2.5 μg/mouse), standard lipoteichoic acids from S. aureus (LTA-SA) (2 μg/mouse), peptidoglycan from Staphylococcus aureus (PGN-SA) (2 μg/mouse), synthetic diacylated lipoprotein (0.25 μg/mouse), zymosan (1 mg/mouse), CpG2006 (20 μg/mouse) and monophosphoryl lipid A were all purchased from Invivogen, San Diego, USA and used per the manufacturer's instructions. In addition, synthetic oligodeoxynucleotides (e.g., ODN1826 of the sequence TCCATGACGTTCCTGACGTT synthesized with a phosphorothioate backbone) were purchased from Geneworks, Australia. PAMP innate immune activators were dissolved according to the manufacturer's instructions and diluted into normal saline solution prior to use.

Formulation of Inulin Particles with PAMP Innate Immune Activators: Aqueous suspensions of gIN, dIN, eIN, AG, AD, AE, PG, PD, PE, PG mix, PDmix, PEmix, AGmix, ADmix or AEmix (collectively referred to as “inulin particles”), were prepared as described above. Individual TLR agonists and other PAMP innate immune activators as detailed above were pipetted into the relevant inulin particle suspension to give the desired final concentration. In the same manner, solutions of vaccine antigens, for example, influenza haemagglutinin or HBsAg, were simply pipetted into the relevant immunological formulation to give the desired final vaccine concentration. The mixture of antigen, PAMP innate immune activator and inulin particles was then immediately prior to immunization drawn up into a syringe ready for injection.

Mouse Immunizations: BALB/c mice at various ages and in group sizes of 5-10 mice per group were immunized intramuscularly in the hind-limb with 50 μl of vaccine in normal saline vehicle. Injections were carried out with a 0.3 mL insulin syringe that has a fused 29G needle (Becton Dickenson, Franklin Lakes, N.J.).

Evaluation of Humoral Response to Antigens: Heparinized blood was collected by retrobulbar puncture of lightly anaesthetized mice as described elsewhere (Michel et al., 1995). Plasma was recovered by centrifugation (7 min at 13,000 rpm). Antigen-specific antibodies in plasma were detected and quantified by an ELISA assay using a standard protocol. Dilutions of plasma were first added to 96-well microtiter plates coated with antigen overnight at room temperature (RT). The bound antibodies were then detected by incubation for 1 hour at 37 C with anti-mouse IgG, IgM, IgG1 or IgG2a conjugated to horse radish peroxidase (HRP) (1:2000 in PBS-Tween, 10% FCS; 100 μl/well), followed by incubation with TMB solution (100 μl/well, Sigma, St. Louis, Mo.) for 30 minutes at RT. The reaction was stopped by the addition of 1M sulfuric acid and absorbance read with an ELISA plate reader.

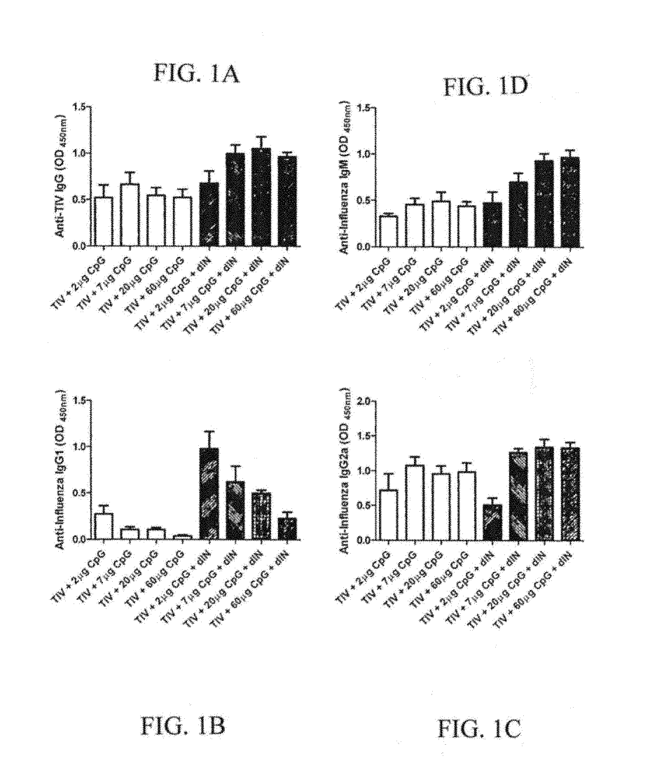

To determine whether there was a favorable dose-response relationship between a TLR9 agonist (CpG2006 ODN) and an inulin particle formulation (PDmix), female Balb/c mice at 6-8 weeks of age (n=5-8 per group) were immunized intramuscularly twice 14 days apart, with 50 ul of a commercial human trivalent inactivated influenza vaccine (TIV) (Fluvax® 2007) at 100 ng of haemagglutinin per dose, combined with either 2, 7, 20 or 60 μg of CpG2006 alone or mixed with 1 mg PDmix(1:5). Mice were bled 42 days after the second immunization and anti-influenza antibodies measured by ELISA (FIGS. 1A-1D). Increasing doses of CpG from 2 to 60 ug suppressed the anti-influenza IgG1 response at the same time as enhancing the anti-influenza IgG2a response. However, due to this suppression of IgG1 by the CpG, the overall anti-influenza total IgG response with CpG even at the highest CpG 60 μg dose was not significantly different to that achieved with TIV administered without adjuvant. However, the mice that received CpG2006 with PDmix inulin particles showed a synergistic enhancement of the anti-influenza IgG1 response particularly at the CpG 2 and 7 μg doses, which was in stark contrast to the inhibition of the anti-influenza IgG1 response seen with the same doses of CpG when given alone without inulin particles. The enhancement of total IgG with the combination confirms that inulin particles provide dose-sparing effects for a PAMP innate immune activator such that the benefits of the PAMP innate immune activator on the immune response are obtained at a lower dose when it is administered together with inulin particles. At the same time, the benefits of CpG in terms of enhancing the IgG2a response was retained or even enhanced in the presence of the inulin particles. The anti-influenza total IgG response was greatest in the group that received TIV plus PDmix inulin particles with the PAMP, CpG 60 μg. Similarly, the anti-influenza IgM response was also enhanced to the greatest degree in the CpG and PDmix combination groups.

### Example 2

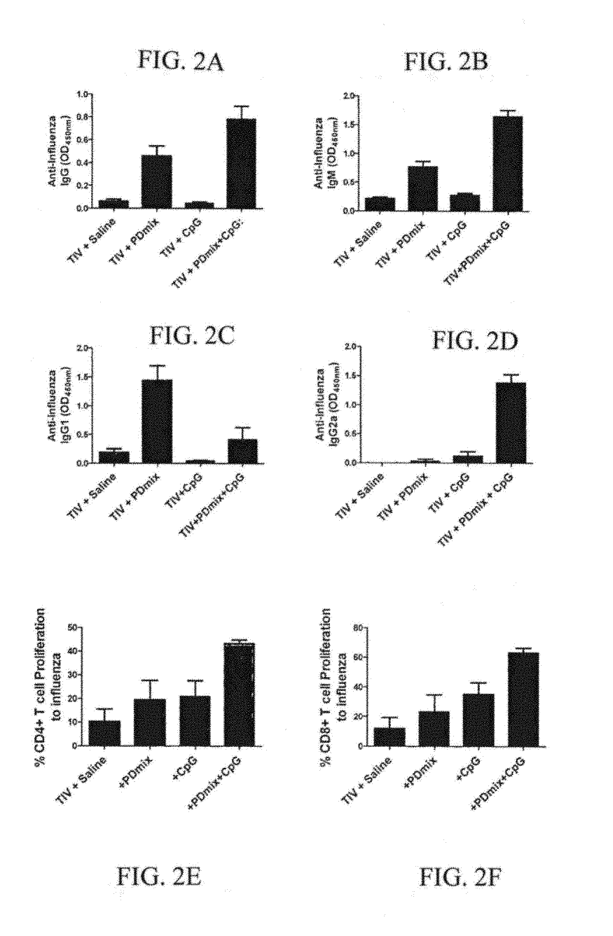

To determine whether the synergistic effect of PDmix and CpG was age-related, a similar experiment to Example 1 was undertaken using female Balb/c mice (n=10/group) that were either just 14 days old (neonatal model) or 200-300 day old (elderly model). First, 14 day old neonatal female BALB/c mice (n=5-7 per group) were immunized intramuscularly in the hindlimb with 50 μl of trivalent inactivated influenza vaccine (TIV) (Fluvax® 2007, CSL Australia) representing a dose of 100 ng HA per animal. TIV was administered alone or mixed with dIN 1 mg, PDmix (1:36 PD:D w/w) 1 mg, CpG1668 20 ug, or PDmix (1:36 PD:D w/w) 1 mg+CpG1668 20 ug. Mice were immunized twice, nine days apart and blood samples collected 14 days after the second immunization for measurement of anti-influenza antibody responses by ELISA (FIGS. 2A-2F). The addition of CpG1668 to TIV did not increase influenza-specific total IgG over that seen with influenza antigen alone, although it did result in a switch from an IgG1-predominant to an IgG2a-predominant antibody response, consistent with TLR9 agonists causing a Th2 to Th1 switch in the immune response. Maximal enhancement of anti-influenza total IgG levels was seen when the TIV was formulated with CpG1668 plus PDmix, with a synergistic effect reflected in marked enhancement of anti-influenza total IgG and IgM, to levels greater than those seen with TIV with each of the CpG1668 or PDmix alone. Only the mice that received PDmix together with CpG had a significant increase in influenza haemagglutination inhibition (HI) titers when compared to mice receiving TIV alone. Fifty two days after the second immunization the mice were sacrificed and influenza-specific T-cell recall responses measured with a CSFE-based T-cell proliferation assay. The mice that received PDmix plus CpG1668 had the highest overall CD4 and CD8 T-cell proliferative responses to influenza antigen. Hence the combination of PDmix, an inulin particle formulation, and CpG, a PAMP innate immune activator that activates TLR9, provided a synergistic enhancement of the immune response to TIV, generating the highest overall anti-influenza total IgG and IgM, being the only group to induce high levels of IgG2a, and increasing protective hemagglutination inhibition (HI) titers in the neonatal mice. Similarly, CD4+ and CD8+ T-cell proliferative recall responses to influenza antigen were also greatest in the combination group. This indicates that the combination of inulin particles and a TLR9 agonist is particularly beneficial in the induction of humoral and cellular immune responses in neonates.

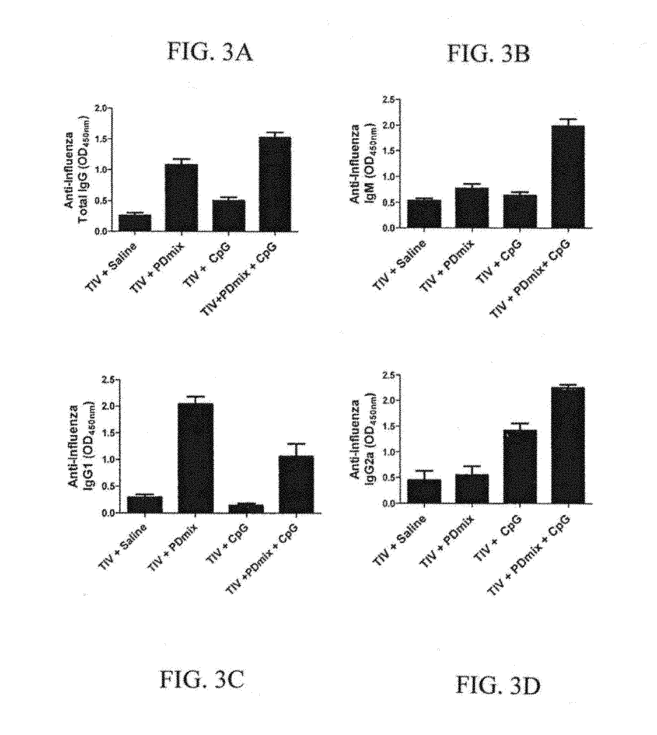

Elderly mice that were 200-300 days old (n=6/group) were immunized intramuscularly twice 14 days apart with TIV (100 ng HA) with or without 1 mg PDmix (1:36), 20 ug CpG1668 or a mixture of the two. Mice were immunized twice, 14 days apart and blood samples collected 14 days after the second immunization for measurement of anti-influenza antibody responses by ELISA (FIGS. 3A-3D). The synergistic effects of co-administration of PDmix and CpG1668 on the adaptive immune response were again observed in elderly mice with the group co-administered TIV plus PDmix inulin particles plus the TLR9 agonist CpG2006 achieving the highest influenza-specific total IgG, IgG2a and IgM responses and with the inulin particles attenuating the normal suppression of IgG1 production seen with CpG alone.

### Example 3

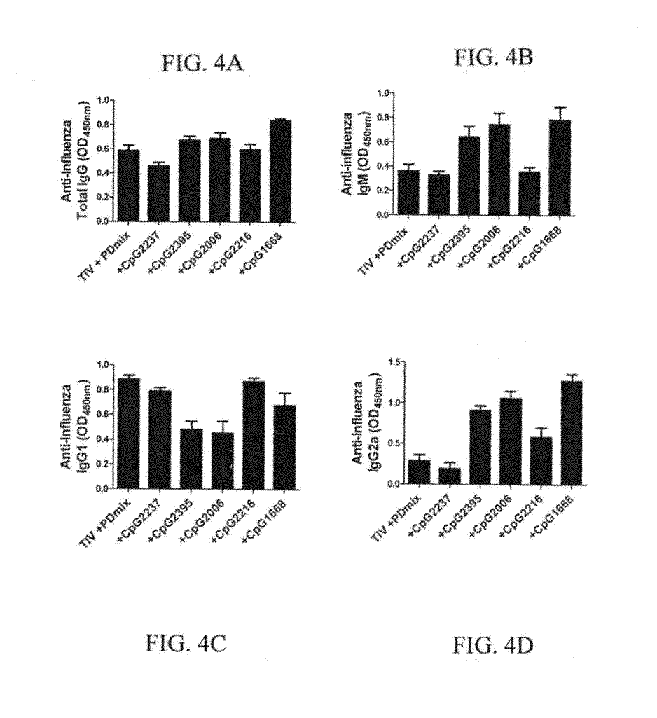

To determine whether the synergistic effect of PDmix and CpG was dependent on the sequence of the CpG, the experiment in Example 1 was repeated using 6-8 weeks old female Balb/c mice (n=5-7 per group) immunized intramuscularly twice 14 days apart. Mice were immunized intramuscularly with TIV 100 ng HA plus 1 mg PDmix (1:3) alone or together with CpG1668 (Class B ODN), CpG2216 (A class ODN), CpG2006 (Class B ODN), CpG2395 (C class ODN) or a control non-CpG sequence CpG2237, all at a dose of 10 nmol per mouse. Sequences were as follows, CpG1668-tccatgacgttcctgatgct; CpG2216-ggGGGACGATCGTCgggggG; CpG2006-tcgtcgttttgtcgttttgtcgtt: CpG2395-tcgtcgttttcggcgcgcgccg; CpG2237-tgctgcttttgtgcttttgtgctt where lowercase letters represent phosphodiester linkages and uppercase letters represent phosphorothiorate linkages. Anti-influenza antibody levels were determined by ELISA on blood collected 28 days after the second immunization (FIGS. 4A-4D) The co-administration of PDmix with either CpG1668, CpG2006 or CpG2395 all showed synergy over the individual components in increasing anti-influenza total IgG, IgG2a and IgM titers. CpG2216 and CpG2237 had no effect on the antibody response. This confirms that the synergistic effect of inulin particles and ODN is generalizable to ODN sequences containing a TLR9-binding CpG motif, preferentially belonging to Class B or Class C ODN sequences.

### Example 4

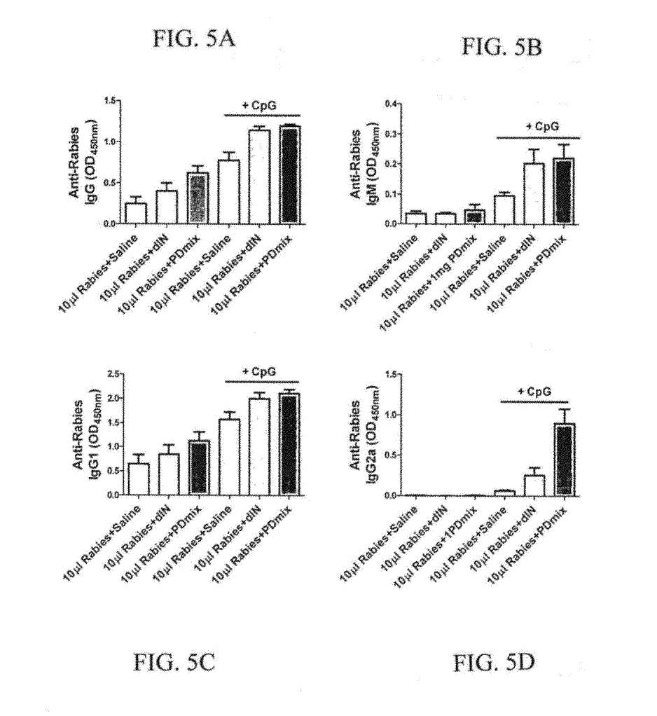

To determine whether the synergistic effect of inulin particles (dIn or PDmix) and CpG ODN was dependent on the antigen used, immunizations were repeated with an inactivated rabies vaccine (Merieux Inactivated Rabies Vaccine (MIRV). Female BALB/c mice at 6-8 weeks of age (n=5-7 per group) were immunized intramuscularly twice 14 days apart, with 10 ul of MIRV alone or combined with 1 mg of either dIN or 1 mg PDmix(1:5) alone, or mixed together with CpG1668 (5 μg). Anti-MIRV antibody levels were determined by ELISA on blood collected 14 days after the second immunization (FIGS. 5A-5D). The combination of either dIN or PDmix with CpG1668 plus MIRV provided the highest anti-rabies total IgG, IgG1, IgG2a and IgM, confirming that the synergistic effect is generalizable to both forms of inulin particles with or without alum content, and the favorable synergistic combination of inulin particles and a TLR9 agonist innate immune activator is generalizable to antigens other than influenza. Similar, studies performed in the same manner as the above experiment, confirm that the synergistic immune enhancement effect of inulin particles with CpG ODN extends to a broad range of vaccine antigens, including malaria MSP4 or MSP proteins, recombinant or inactivated SARS CoV antigen, pandemic influenza H5N1 antigen, and Japanese encephalitis antigen, with a consistent finding of enhancement of total IgG, IgG2a and IgM and attenuation of the typical suppression of IgG1 mediated by TLR9 agonists.

### Example 5

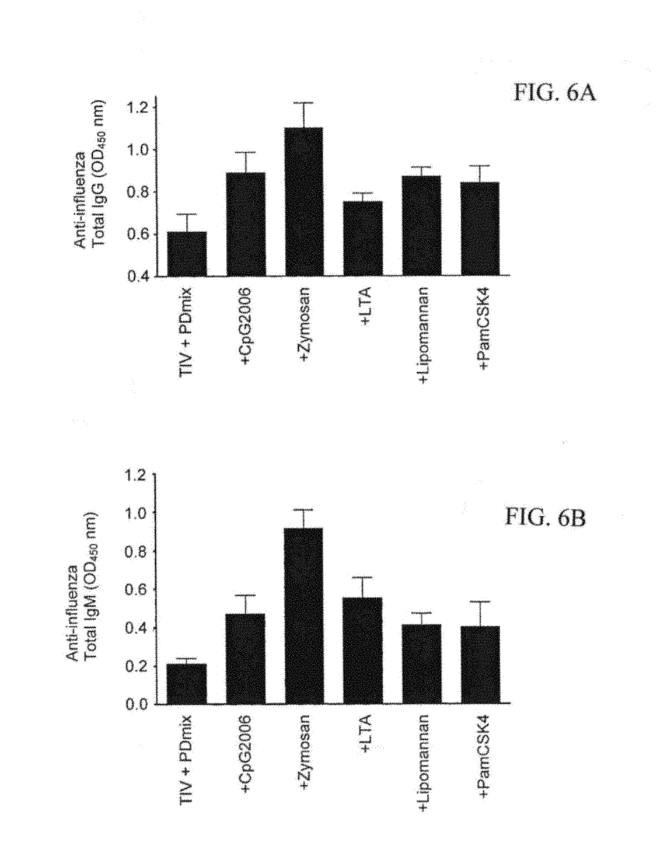

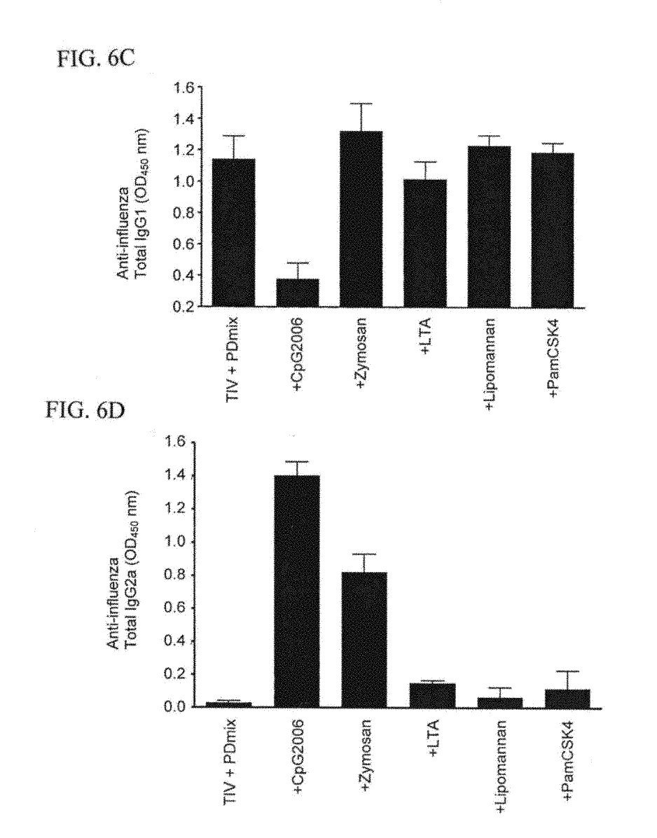

To determine whether the favorable synergistic effect of inulin particles was generalizable to other PAMP innate immune activators, female Balb/c mice at 6-8 weeks of age (n=5-10 per group) were immunized intramuscularly twice 14 days apart, with TIV 2007 (45 ng total HA/mouse) on Day 0 and Day 14. Groups received TIV plus PDmix(1:5) alone or together with 20 ug CpG2006, or one of a range of TLR2 agonists including 1 mg Zymosan, 2 ug lipoteichoic acid (LTA), 0.25 ug Lipomannnan or 0.25 ug Pam3CSK4. Sera were collected 2 weeks after the 2nd injection for measurement of anti-influenza antibodies by ELISA. (FIGS. 6A-6D). The addition of each of the individual PAMPs to the inulin particle-TIV formulation resulted in increased anti-influenza total IgG, with the greatest effect from the combination of either CpG a TLR9 agonist or zymosan, a TLR2 agonist. Whereas the combination with CpG suppressed the IgG1 response the combination with zymosan enhanced the IgG1 response, whereas both CpG and zymosan when added to inulin particles markedly enhanced the IgG2a and IgM response, with LTA and PamCSK and lipomannan also enhancing the anti-influenza IgG2a and IgM responses, albeit to a lesser degree. This showed that the synergistic immunological effect of inulin particles with TLR9 agonists extended to other PAMPs, including a range of agonists of TLR2.

### Example 6

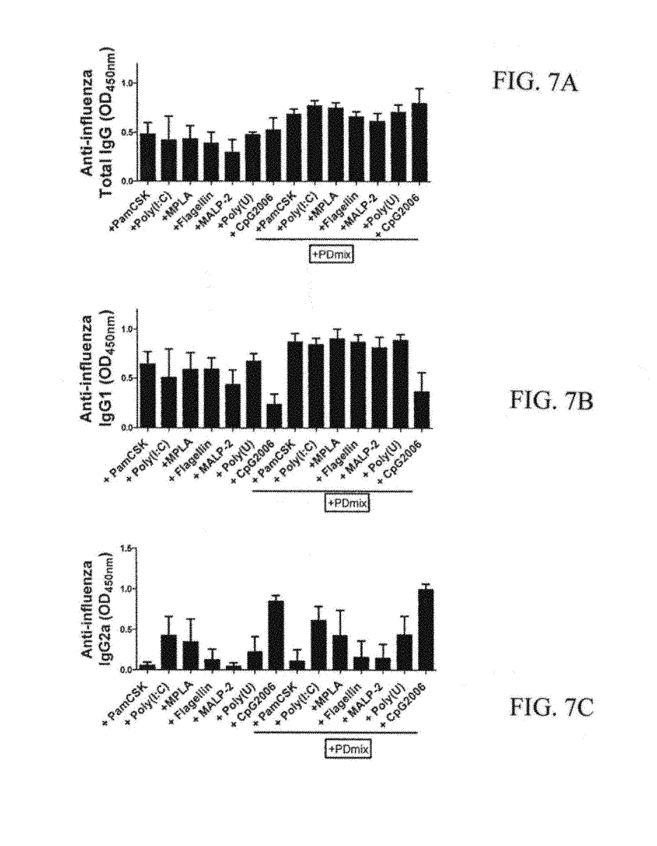

To determine whether the favorable synergistic effect of inulin particles was generalizable to yet other PAMP innate immune activators, female Balb/c mice at 6-8 weeks of age (n=5-10 per group) were immunized intramuscularly twice 14 days apart, with 1 μg recombinant yeast hepatitis B surface antigen (HBsAg) which was combined with either the TLR2 agonist PamCSK4 0.1 μg/mouse, the TLR3 agonist Poly(I:C) 25 μg/mouse, the synthetic TLR4 agonist MPLA, the TLR5 agonist flagellin, the TLR6 agonist MALP-2 0.04 μg/mouse, the TLR7 agonist PolyU 2.5 μg/mouse, or the TLR9 agonist CpG2006 20 μg/mouse, with or without 1 mg PDmix (1:3). Mice were bled 42 days after the second immunization and anti-HBsAg antibodies measured by ELISA. (FIGS. 7A-7C). The groups receiving HBsAg plus each of the PAMP innate immune activators alone had low or unmeasurable anti-HBsAg total IgG, IgG1, IgG2a and IgM. By contrast, the groups that received each of the PAMP immune activators plus PDmix showed a marked enhancement in anti-HBsAg total IgG responses consistent with a synergistic effect between the inulin particles and the PAMP innate immune activators tested

### Example 7

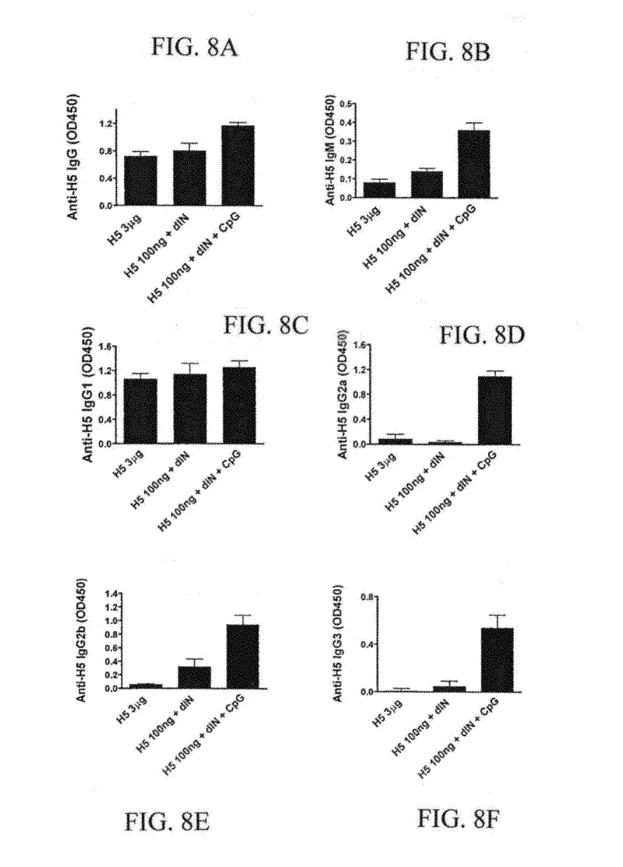

Balb/c mice at 6-8 weeks of age (n=5-8/group) were immunized intramuscularly twice 21 days apart, with 50 μl of a vaccine formulation containing between 3 ng and 3 μg of influenza recombinant H5 (rH5) serotype hemagglutinin protein (rH5) (Protein Sciences Corp, Meriden, USA) plus either dIN 1 mg or dIN 1 mg mixed with CpG2006 5 μg. Mice were bled 14 days after the second immunization and anti-recombinant H5 antibodies measured by ELISA (FIGS. 8A-8F). The results showed that when combined with dIN 1 mg plus CpG2006 5 μg just 10 ng of rH5 induced a higher IgG response than 3 μg of rH5 alone, equivalent to a greater than 300-fold antigen-sparing effect. The antigen-sparing effect was even more dramatic for the IgG2a, IgG2b and IgM responses where rH5 3 ng when combined with dIN 1 mg plus CpG2006 5 μg induced a higher IgG2a response than 3 μg of rH5 alone, equivalent to a greater than 3000-fold antigen-sparing effect

### Example 8

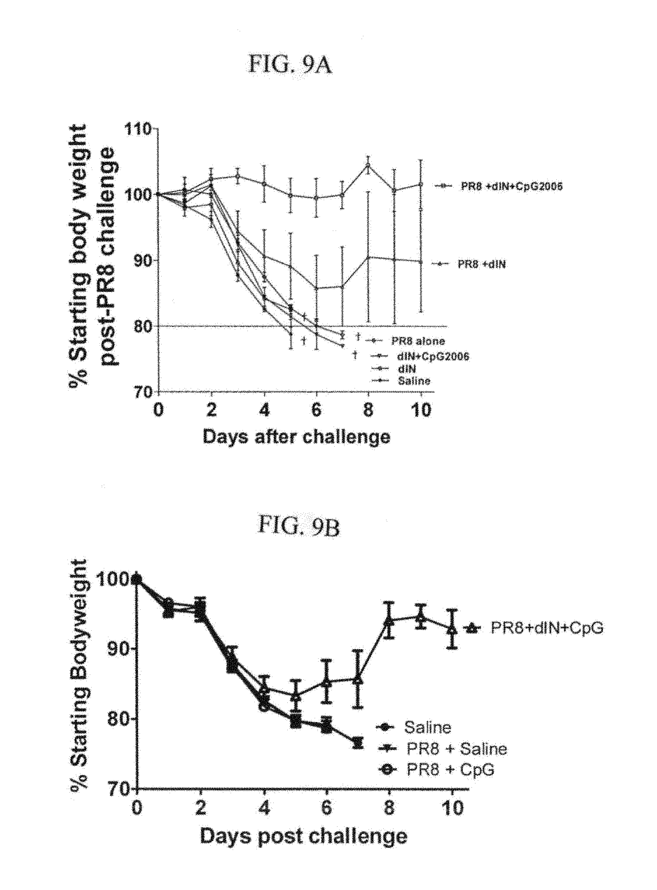

Female BALB/c mice 22 months old were immunized i.m. twice 2 weeks apart with 0.1 ug inactivated PR8 H1N1 influenza vaccine alone or combined with dIN 1 mg or dIN 1 mg+CpG2006 10 ug. Additional control groups received saline alone or dIN alone or dIN+CpG alone. All mice were then challenged intranasally at 5 weeks after the second immunization with a lethal dose of PR8 virus (20×LD50) (FIG. 9A). All control elderly mice immunized with saline or adjuvants alone and also mice immunized with PR8 vaccine without adjuvant lost weight and died. Mice immunized with PR8 vaccine plus dIN still became ill and lost weight but then recovered. By contrast elderly mice that had received PR8 vaccine plus the combination of dIN particles with CpG2006 did not become ill, lose weight or died consistent with the combination of inulin particles with a TLR9 agonist having a synergistic effect in restoring the ability of an aged immune system to respond to the vaccine and thereby obtain complete protection against clinical influenza infection. To demonstrate that the enhanced protection seen with PR8 virus challenge was not due to the CpG component by itself female BALB/c mice 6-8 weeks old were immunized i.m. with inactivated PR8 influenza antigen together with saline, CpG2006, or the combination of dIn 1 mg and CpG 10 ug at Wk 0 and 3, and mice then challenged at Wk7 with a lethal dose of PR8 H1N1 influenza virus (FIG. 9B). Only the mice immunized with PR8 plus the combination of dIn and CpG survived the challenge whereas the mice immunized with PR8 plus CpG all died, consistent with protection only being mediated by the combined presence of the inulin particles and TLR9 agonist at the time of immunization.

### Example 9

Castrated ferrets (Mustelaputoriousfuro, Triple F Farms, Sanger, Pa.) aged 11-14 weeks weighing 0.7 to 1.9 kg were held for fourteen days for acclimation and quarantine. Ferrets were seronegative for currently circulating influenza A H1 and H3, influenza B viruses, and to H5 antigen. The H5N1 A/Vietnam/1203/2004 Monovalent Influenza Subvirion Vaccine: Fisher Repository stock number—CLAG-1170 (lot#U007827) was obtained from the NIAID repository and was stored at 2 to 8° C. The vaccine was administered by intramuscular (IM) thigh injection in a volume of 0.5 mL and the other thigh for the second vaccination. Control animals received either adjuvant alone or an equal volume of buffered saline. Two formulations of inulin adjuvant were used, Lot#VAX-SPL-0910-03 (dIN inulin at 50 mg/mL in bicarbonate buffer, henceforth referred to as Ad1) and Lot# VAX-SPL-0910-04 (dIN inulin at 50 mg/mL inulin content in bicarbonate buffer mixed with CpG2006 at 0.3 mg/mL, henceforth referred to as Ad2). A dose of 250 uL per ferret of each of these formulations was mixed with the H5N1 antigen prior to immunization of each ferret. Thus ferrets received an adjuvant dose of 10 mg of dIN if randomized to receive Ad1 and an adjuvant dose of 10 mg dIn+75 μg CpG2006 if randomized to receive Ad2. CpG2006 had the sequence 5-TCGTCGTTTTGTCGTTTTGTCGTT with a complete phosphorothioate backbone and was purchased from Geneworks Pty Ltd, Adelaide, Australia. Adjuvant was stored at 2-8° C. and combined with vaccine immediately before use. Influenza virus A/Vietnam/1203/2004 (H5N1) (VN/1203) was obtained from the Centers for Disease Control and Prevention (CDC). Animals were assigned to groups using a stratified (body weight) randomization procedure by a computerized data acquisition system (e.g., Path-Tox; Xybion, Cedar Knolls, N.J.). A total of 49 ferrets were assigned to one of ten groups; Four groups of 7 ferrets each received adjuvanted vaccine twice 21 days apart: 7.5 μg vaccine+Ad1; 7.5 μg vaccine+Ad2; 22.5 μg+Ad1; 22.5 μg vaccine+Ad2. Two groups of 3 ferrets each received vaccine twice without adjuvant: 22.5 μg+No Ad; 7.5 μg+No Ad. Three control groups of three ferrets each received twice: saline+Ad1; saline+Ad2; saline+Saline. One additional group of 6 ferrets received 22.5 μg vaccine+Ad2 administered only once at the time of priming of other groups. Ferrets were infected three weeks after the vaccine booster dose, or six weeks after the priming dose in the group vaccinated only once. For the challenge procedure, following anesthesia with intramuscular ketamine (20 mg/kg) and xylazine (4 mg/kg), 106 EID50 of VN/1203 was instilled in 500 μL into each nare, and the challenge dilution was cultured to ensure consistent infections. Nasal washes were collected by instilling into each nare 1.0 mL of saline containing 1% bovine serum albumin, 100 units penicillin/mL, 100 μg/mL streptomycin, and 0.25 μg amphotericin B/mL. Whole blood for complete blood count was obtained by superior vena cava puncture on day 4 after challenge. Twice daily observations recorded ocular discharge, nasal discharge, sneezing, coughing, stool characteristics, and activity score. Moribund animals were designated by any one of the following criteria: a temperature of less than 33.3° C., weight loss >25%, unresponsiveness to touch, self-mutilation, paralysis, movement disorder, or respiratory distress. In upper respiratory tract samples obtained during life, nasal washes were obtained 2 and 4 days after viral challenge, and throat swabs were obtained 1, 2, 3, 4, and 6, days after challenge. In tissues harvested at necropsy, influenza virus was cultured from lavage of a caudal lung lobe and from four 250 mg fragments of homogenized (TissueLyser, QIAGEN, Valencia, Calif.) lung, brain, spleen, tracheobronchial lymph nodes, and two tracheal rings. Serum was collected by vena cava puncture on the day of first vaccination and 14, 21, and 28 days after first vaccination; day 14 post vaccination corresponds to day −28 before challenge, and day 28 post vaccination corresponds to day −14 before challenge. Serum samples were inactivated by receptor-destroying enzyme (Denka-Seiken, Tokyo, Japan) at 37° C. for 16-20 hours followed by heat inactivation at 56° C. for 30 minutes. Hemagglutination inhibition (HI) was performed using horse red blood cells. Titers of neutralizing antibodies were measured by the microneutralization assay (MN). One hundred tissue culture infectious dose 50 (100 TCID50) of VN/1203 virus was mixed with an equal volume of serial dilutions of serum in quadruplicate, incubated for 1 hour at 37° C. and 100 μL of the mixture was added to a prewashed monolayer of MDCK cells in 96 well plates. The plates were incubated for 3 days and the cytopathic effect (CPE) was visually assessed using an inverted microscope. The highest serum dilution protecting more than half of the wells was taken as the antibody titer. Geometric mean titers are reported and a negative titer was denoted as 10. Lung tissue and brain with olfactory bulbs were collected at necropsy from ferrets moribund on days 6 to 8 post-challenge and from surviving ferrets free of symptoms at day 14 post-challenge. After fixation in buffered formalin, standardized sections were trimmed for histopathology from the left cranial, right middle and right caudal lung lobes. Statistical analyses were performed using GraphPad Prism (version 5.03, GraphPad Software, Inc. La Jolla, Calif.). Serum antibody response was analyzed by analysis of variance (ANOVA) using the Bonferroni post-test correction. Survival proportions were tested using the Log-Rank test. Morbidity by increasing activity score was examined by Fisher's exact test. Viral load was determined to be different by the repeated measure ANOVA.

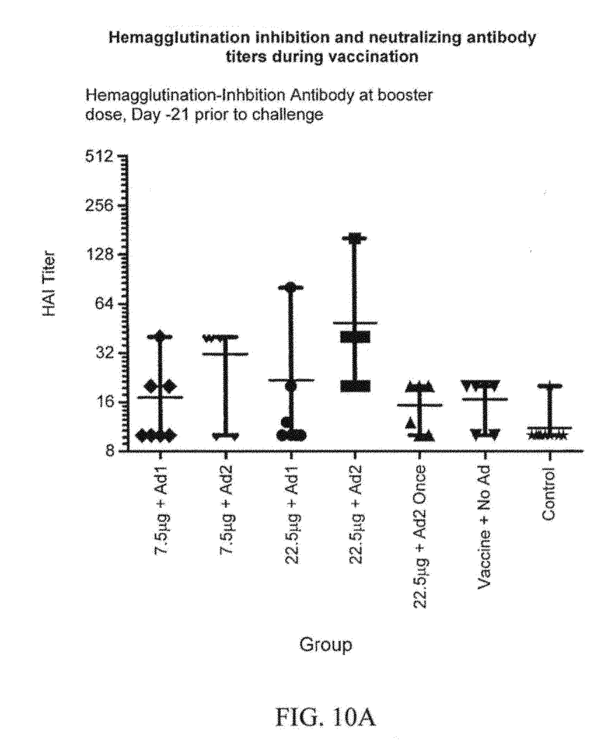

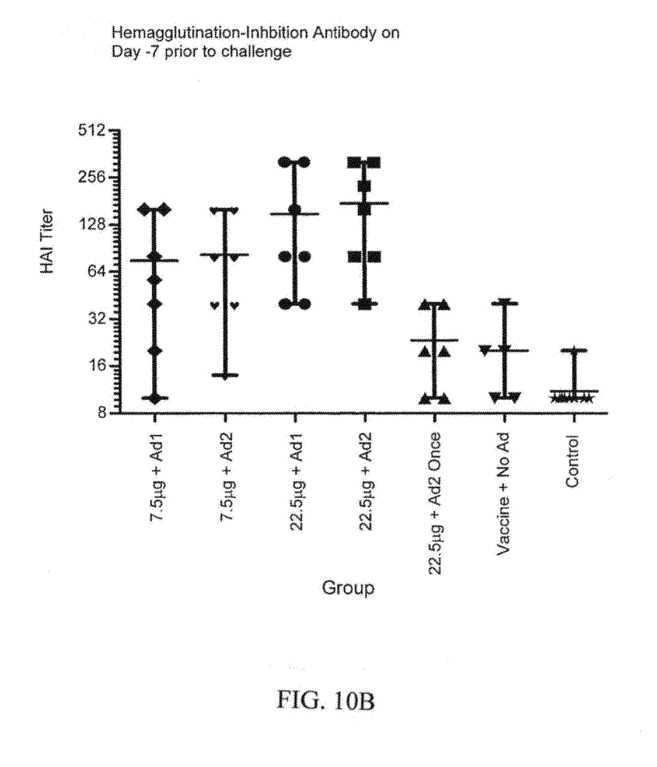

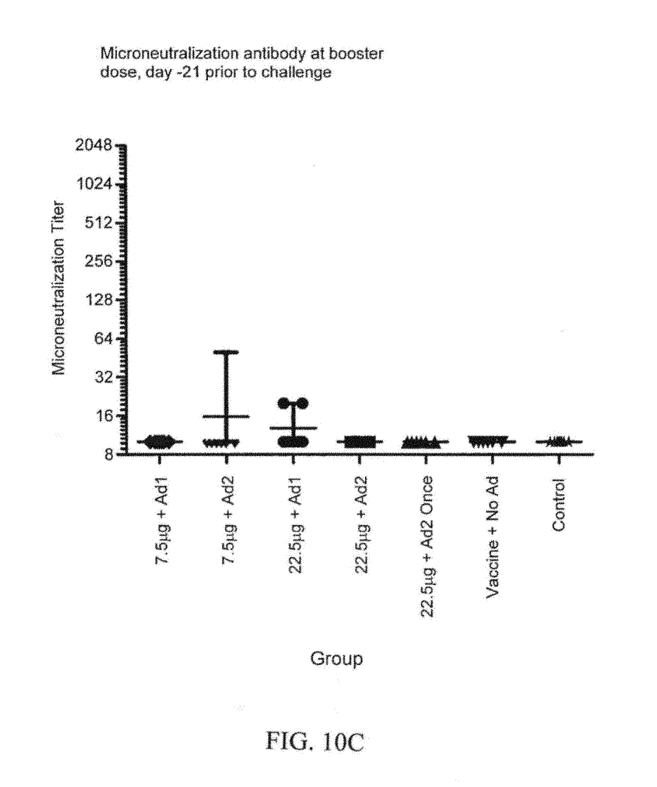

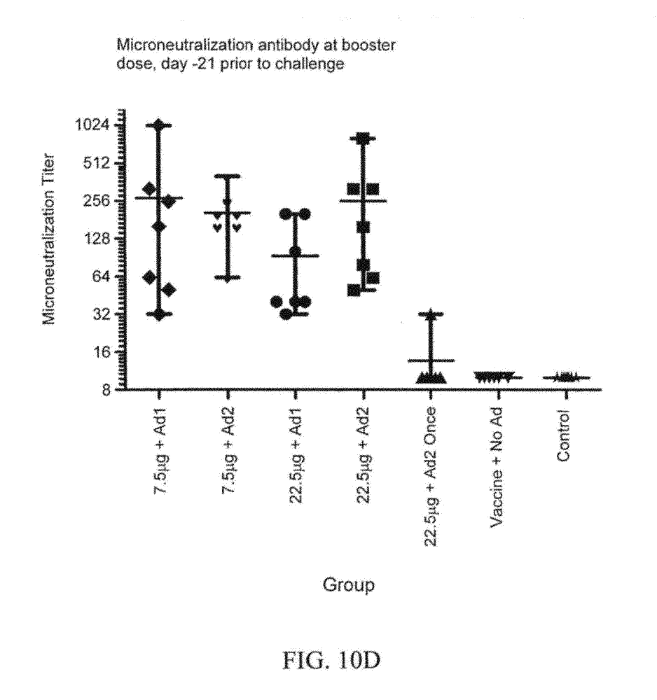

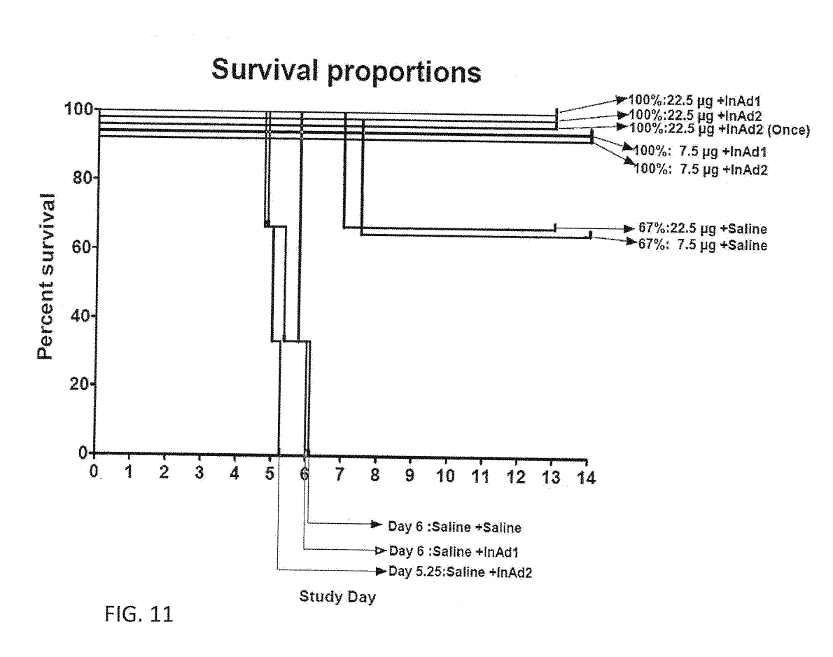

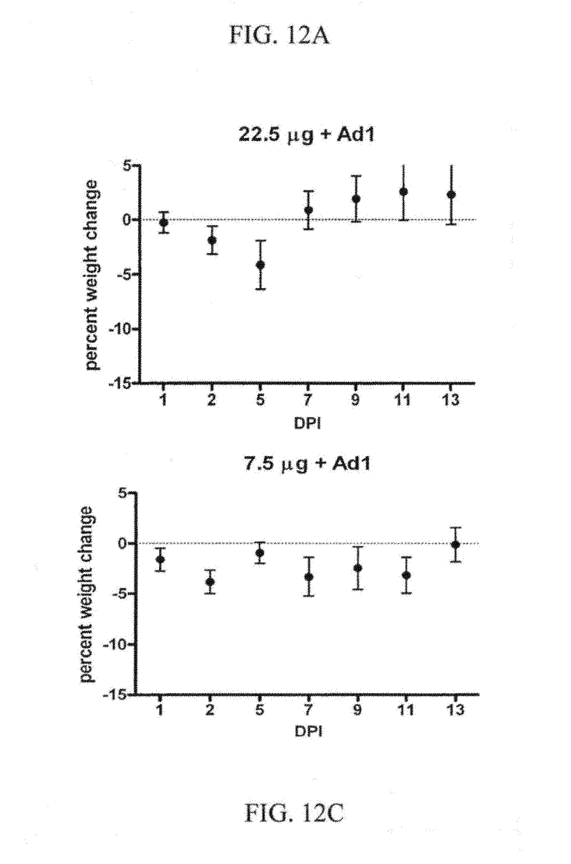

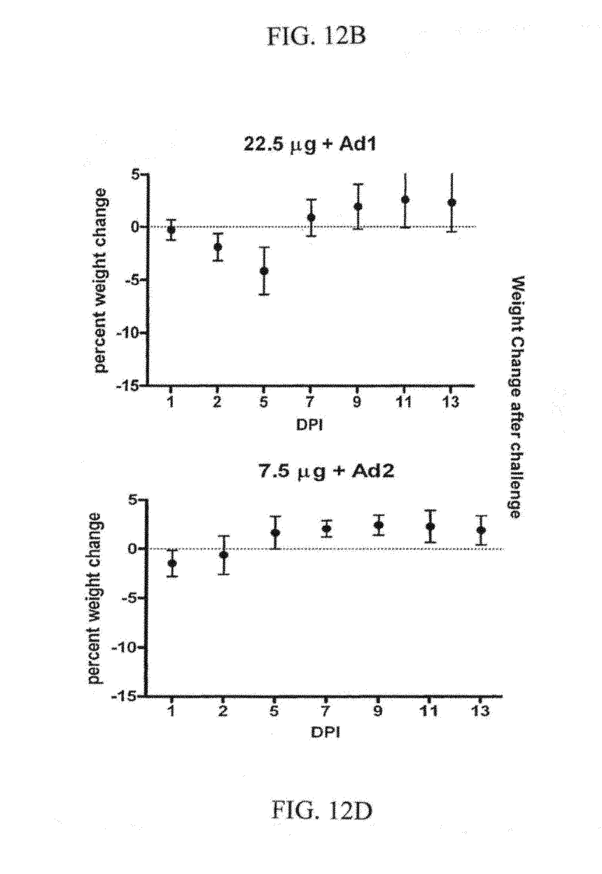

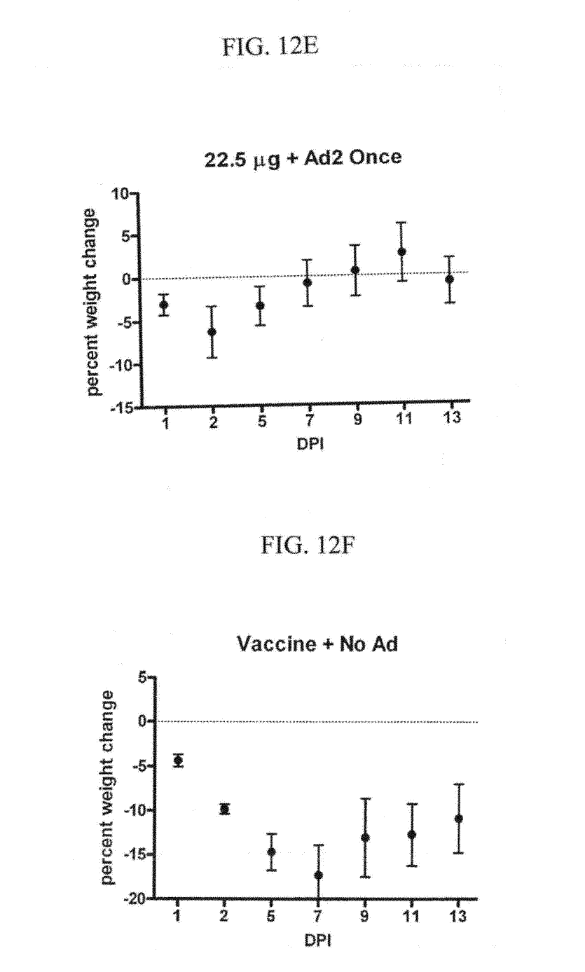

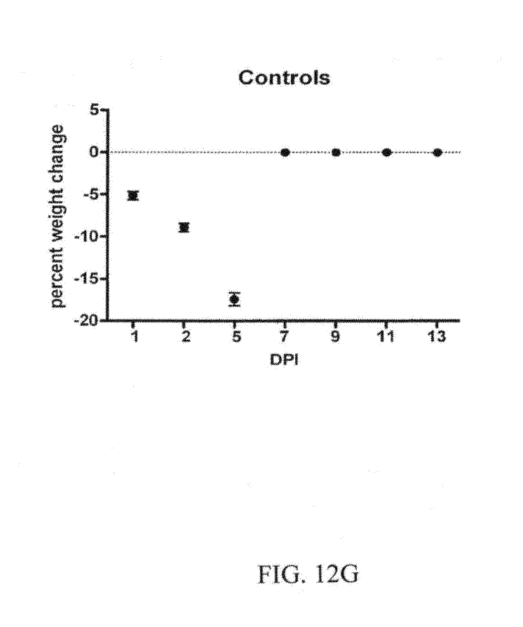

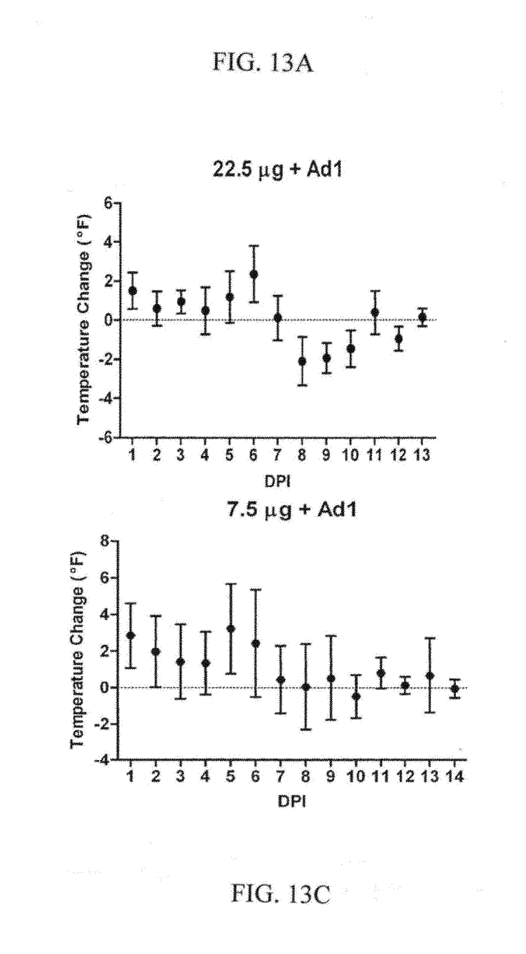

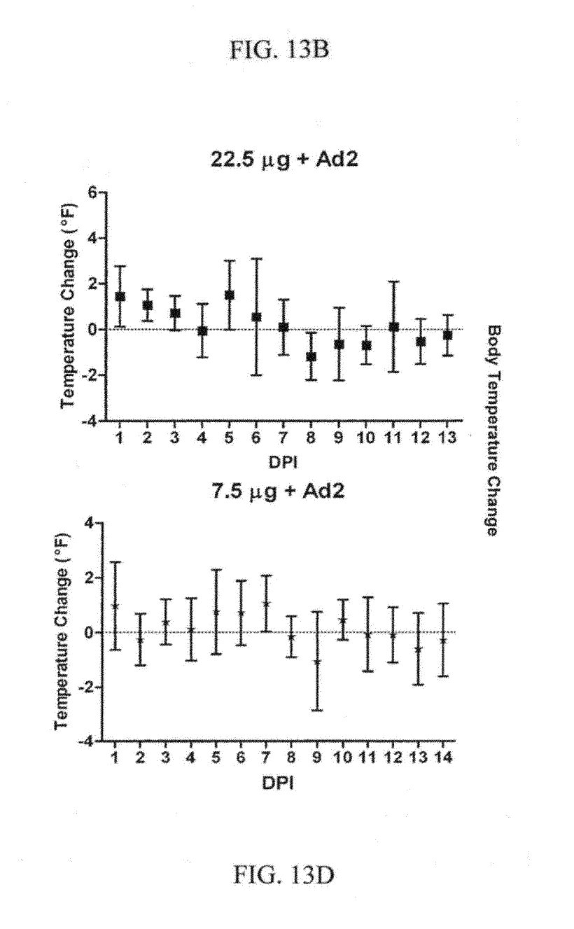

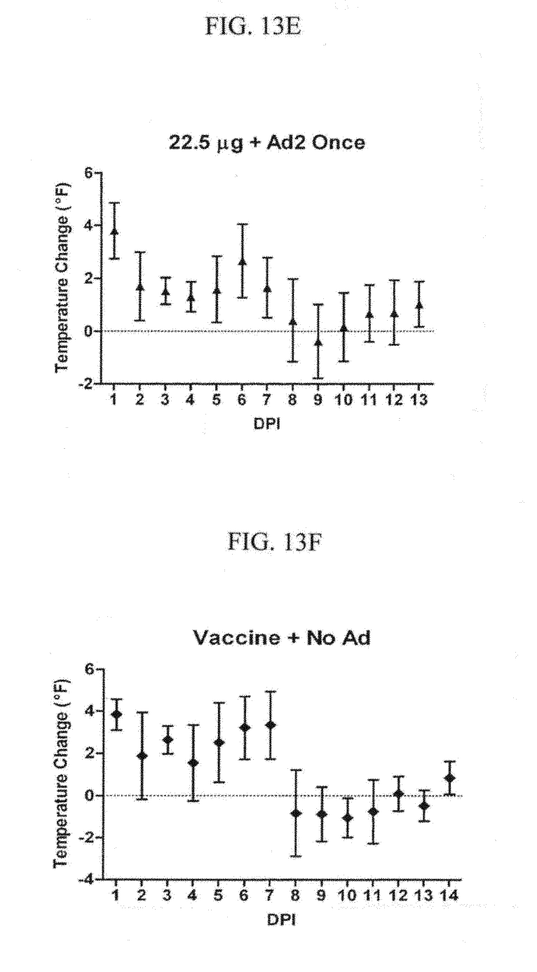

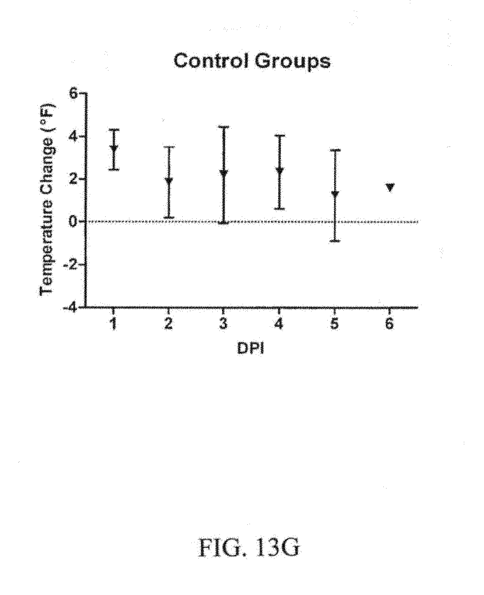

Ferrets immunized with split-virion H5N1 vaccine without adjuvant, regardless of vaccine dose, did not have detectable H5N1 neutralizing antibody prior to challenge. Ferrets receiving two doses of H5N1 vaccine with Ad1 or Ad2 all demonstrated neutralizing antibody pre-challenge and at 21 days after the priming dose, Ad2-adjuvanted vaccine recipients had significantly higher serum neutralizing antibody than the Ad1 groups (p<0.03, Log Rank-sum test), consistent with the combination of inulin particles plus a PAMP innate immune activator (CpG) providing an enhanced immune response (FIGS. 10A-10D). Control animals all died after challenge, animals vaccinated with two doses of antigen alone suffered approximately 30% mortality and no mortality was observed in animals vaccinated with antigen combined with either Ad1 or Ad2 (FIG. 11). Recipients of two doses of vaccine without adjuvant lost greater than 15% of body weight by day 5 post-immunization (pi) and the four survivors failed to recover the weight loss. While groups vaccinated with two doses of antigen with Ad1 lost 5% of body weight then recovered, groups vaccinated with two doses of antigen with Ad2 did not lose any weight, consistent with enhanced immune protection when the H5N1 antigen was combined with a formulation of inulin particles plus a PAMP innate immune activator (FIGS. 12A-12G). Similarly, while 4 ferrets in the Ad1-adjuvanted vaccine groups demonstrated fever, no ferrets in the Ad2-adjuvanted group experienced fever, consistent with a synergistic protective effect between the inulin particles and the PAMP innate immune activator (FIGS. 13A-13G). Throat swab influenza virus titers in Ad2 vaccine recipients on days 2, 3, and 4 pi were significantly lower than in antigen-alone recipients (Mann-Whitney, p=0.0018) while the titers in Ad1 vaccine recipients were not significantly different to the vaccine-alone recipients. Recipients of the single dose of vaccine with Ad2 did not have significant difference in viral loads on day 2-4 pi compared to the two dose antigen-alone groups. Thus the combination of a inulin particle formulation (dIN) with a PAMP innate immune activator (CpG2006) synergistically enhanced the antibody response to a co-administered antigen and provided enhanced protection against lethal H5N1 challenge, even after just a single immunization. Performance of similar one dose vaccine studies in mice with PR8 antigen conformed that complete protection of mice against lethal PR8 challenge could be obtained by immunizing them with a single dose of 5 ug PR8 combined with dIN and CpG2006 (10 ug), whereas immunization with PR8 with either component alone provided only partial or no protection, respectively.

### Example 10

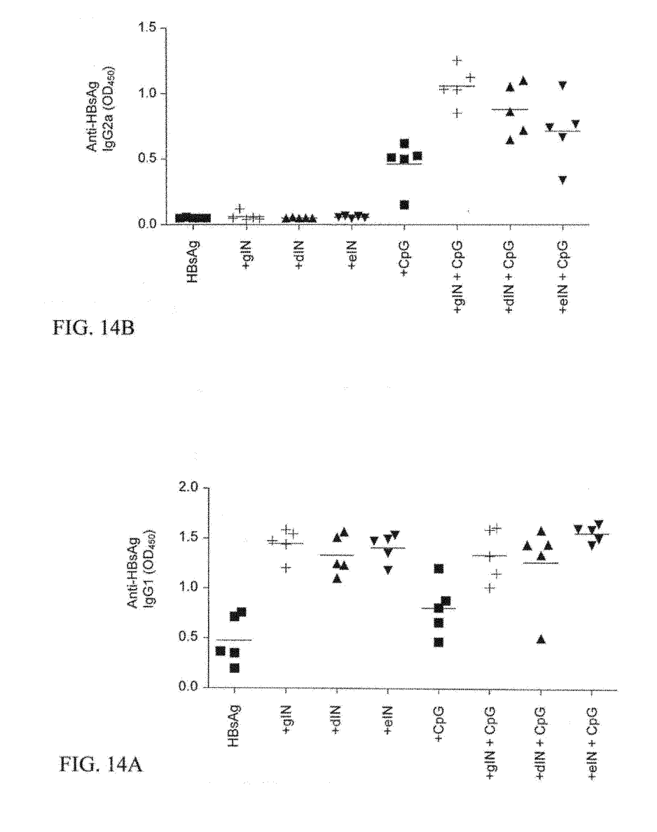

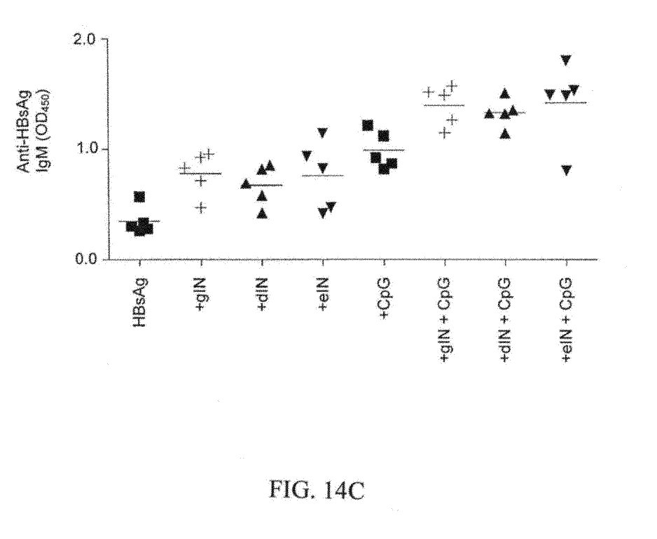

To test whether the synergistic effect of inulin particles when combined with a PAMP innate immune activator, was purely a property of dIn or was shared by other inulin particle polymorphic forms, adult Balb/c mice were immunized intramuscularly twice 21 days apart, with HBsAg together with either gIN, dIN or eIN inulin particles together with the TLR9 PAMP, CpG2006. Mice were bled 14 days after the second immunization and anti-influenza antibodies measured by ELISA. (FIGS. 14A-14C). gIN, dIN or eIN had a synergistic enhancing effect with the CpG in the induction of anti-HBsAg IgG1, IgG2a and IgM consistent with the synergistic effect on PAMP innate immune activators being a shared property of different polymorphic forms of inulin particles

### Example 11

To determine if the synergistic effects of inulin particles and a PAMP were translatable from small animal models to large mammals, groups of standard bred, female horses (n=3/group), 4-8 years of age and sero-negative to JEV, were immunized with a Japanese encephalitis (JE) vaccine by subcutaneous injections in the neck region. Vero cell culture-grown inactivated JE vaccine (ccJE; Beijing-1 strain) (Toriniwa& Komiya, 2008) obtained from the Kitasato Institute, Japan was given at a dose of 6 μg, either alone or together with a dIN inulin particle formulation (20 mg/dose) or both dIN inulin particle formulation (40 mg/dose) plus CpG7909 (200 ug/dose) in a total injection volume of 1 mL. Horses were boosted with a second dose of the same vaccine after 5-weeks, and sera were collected 5 weeks after the 1st and 2nd immunizations. 50% plaque-reduction neutralization tests (PRNT50) were performed by incubating ˜400 PFU of JEV (Nakayama strain), MVEV (MVE-1-51 stain) or WNV (Kunjin MRM61C strain) in 110 μl HBSS-BSA with serial 2-fold dilutions of antiserum in the same buffer in a 96-well tray at 37° C. for 1 h. Duplicate 0.1 mL aliquots were assayed for infective virus by plaque formation on Vero cell monolayers grown in 6-well tissue culture trays. The percentage plaque reduction was calculated relative to virus controls incubated with naïve serum from the same mouse strain. PRNT50 titers are given as the reciprocal of serum dilutions, which resulted in ≧50% reduction of the number of plaques. Comparison of PRNT50 titers against JEV after 2 doses of vaccine showed that when ccJE was formulated with inulin particles alone, the neutralizing antibody responses were augmented by ˜4-fold relative to the standard ccJE group. However, the co-administration with ccJE antigen of both inulin particles and CpG7909 resulted in a further 2-3 fold increase in JEV neutralizing antibody (Table 1). Notably, all horses receiving ccJE with inulin particles plus CpG achieved a seroprotective antibody titer (PRNT50>10) after just a single dose. The combination of inulin particles with the TLR9 agonist also resulted in the highest level of cross-neutralizing antibodies against MVEV and WNV, indicating that this combination is particularly favorable for the induction of cross-neutralizing antibodies against other virus strains or even other viruses entirely.

TABLE 1

| | | | MVEV | WNV |
|---|---|---|---|---|
| | JEVPRNT₅₀ | JEV PRNT₅₀ | PRNT₅₀ | PRNT₅₀ |
| | Post-prime | Post boost | Post boost | Post boost |
| Vaccine | (GMT) | (GMT) | (GMT) | (GMT) |
| ccJE | 11 | 168 | 40 | <10 |
| ccJE + dIN | 14 | 635 | 50 | 21 |
| ccJE + dIN + CpG | 43 | 1600 | 126 | 40 |

### Example 12

The anti-inflammatory effects of inulin particles can be conveniently measured by an assay using human whole blood or purified human peripheral blood mononuclear cells (PBMC) or in the alternative if preferred in mouse or other small species by using purified splenocytes or if the animal is larger e.g., a rabbit, by similarly using their whole blood or purified peripheral blood mononuclear cells. In summary, a titration series of a reducing concentration of the inulin particles, from 1 mg/mL down to 1 ng/mL are added to the cells in a multiwell pate which is then incubated at 37 C or the relevant body temperature of the species from which the cells were obtained. The readout is by measurement of cytokines with IL-1 being especially preferred. The readout can be made after between 4 and 24 hours if cytokine gene expression is being measured by real time PCR or after between about 24 and 72 hours if cytokine protein production is being measured, for example by ELISA. For this example, human PBMC were prepared from 3 healthy adult human subjects and incubated with 100 ug/mL of dIN particles for 5 hours after which the RNA was extracted with TRIZOL and then run on a gene expression array system (Illumina). For control comparison purposes, PBMC from the same subjects were incubated with pro-inflammatory PAMPs including poly(I:C) and LPS. As expected IL-1α and IL-1β mean gene expression across the three human subject PBMC was upregulated by a mean of 4.1 and 4.4 fold, after incubation of PBMC from the 3 subjects with Poly(I:C) or LPS, respectively, when compared to PBMC incubated in the absence of the PAMP agonist. By contrast, IL1α gene expression was reduced 2.88 fold and IL1β gene expression 2.17 fold in PBMCs cultured with dIN particles 100 ug/mL when compared to PBMC incubated alone. dIN particles also downregulated IL1 receptor gene expression, namely IL1RAP which was 1.46 fold downregulated in the presence of inulin particles. Furthermore, further emphasizing their anti-inflammatory action, dIN particles resulted in upregulation of genes that antagonize the inflammatory action of IL-1 including IL1F5 (1.49 fold upregulated), IL1R2 (1.11 fold upregulated), and IL1RN (2.9 fold upregulated). Next the effect of the combination of dIN particles and the TLR9 agonist PAMP, CpG, was examined. In the presence of dIN particles plus CpG, IL1α and IL1β gene expression remained downregulated when compared to expression in unstimulated PBMC alone, but interestingly in the presence of the combination of dIN and CpG the gene expression of IL1 antagonists was even more greatly upregulated than in the presence of dIN alone. Hence with the combined stimulation the effect on genes that antagonize the inflammatory action of IL-1 including IL1F5 (dIN alone vs dIN+CPG) was (1.9 vs 1.49 fold upregulated), IL1R2 (1.35 fold vs 1.11 fold upregulated), and IL1RN (3.47 fold 2.94 fold upregulated). Thus, even more surprisingly the combination of inulin particles with the TLR9 agonist PAMP resulted in even greater enhancement of the anti-inflammatory properties of the inulin particles alone. Conversely, in the same assay genes associated with anti-inflammatory effects were consistently elevated. Thus, the anti-inflammatory gene, PPARg, was consistently downregulated in PBMC incubated with PAMCSK, poly(I:C), LPS and all other TLR agonists tested, but was upregulated by a mean of 1.24 fold when PBMC from the three human subjects were incubated with dIN particles. Matching results were obtained when proteins levels of the same and related pro-inflammatory markers were measured in human PBMC after 24-48 hours incubation with a PAMP, or inulin particles, with protein levels being measured by cytokine ELISA or by Western blot. The results showed that expression of PAMP-stimulated inflammatory cytokines including IL-1 by human PBMC incubated with whole live or inactivated virus (JEV) or purified PAMPs, is reduced in the presence of inulin particles in the PBMC cultures. gIN and eIN particles showed identical effects to dIN in respect of their ability to inhibit IL-1 gene and protein expression and to upregulate expression of anti-inflammatory members of the IL1 pathway, and PPARγ, making this a generalizable property of all inulin particles tested.

As part of a human H1N1 2009 pandemic influenza vaccine study, dIN was administered to human subjects in a dose of 20 mg per immunization combined with a recombinant H1N1 2009 haemagglutinin antigen (rHA). The frequency of headache was significantly lower (p<0.05 by Fishers exact test) in subjects receiving Advax™ adjuvant (4/137: 2.9%), compared to rHA alone (15/137: 10.9%). After the second immunization there was again a trend (p=0.06) to less post-immunization headaches in groups receiving Advax™ adjuvant (2/135: 1.5%) compared to rHA alone (8/137: 5.8%). Reduction in headaches would be consistent with inulin particle-induced inhibition of IL-1 production, as IL-1 serum levels are increased in cluster headaches and IL-1 gene polymorphisms (3953 C/T) are associated with migraine headaches (Martelletti et al., 1993; Rainero et al., 2002). This indicates at a proven adjuvant-effective dose in humans, inulin particles are also having an anti-inflammatory effect.

### Example 13

To determine whether the favorable synergistic effect of inulin particles was generalizable to yet other PAMP innate immune activators, female Balb/c mice at 6-8 weeks of age (n=5-10 per group) were immunized intramuscularly twice 14 days apart, with 1 μg recombinant yeast hepatitis B surface antigen (HBsAg) which was combined with either the TLR2 agonist PamCSK4 0.1 μg/mouse, the TLR3 agonist Poly(I:C) 25 μg/mouse, the synthetic TLR4 agonist MPLA, the TLR5 agonist flagellin, the TLR6 agonist MALP-2 0.04 μg/mouse, the TLR7 agonist PolyU 2.5 μg/mouse, or the TLR9 agonist CpG2006 20 μg/mouse, with or without 1 mg PDmix (1:3). Mice were bled 42 days after the second immunization and anti-HBsAg antibodies measured by ELISA. The groups receiving HBsAg plus each of the PAMP innate immune activators alone had low anti-HBsAg total IgG, IgG1, IgG2a and IgM. By contrast, the groups that received each of the PAMP immune activators plus PDmix showed a marked enhancement in anti-HBsAg total IgG responses consistent with a synergistic effect between the inulin particles and the PAMP innate immune activators tested.

### Example 14

Design of an Epitope Vaccine

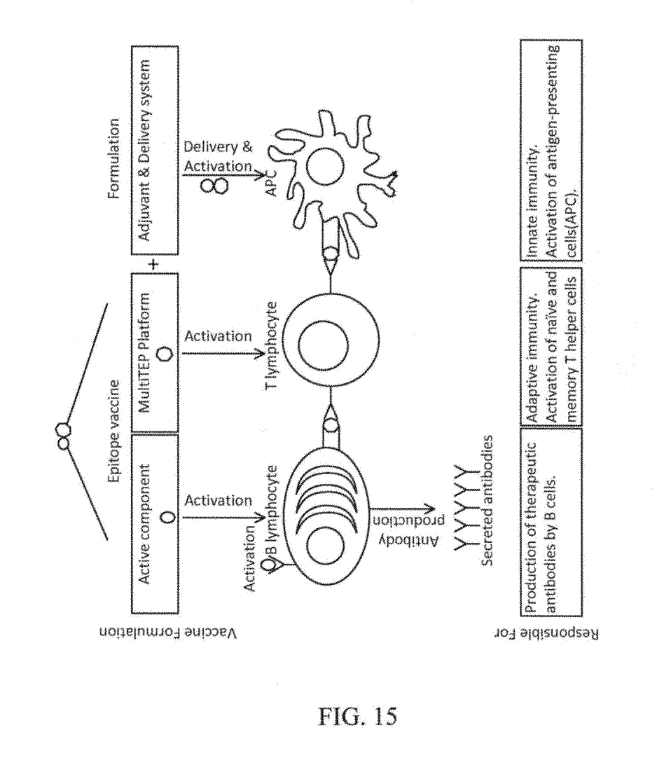

The design of the epitope vaccine compositions is based on a platform of multiple promiscuous T helper (Th) foreign epitopes (MultiTEP). The mechanism of action for MultiTEP-based epitope vaccine is shown in FIG. 15. MultiTEP component of vaccine activates an adaptive immunity providing a broad coverage of human MHC polymorphism and activating both naive T cells and pre-existing memory T cells generated in response to conventional vaccines and/or infections with various pathogens during lifespan. The MultiTEP platform fused with any B cell epitope or combination of epitopes from Aβ, tau, or α-syn induces production of therapeutic antibodies.

### Example 15

Immunogenicity and Efficacy of DNA-Based MultiTep Epitope Vaccines in Mice, Rabbits, and Monkeys.

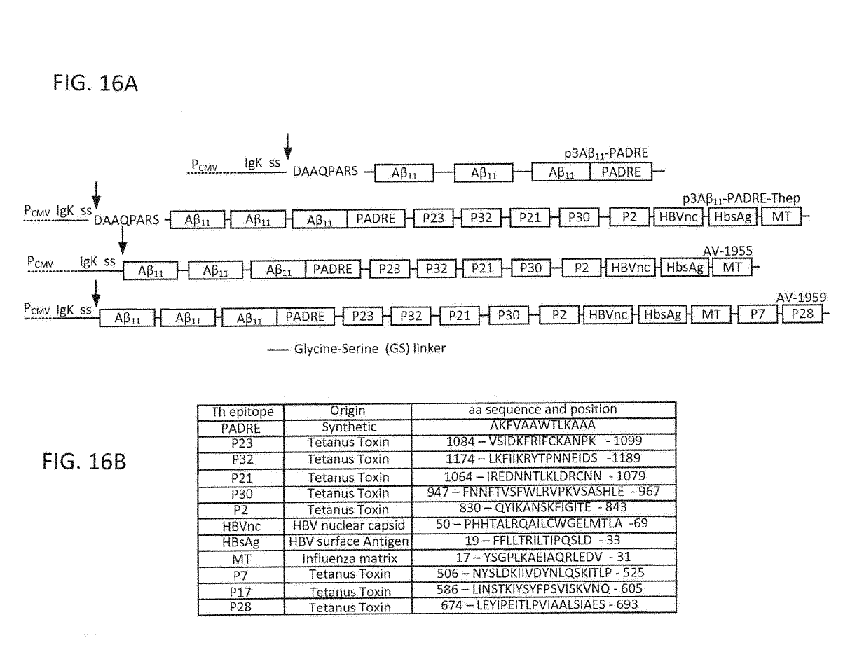

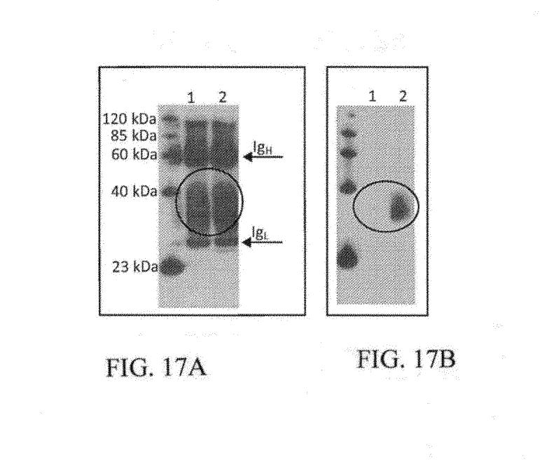

In this example, modified versions of the p3Aβ₁₁ PADRE vaccine are engineered to express p3Aβ₁₁ possessing a free N-terminal aspartic acid in the first copy and fused with PADRE and eight (AV-1955) or eleven (AV-1959) additional promiscuous Th epitopes designated collectively as MultiTEP platform. The construction strategy of p3Aβ₁₁-PADRE has been described (Movsesyan N, et al. PLos ONE 2008 3:e21-4; Movsesyan N, et al. J Neuroimmunol 2008 205:57-63)). A polynucleotide encoding multiple T helper epitopes (MultiTEP) separated by GS linkers is synthesized and ligated to the 3Aβ₁₁ PADRE minigene (FIGS. 16A-16B). Correct cleavage of signal sequence and generation of N-terminus aspartic-acid in first copy of Aβ₁₁ was shown by IP/WB techniques (FIGS. 17A-17B).

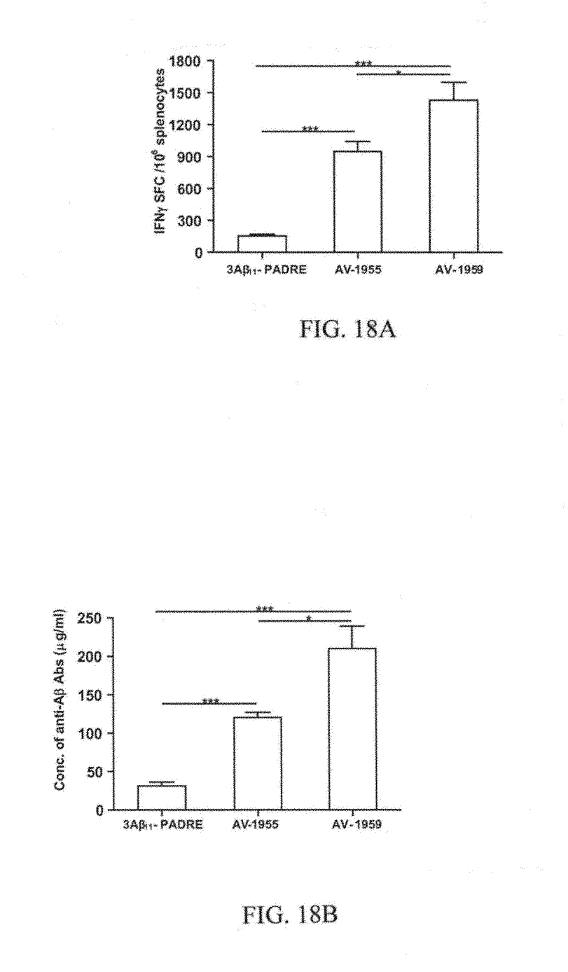

The immunogenicity of MultiTEP-based DNA epitope vaccines is established in mice after delivery by gold particles using a gene-gun device. As shown, cellular (FIG. 18A) and humoral (FIG. 18B) immune responses induced by MultiTEP vaccines AV-1959 and AV-1955 are significantly higher than responses obtained from delivery of a first generation epitope vaccine, which has only PADRE Th epitope.

Immunogenicity of MultiTep vaccines was also tested in mice, rabbits and monkeys after electroporation-mediated needle delivery. Mice, rabbits and monkeys were immunized several times biweekly or by monthly injections of DNA vaccine followed by electroporation. Blood was collected 12-14 d after each immunization. In all tested species, MultiTep DNA vaccine induces strong cellular immune responses specific to foreign Th epitopes (MultiTep platform) but not to Aβ₁₁ or Aβ₄₀ (data not shown).

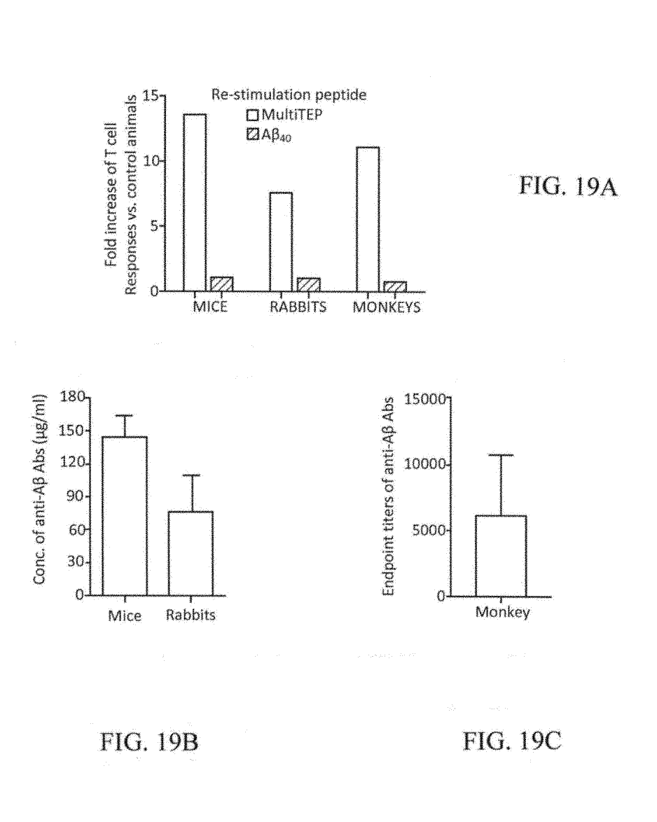

Splenocytes of mice and PBMC of rabbits and monkeys were re-stimulated in vitro with recombinant protein containing only the Th epitope portion of the vaccine, with a cocktail of individual peptides presenting Th epitopes, or with the Aβ₄₀ peptide. Both protein and the peptides cocktail induced equally strong in vitro proliferation and IFNγ production by splenocytes and PBMC of immunized, but not control animals; in contrast, no proliferation or IFNγ production was observed after re-stimulation with Aβ40 peptide in splenocytes or PBMC of either immunized or control animals (FIG. 19A and data not shown). The data show that activated Th cells helped B cells to produce high amount of Aβ specific antibodies.

The concentrations (in sera from mice and rabbits) and titers (in sera from monkeys) of anti-Aβ antibodies were determined by standard ELISA. Both MultiTEP platform based DNA vaccines (AV-1955 and AV-1959) induced strong cellular and humoral immune responses in mice (including APP/tg mice, data not shown), rabbits and monkeys. Concentration and endpoint titers of antibodies generated by AV-1959 DNA epitope vaccine are presented in FIGS. 19B and 19C.

Antibodies generated in all species were therapeutically potent. Anti-Aβ₁₁ antibodies were purified from sera of mice, rabbits or monkeys immunized with DNA epitope vaccine by an affinity column (SulfoLink, Pierce, Rockford, Ill.) immobilized with Aβ18-C peptide (GenScript, Piscataway, N.J.) as previously described (Mamikonyan G, et al. J Biol Chem 282:22376-22386, 2007). Purified antibodies were analyzed via electrophoresis in 10% Bis-Tris gel, and the concentrations were determined using a BCA protein assay kit (Pierce, Rockford, Ill.).

Therapeutic potency of purified antibodies was analyzed in vitro and ex vivo by a neurotoxicity assay (Mamikonyan G, et al. J Biol Chem 282:22376-22386, 2007; Ghochikyan A, et al. Hum Vaccin Immunother 9:1002-1010, 2013; Davtyan H, et al., J Neurosci 33:4923-4934, 2013) and binding to Aβ plaques in human brain tissues. Sera from immunized animals were screened for the ability to bind to human Aβ plaques in 50 μm brain sections of formalin-fixed cortical tissue from an AD case (received from the Brain Bank and Tissue Repository, MIND, UCI, Irvine, Calif.) using standard immunohistochemistry.

Evaluation of antibodies to Aβ, Binding of antibodies to different forms (e.g., monomeric and aggregated forms) of Aβ₄₂ peptide were performed on a BIAcore 3000 SPR platform (GE Healthcare, Piscataway, N.J.) as described (Mamikonyan G, et al. J Biol Chem 282:22376-22386, 2007; Ghochikyan A, et al. Hum Vaccin Immunother 9:1002-1010, 2013; Davtyan H, et al., J Neurosci 33:4923-4934, 2013). Monomeric, oligomeric and fibrillar forms of Aβ₄₂ peptides were prepared and immobilized to the surface of biosensor chip CMS (GE Healthcare, Piscataway, N.J.) via an amine coupling of primary amino groups of the appropriate peptide to carboxyl groups in the dextran matrix of the chip. Serial dilutions of purified anti-Aβ-_{τ-τ} antibody or irrelevant IgG were injected over each immobilized form of peptide. The kinetics of binding/dissociation was measured as change of the SPR signal (in resonance units (RU)). Data were analyzed with BIAevaluation 4.1.1 software using a 1:1 interaction model to determine apparent binding constants.

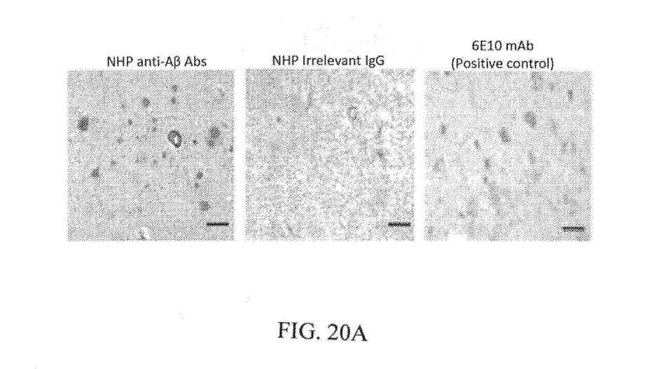

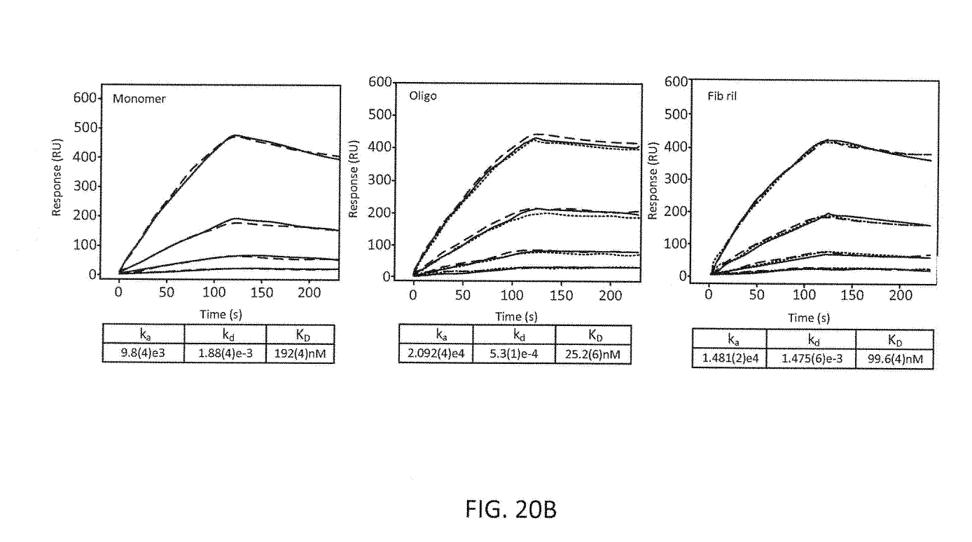

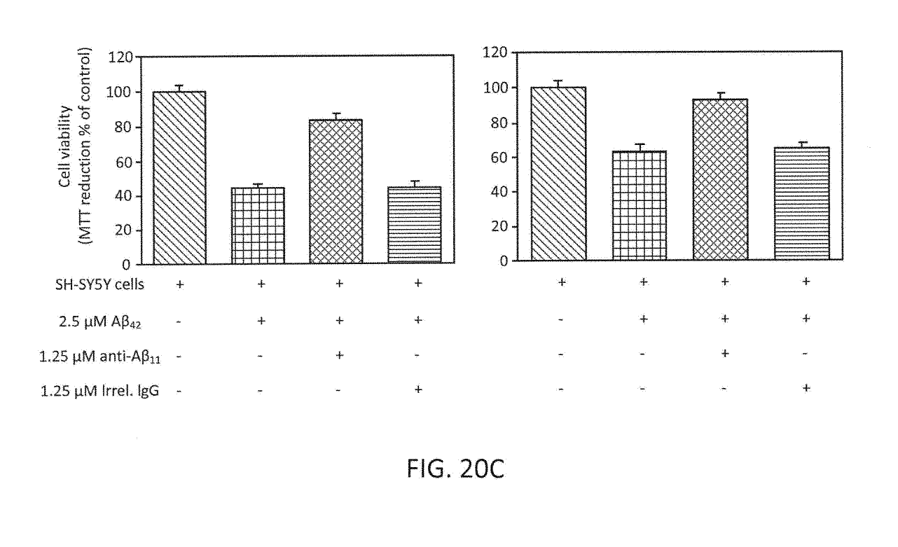

Anti-Aβ antibodies generated in different animal models (mice, rabbits and monkeys) vaccinated with MultiTEP-based AD epitope vaccines are shown to be functionally potent. Exemplary data obtained with antibodies isolated from monkey sera are presented in FIGS. 20A-20C.

Anti-Aβ antibody purified from sera of rhesus macaques vaccinated with AV-1955, but not irrelevant monkey IgG, binds to immobilized Aβ42 monomeric, oligomeric, and fibrillar forms with binding affinity 19.2×10⁻⁸, 2.5×10⁻⁸, 9.9×10⁻⁸, respectively (FIG. 20B) as measured using the Biacore. Anti-Aβ antibody but not irrelevant IgG bound to cortical plaques in brain of AD case (FIG. 20A). Furthermore, anti-Aβ antibody inhibits Aβ₄₂ fibrils- and oligomer-mediated neurotoxicity of SH-SY5Y neuroblastoma cell line (FIG. 20C). Similar results were acquired for antibodies obtained from mice and rabbits.

### Example 16

### In Vivo Therapeutic Efficacy of Antibodies Generated by MultiTep DNA Epitope Vaccine in 3×Tg-Ad Mice

In this example, the therapeutic efficacy of DNA epitope vaccine was tested in ˜4-5 mo old 3×Tg-AD mice (Oddo S; et al. Neuron 39:409-21, 2003). Vaccinated mice induced strong cellular response specific to MultiTEP component of vaccine and high production of antibodies specific to Aβ₄₂ peptide.

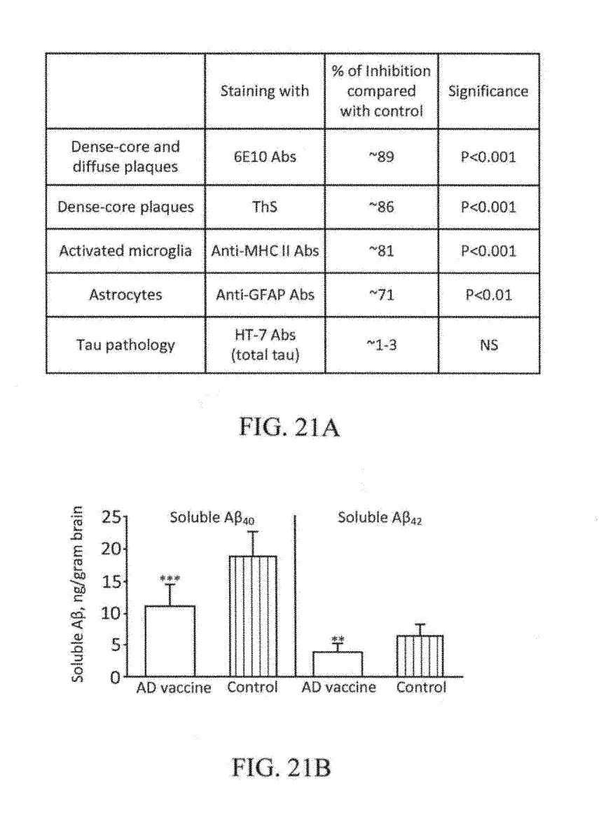

Vaccination prevented neuropathological changes in 18±0.5 mo old immune mice compared with that in control mice. Generated antibodies significantly reduced amyloid burden (diffuse and dense-core plaques) in the brains of immune mice versus control groups (FIG. 21A). Epitope vaccine induced statistically significant reduction of soluble Aβ₄₀ and Aβ₄₂ (P<0.001 and P<0.01, respectively) in the brains of immune mice (FIG. 21B). Vaccinated mice developed significantly less inflammation related pathology (microglial activation, astrocytosis) without increasing the incidence of cerebral microhemorrhages in aged 3×Tg-AD mice (FIG. 21A). The reduction of Aβ deposition was associated with less activation of astrocytosis and MHC class II positive cells. Tau pathology also showed trend toward decrease in vaccinated mice compared with that in control animals (FIG. 21A). No infiltration of T cells into the brains of mice immunized with epitope vaccine was observed.

### Example 17

### Mapping of T Cell Responses Generated by MultiTep DNA Epitope Vaccine

This example presents the mapping of immunogenic Th cell epitopes in a MultiTEP platform in mice and monkeys.

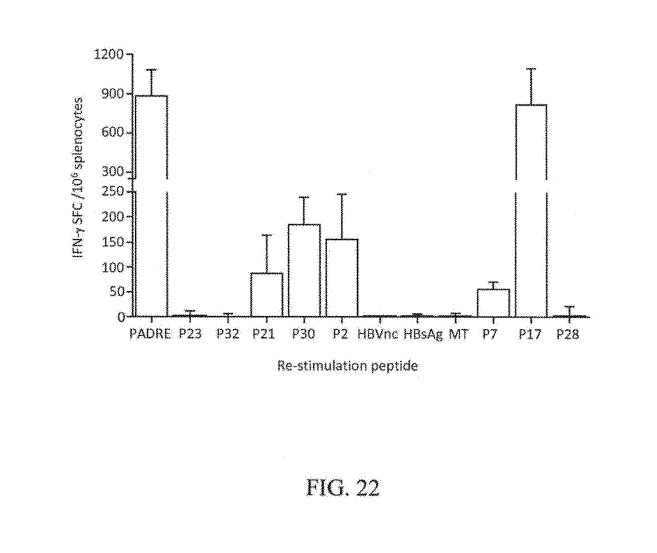

Mice of the H2-b haplotype immunized with MultiTEP based DNA epitope vaccines respond to the epitopes PADRE, P21, P30, P2, P7 and P17 (FIG. 22).

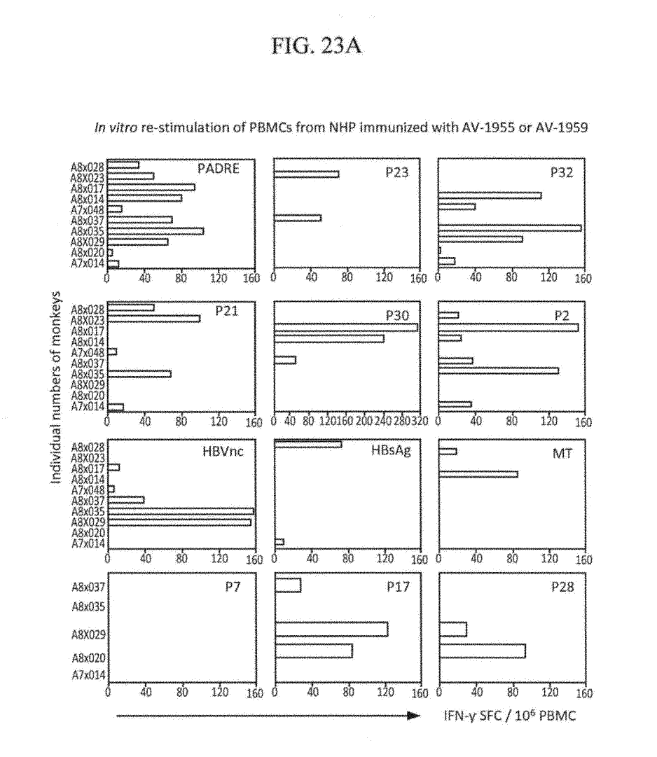

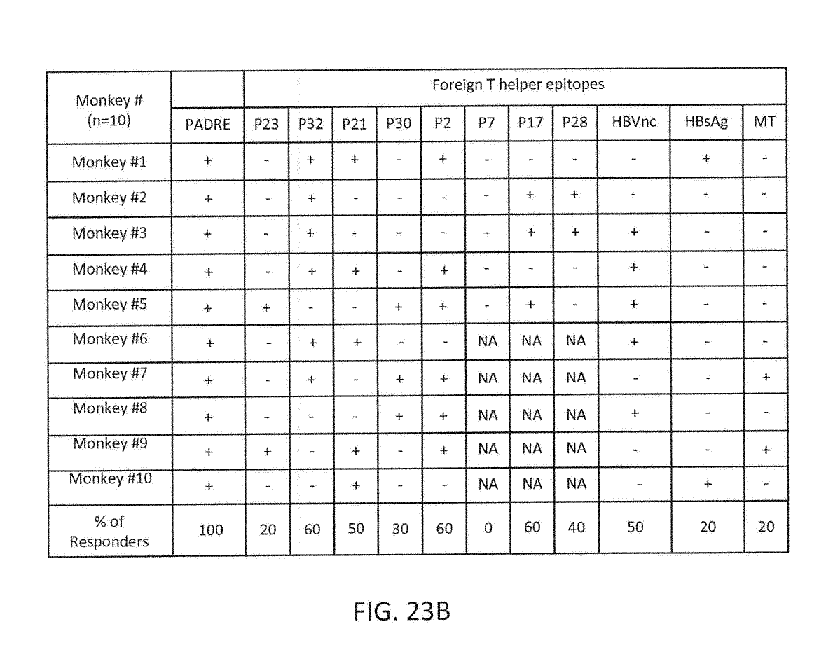

Mapping of Th cell responses in monkeys demonstrated that DNA epitope vaccine AV-1959 induced Th cell responses in all 10 macaques, although the immunogenicity of Th epitopes within the MultiTEP platform varied among individual animals. Quantitative analyses demonstrated that epitopes that are strong in one monkey, can have mediocre or weak immunogenicity in other animals. For example, strong Th cell immune responses (over 100 IFNγ positive SFC per 106 PBMC) were detected in two animals after re-stimulation of immune PBMC cultures with P32, while this response was medium (50-100 IFNγ positive SFC per 106 PBMC) in 1 macaque, weak (less than 50 IFNγ positive SFC per 106 PBMC) in 3 macaques, and no response was detected in 4 animals (FIGS. 23A-23B).

The Table in FIG. 23B presents the analyses of prevalence of Th epitopes within the NHP (non-human primate) population used in the vaccination study. The data demonstrate that each macaque with diverse MHC class II molecules responded to a different set of Th epitopes. For example, PADRE is immunogenic in 100% of macaques: PBMC from all animals responded to the re-stimulation with the synthetic promiscuous Th epitope, PADRE, which is known to be recognized by 14 of 15 human DR molecules (Alexander J, et al. Immunity 1:751-761, 1994). Next more prevalent Th epitopes are P2, P32, P17, P21 from TT and HBVnc from HBV that are immunogenic in 50-60% of vaccinated animals. The remaining Th epitopes were capable of activating Th cells in 20-30% of animals, while one Th epitope, P7 is not recognized by any of the 5 macaques immunized with AV-1959 vaccine.

### Example 18

### MultiTep Epitope Vaccine Activates Memory Th Cells Specific to Foreign Epitopes

An advantage of the epitope vaccine design is overcoming the phenomenon of immunosenescence in elderly individuals by activating pre-existing memory Th cells. In this example, we immunized mice with recombinant protein based MultiTEP epitope vaccine. Previously, the immunogenicity and the therapeutic efficacy of the first generation peptide- and recombinant protein-based vaccines in Tg2576 mice, an APP over-expressing model of AD (Hsiao K, et al. Science 1996, 274:99-102), was reported (Petrushina I, J Neurosci 2007, 27:12721-12731; Davtyan H, et al., J Neurosci 2013, 33:4923-4934).

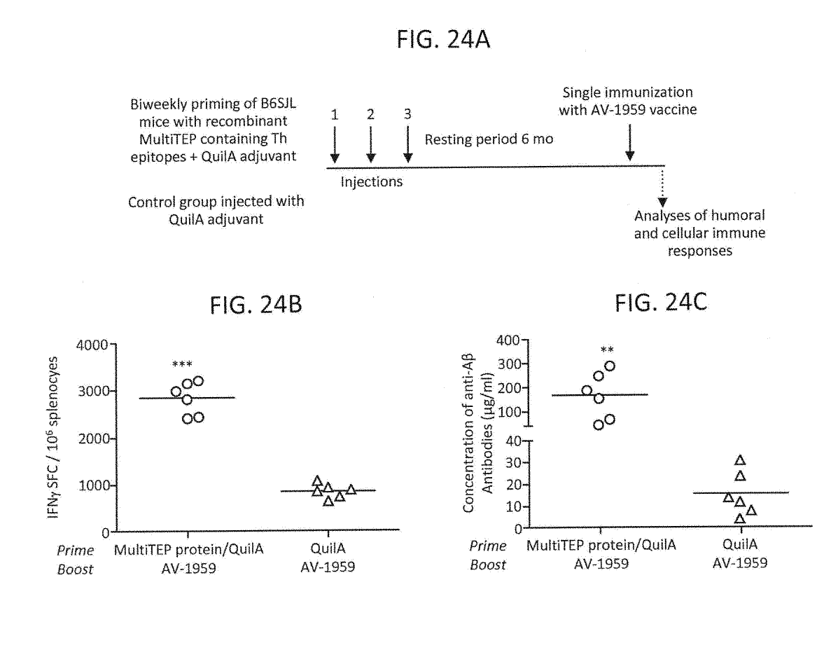

As shown herein, recombinant protein-based MultiTEP vaccine is able to induce stronger immune responses in mice possessing pre-existing memory Th cells. Two groups of B6SJL mice were immunized with recombinant protein containing only the MultiTEP component of AV-1959 vaccine formulated in QuilA, or QuilA only (FIG. 24A). After a 6-month resting period, MultiTEP-primed mice and control mice were boosted with the recombinant protein-based AV-1959 epitope vaccine and both cellular and humoral immune responses were analyzed (FIGS. 24B and 24C). Boosting of MultiTEP-primed mice with AV-1959 induced strong Th cell responses specific to MultiTEP protein: very large number of cells producing IFNγ was detected in this group of mice with pre-existing memory Th cells vs control mice (FIG. 24B). Moreover, the single injection with AV-1959 vaccine formulated in the strong Th1 adjuvant Quil A led to induction of a strong anti-Aβ antibody response only in mice with pre-existing memory Th cells: concentrations of anti-Aβ antibodies were significantly higher (P<0.001) than that in control mice (FIG. 24C). These results demonstrate that even a single immunization with epitope vaccine strongly activated pre-existing memory CD4+ T cells specific to the Th epitopes of this vaccine and rapidly led to the robust production of antibodies specific to the B cell epitope of the same vaccine.

Activation of pre-existing memory T cells and rapid production of high concentrations of anti-Aβ antibodies had a therapeutic effect and led to delay of cognitive impairment and the accumulation of pathological Aβ in Tg2576 mice.

Two groups of 5 mo old mice were injected with either MultiTEP protein formulated in QuilA or QuilA only (control) 3 times bi-weekly. Six months after the last injection, at the age of 11 mos, mice were boosted monthly with protein-based AV-1959 epitope vaccine formulated in QuilA until they reached the age of 16 mos. Control mice were injected with QuilA only. After a single boost with epitope vaccine, a strong anti-Aβ antibody response was detected in mice with pre-existing memory Th cells. Concentrations of anti-Aβ antibodies in these mice were significantly higher (P<0.001) than that in mice primed with QuilA only, and boosted with vaccine (32.20±10.55 μg/mL vs 0.82±0.24 μg/mL, respectively). After boosts the antibody responses reached to the equal level in both groups (data not shown).

The effect of vaccination on delay of cognitive impairment in mice was tested by “Novel Object Recognition” test. Each mouse was habituated to an empty arena for 5 min one day prior to testing. On the first day of testing, mice were exposed to two identical objects placed at opposite ends of the arena for 5 minutes. Twenty-four hours later, the mouse was returned to the arena, this time with one familiar object and one novel object. Time spent exploring the objects was recorded for 5 minutes. The recognition index represents the percentage of the time that mice spend exploring the novel object. Objects used in this task were carefully selected to prevent preference or phobic behavior. Although both experimental groups showed improved behavior, only mice with pre-existing memory T cells achieved a recognition index significantly higher than control mice (data not shown). Thus, although mice from both groups had an equal level of antibodies at the time of behavior testing, more rapid generation of high concentrations of anti-Aβ antibodies in mice with pre-existing memory T cells at the start of boosting was more beneficial to the mice. The improvement in cognitive function was associated with less profound neuropathological changes in brains of mice with pre-existing memory Th cells compared with both control non-immunized mice or mice without pre-existing memory Th cells at the time of boosting injection.

### Example 19

### Epitope Vaccine Targeting Alpha-Synuclein

This example demonstrates that an α-syn-based epitope vaccine induces strong anti-αsyn antibody response without generating cellular immune responses specific to this self molecule.

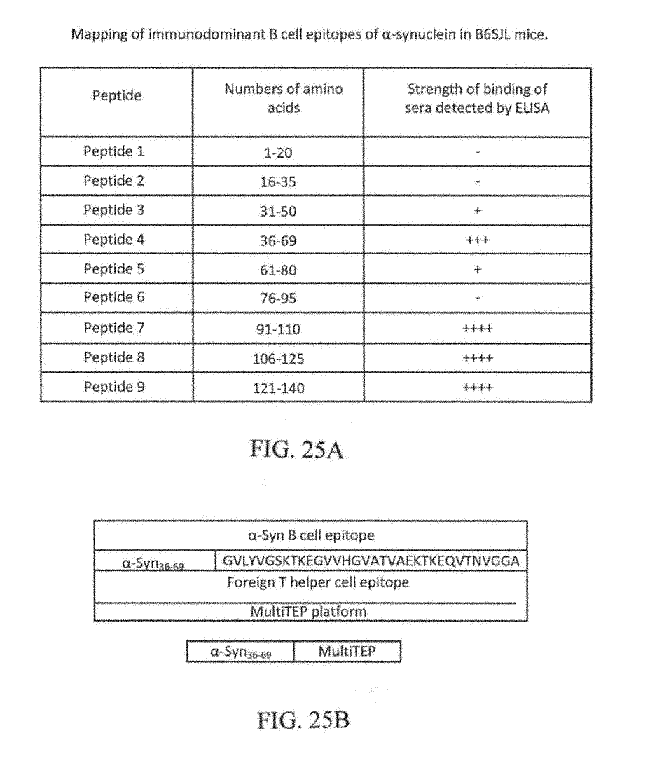

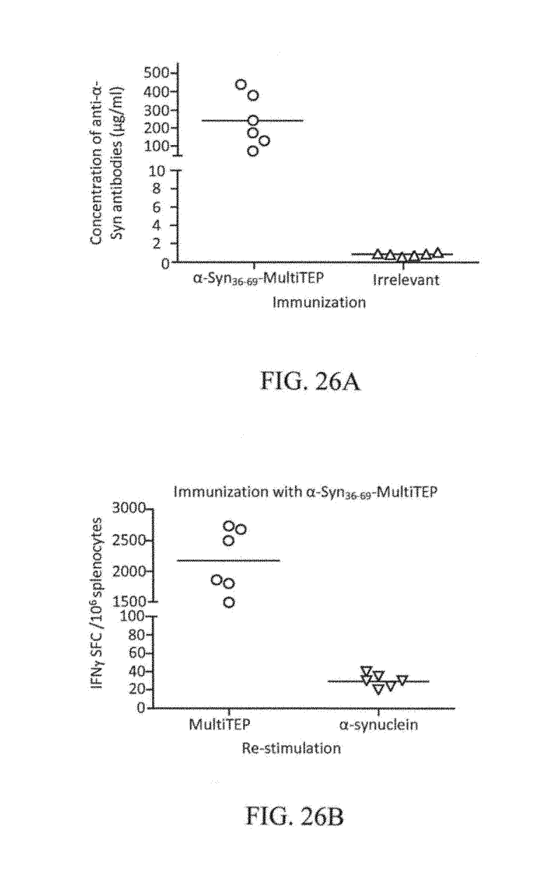

To identify immunodominant B cell epitopes of α-synuclein, mice were immunized with DNA encoding full-length α-synuclein fused with promiscuous strong Th cell epitope PADRE. Sera from vaccinated mice, collected after the third immunization were used for mapping of B-cell epitopes using 9 overlapping 20-mer peptides constituting α-syn protein. Antibodies specific to six different peptides were detected (FIG. 25A). Three of six B-cell epitopes that are localized at the C-end region of α-syn coincide with the epitopes previously detected (Masliah E, et al. Neuron 46:857-868, 2005). Selected peptides were tested for whether they possess a Th cell epitope (data not shown). Epitope 36-69 was selected for generation of epitope vaccine. Recombinant protein composed of α-syn₃₆₋₆₉ attached to MultiTEP platform (FIG. 25B) purified from E. coli. B6SJL mice were immunized with this immunogen formulated in QuilA adjuvant. Both B and T cell responses were analyzed after three bi-weekly immunizations. Control animals were injected with adjuvant only. α-syn₃₆₋₆₉-MultiTEP induced strong antibody responses specific to the appropriate peptide (data not shown) and full-length human α-syn (FIG. 26A). Cellular immune responses were measured by ELISPOT (FIG. 26B). Mice immunized with α-syn₃₆₋₆₉-MultiTEP induced robust T cell responses after re-stimulation with MultiTEP protein, but not with full-length α-synuclein protein (FIG. 26B) or α-syn₃₆₋₆₉ peptide (data not shown). Thus, it was confirmed in mice of the H2bxs haplotype that α-syn₃₆₋₆₉ does not possess a T cell epitope.

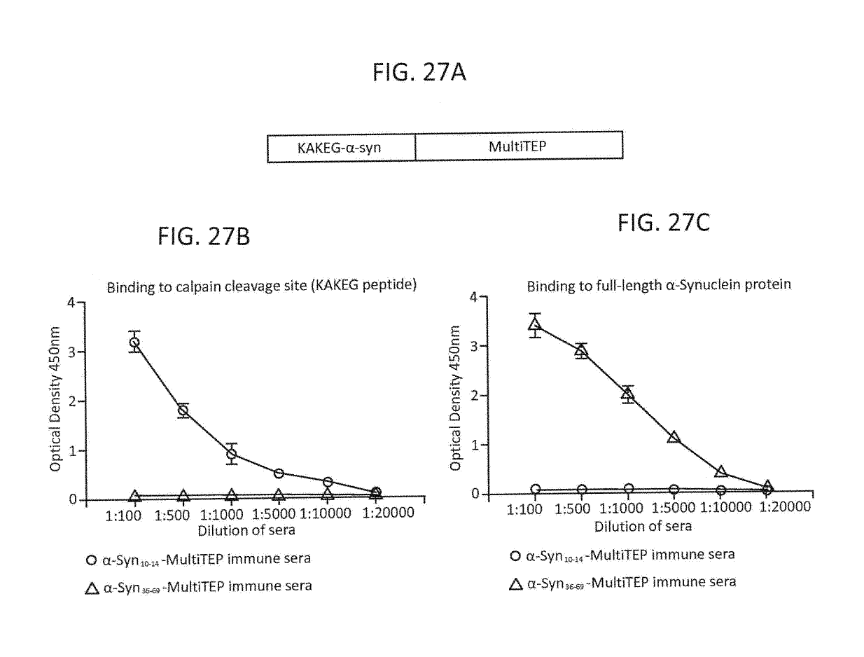

Recently, it was shown that calpain I cleaves the pathological form of α-syn generating a unique α-syn fragment. This α-syn fragment has an N-terminal sequence KAKEG (aa 10-14). KAKEG was tested as a B-cell epitope, a novel immunotherapy target for generation of antibodies inhibiting aberrant accumulation of α-syn in the central nervous system. A DNA vaccine encoding KAKEG fused to MultiTEP platform was generated and C57BI/6 mice were immunized using gene gun (biweekly, 3 times). Vaccinated mice generated strong antibody responses to KAKEG (FIG. 27A). In addition, this vaccine did not induce antibodies specific to full length α-syn, while this human protein was recognized by immune sera (positive control) collected from mice immunized with α-syn₃₆₋₆₉-MultiTEP (FIG. 27B).

Immune sera from vaccinated mice was tested for recognition of pathological forms of α-syn in the human brain from the DLB case by IHC or IP/WB. Antibodies generated after immunizations with both α-syn₃₆₋₆₉-MultiTEP and KAKEG-MultiTEP, which did not recognize full length α-syn, showed positive staining of brain sections, an indication that these antibodies recognized the pathological form of α-syn. Control brain sections showed negative staining.

These experiments evidence that (i) epitope vaccine based on α-syn₃₆₋₆₉ fused with foreign Th cell epitopes (MultiTEP platform) induced high titers of anti-α-syn antibody; (ii) antibodies generated by epitope vaccine are functional, since they bind to native α-syn ex vivo (iii) peptide α-syn₃₆₋₆₉ did not contain autoreactive Th cell epitopes, and hence can be used in an epitope vaccine; (iv) KAKEG-MultiTEP epitope vaccine induced strong antibody responses specific to KAKEG, but not to full length α-syn; and (v) antibodies specific to the KAKEG neoepitope recognized pathological form of α-syn and could also be used for the generation of a DNA epitope vaccine.

### Example 20

### Epitope Vaccine Targeting Pathological Tau Protein

This example describes the selection of tau epitope and generation and testing of an epitope vaccine targeting pathological tau.

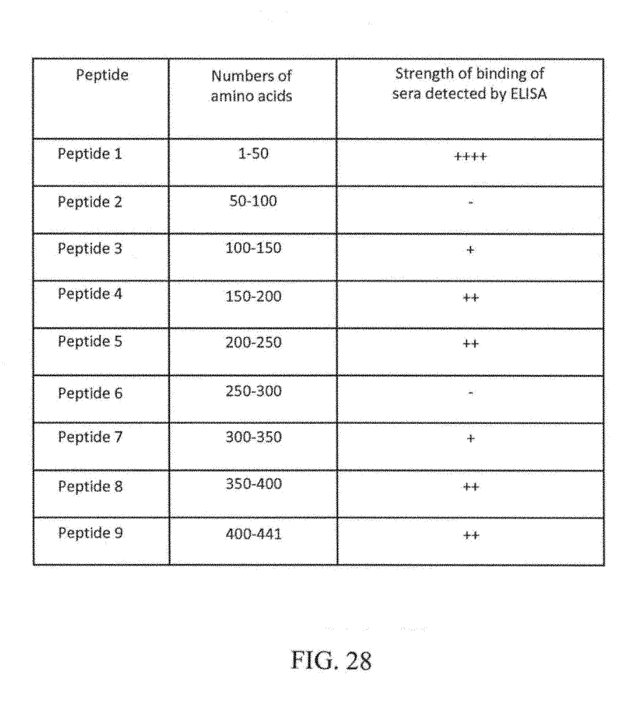

Mapping of tau B cell epitopes. To map potentially important non-phosphorylated tau regions for the generation of therapeutic antibodies, anti-sera were obtained from tau transgenic mice rTg4510 (transgene is a human 4-repeat tau carrying P301L mutation controlled by cytomegalovirus minimal promoter and upstream tetracycline operator (tetO)) immunized with full length of tau (N2/4R). ELISA was used to detect binding of polyclonal sera to recombinant tau proteins from 1 aa to 50 aa, from 50 aa to 100aa, from 100aa to 150aa; from 150aa to 200aa, from 200aa to 250aa; from 250aa to 300aa; from 300aa to 350aa; from 350aa to 400aa; from 400aa to 441 aa; thus we checked entire sequence of N2/4R molecule. Data demonstrated that anti-tau antibodies bind strongly to regions spanning aa 1 to 50 of tau protein and do not bind aa 50-100 or 250-300 (FIG. 28). Moderate binding was detected in wells coated with recombinant tau proteins spanning aa 150 to 200, 200 to 250; 350 to 400; and 400-441. Finally low binding was detected in wells coated with recombinant tau proteins spanning aa 100 to 150 and 300-350. These data provided the basis for selecting epitopes for generation of tau-targeting epitope vaccines important for active immunotherapy of subjects with taupathy. Tau region comprising 2-18 aa was selected for generation of epitope vaccine.

The aa2-18 region of tau is normally hidden due to folding of the protein, and it is exposed during aggregation of tau (Morfini G A, et al. J Neurosci 2009, 29:12776-12786; Horowitz P M, et al. J Neurosci 2004, 24:7895-7902). The aa2-18 region, also termed phosphatase-activating domain (PAD), plays a role in activation of a signaling cascade involving protein phosphatase I and glycogen synthase kinase 3, which leads to anterograde FAT inhibition. The exposure of PAD that is required for inhibition of FAT may be regulated by phosphorylation of PAD, as well as by N-terminal truncation of tau protein that occurs during formation of NFT. Phosphorylation of Y18 as well as truncation of N-terminal region of tau may remove a toxic region and have a protective role. Therefore, antibodies generated against this epitope may recognize pathologic, but not normal Tau. In such a case, the epitope vaccine may induce antibodies that will target very early stages of tauopathy.

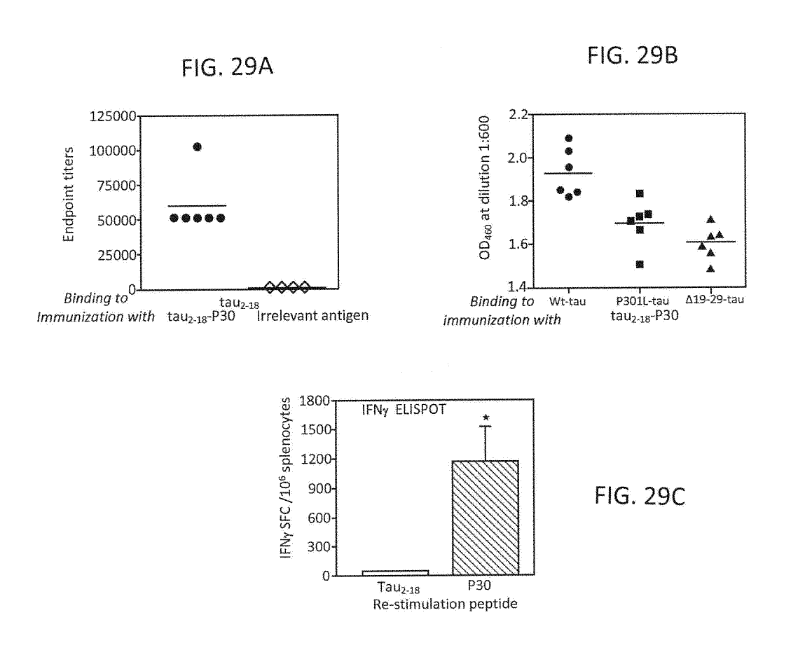

To generate the epitope vaccine, tau₂₋₁₈ epitope was fused with a foreign promiscuous Th epitope of TT (P30). B6SJL mice of H2bxs haplotype were immunized with a tau₂₋₁₈-P30 vaccine formulated in a strong Th1 adjuvant Quil A (the same as QS21). Both humoral (ELISA) and cellular (ELISPOT) immune responses were measured. Immunization induced high titers of tau₂₋₁₈-specific antibodies (FIG. 29A) that also recognized 4R/0N wild/type Tau, 4R/0N P301 L Tau, and 4R/0N Tau with deleted region 19-29aa in ELISA (FIG. 29B). The epitope vaccine also induced a strong T cell response that was specific to P30, but not to tau₂₋₁₈ (FIG. 29C). Thus, the tau₂₋₁₈-P30 vaccine formulated in QuilA adjuvant did not activate autoreactive Th cells while it generated strong non-self cellular immune responses and production of antibodies specific to various Tau proteins.

### Example 21

### Anti-Tau Antibodies Bind to Pathological Tau in Brains from AD Case

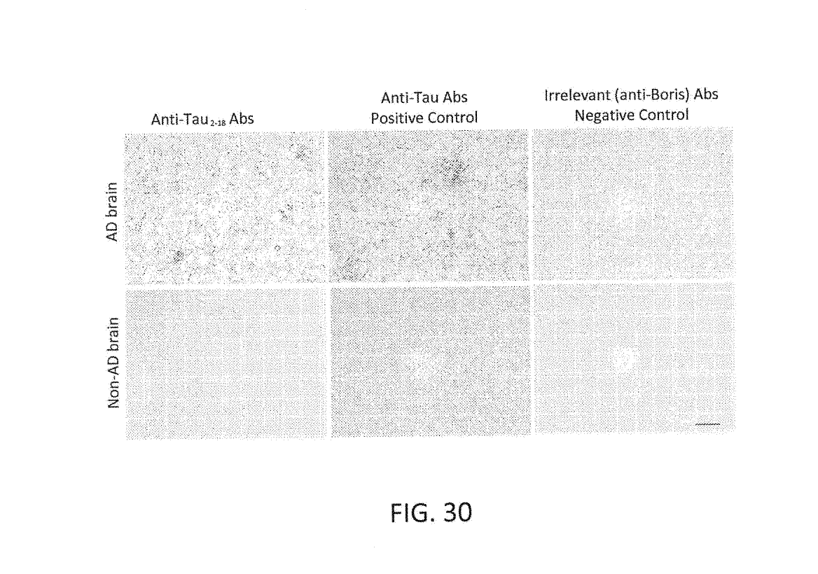

In this example we demonstrate the ability of anti-tau antibodies to bind pathological tau in brain sections from AD case. Sera from experimental mice immunized with the epitope vaccine and control animals immunized with irrelevant antigen were assayed on brain sections from AD and non-AD cases. Results showed that immune sera from experimental, but not control, mice at dilution 1:500 recognized NFT in the brain from AD case (Tangle stage V, Plaque stage C; FIG. 30). The same immune sera did not bind normal tau in a non-AD case. Therefore, tau epitope vaccine induced antibody responses specific to the pathological form of tau.

### Example 22

### Antibodies Block the Cell-Cell Propagation of Tau Aggregates

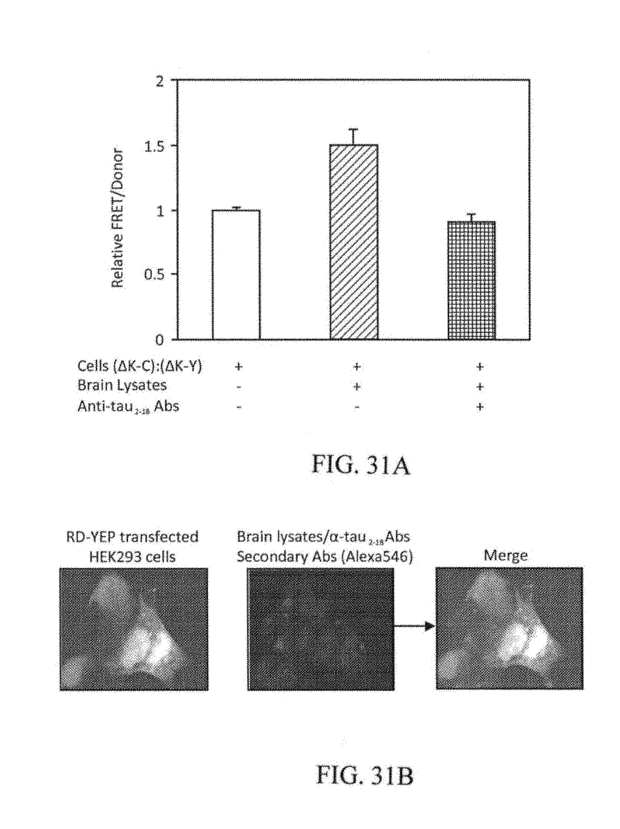

In this example, we demonstrate the therapeutic potential of anti-tau antibodies to block full-length tau aggregates from entering a cell and inducing aggregation of intracellular tau repeat domain (RD), the aggregation-prone core of Tau protein with mutation at position 280 (ΔK280) [RD(ΔK)] (Kfoury N, et al. J Biol Chem, 287:19440-19451, 2012). More specifically, a fluorescence resonance energy transfer (FRET) assay has been used for tracking the aggregation of the RD(ΔK)-CFP and RD(ΔK)-YFP proteins in HEK293 cells co-transfected with constructs expressing mentioned proteins that referred to (ΔK-C):(ΔK-Y) in FIGS. 31A-31B. The more vigorous aggregation that was induced by adding brain lysate of P301S Tg mice containing full-length Tau aggregates to the culture of co-transfected cells increased FRET signal. Pre-treatment of brain-lysate with anti-tau₂₋₁₈ antibody trapped the tau aggregates on a surface of cells, inhibiting induction of (ΔK-C):(ΔK-Y) aggregation and decreased FRET signal to baseline level (FIG. 31A). In addition, using confocal microscopy, brain lysate/anti-tau₂₋₁₈ antibody complexes are shown to internalize into the RD-YFP transfected cells (FIG. 31B). Antibodies were not detected in non-transfected (NT) cells or in YFP cells in the absence of tau aggregates (data not shown). When RD(ΔK) was replaced with a mutant form of tau containing two proline substitutions, I227P and I308P (termed PP), which inhibit β-sheet formation and fibrillization, no internalization of antibodies was observed (data not shown).

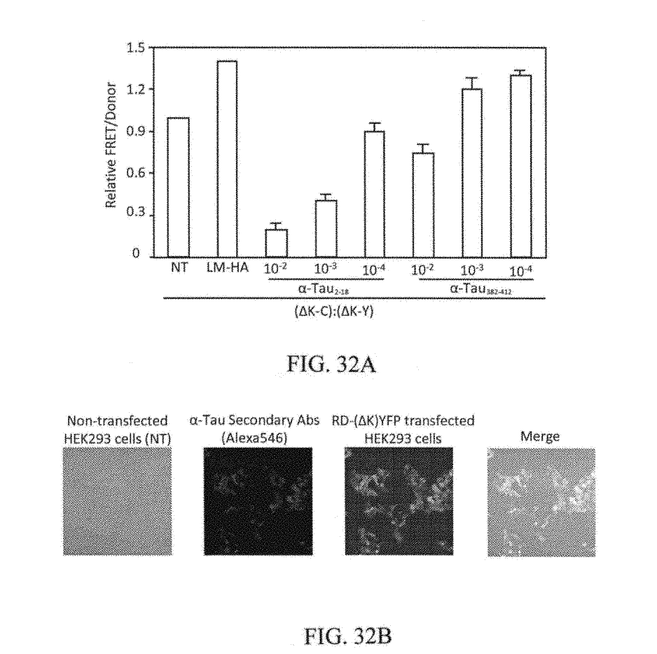

In another set of experiments the ability of anti-tau₂₋₁₈ antibodies to block trans-cellular movement of aggregated tau was tested. HEK293 cells were transfected with construct expressing hemagglutinin-tagged tau (RD) containing two disease-associated mutations that increase the capacity of protein to aggregate: P301L and V337M (LM) (LM-HA). When these cell populations were co-cultured with HEK293 cells expressing RD(ΔK)-CFP and RD(ΔK)-YFP proteins, trans-cellular propagation of LM-HA aggregates from donor cells (HEK293 cells transfected with LM-HA) induces aggregation of ΔK-C:ΔK-Y in recipient cells (HEK293 transfected with RD-CFP/RD-YFP) as detected by FRET between CFP and YFP. If anti-tau antibodies are added to this system and block propagation of tau, then FRET signal is decreased. Two antibodies specific to tau₂₋₁₈ and Tau₃₈₂₋₄₁₂ (generated in rats by immunization with Tau₃₈₂₋₄₁₂-PADRE) added to culture media at the indicated dilutions (10⁻², 10⁻³ and 10⁻⁴) during the 48 h co-culture period inhibited the cell-cell propagation of tau aggregates. Relative FRET across each group tested is shown in FIG. 32A. In addition, using confocal microscopy anti-tau antibodies are demonstrated to bind RD-YFP aggregates on a surface of transfected HEK293 cells (FIG. 32B).

These data suggest that α-tau₂₋₁₈ and α-tau₃₈₂₋₄₁₂ antibodies recognize a conformational antigenic determinant (mimotope/s) in aggregated RD. In addition, therapeutic anti-tau antibodies can be generated without using phosphorylated tau molecules or their derivatives (e.g., B cell epitopes) as an immunogen. Instead non-phosphorylated tau could be used for generation of therapeutic antibodies that will be safe to administrate to subjects with tauopathy, because such antibodies will not get inside the cells and inhibit function of normal tau molecules.

### Example 23

### Generation and Testing of Multivalent DNA Epitope Vaccine

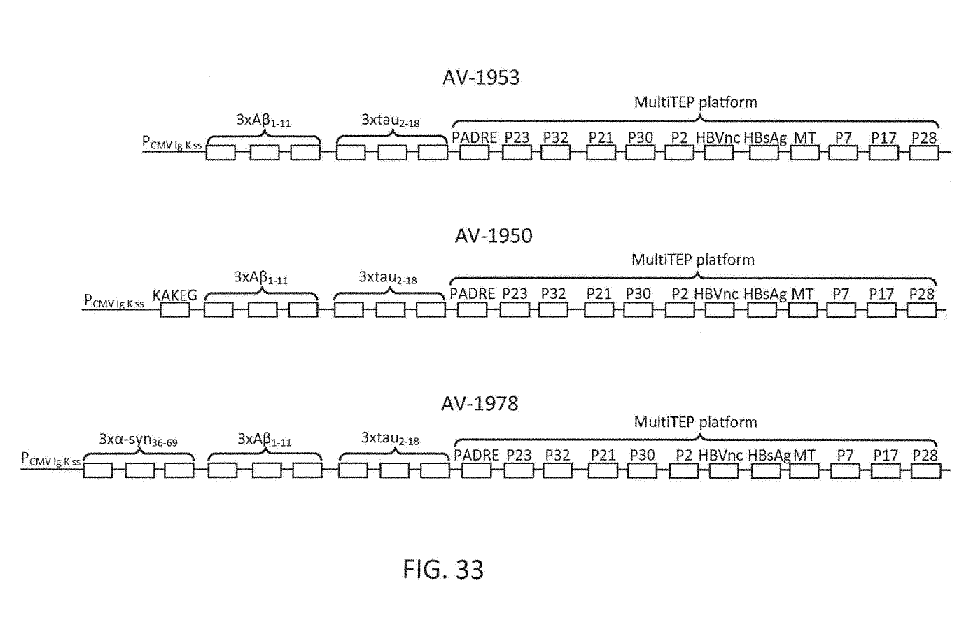

In this example, DNA epitope vaccines are generated that contain different combinations of B cell epitopes (FIG. 33) and tested. The vaccines generated contain (i) three copies of Aβ B cell epitope comprising aa 1-11 and three copies of Tau B cell epitope comprising aa 2-18; (ii) three copies of B cell epitope of α-syn comprising aa 36-69, three copies of Tau epitope comprising aa 2-18, and three copies of Aβ epitope comprising aa 1-11; and (iii) KAKEG epitope of α-syn, three copies of Tau epitope comprising aa 2-18, and three copies of Aβ epitope comprising aa 1-11. In all constructs B cell epitopes were fused to a string of foreign T cell epitopes. Each copy of B cell epitope and T cell epitope was separated by a GS small linker sequence (FIG. 33). The expression of the immunogen from plasmids containing these constructs was demonstrated using transiently transfected CHO cells (data not shown).

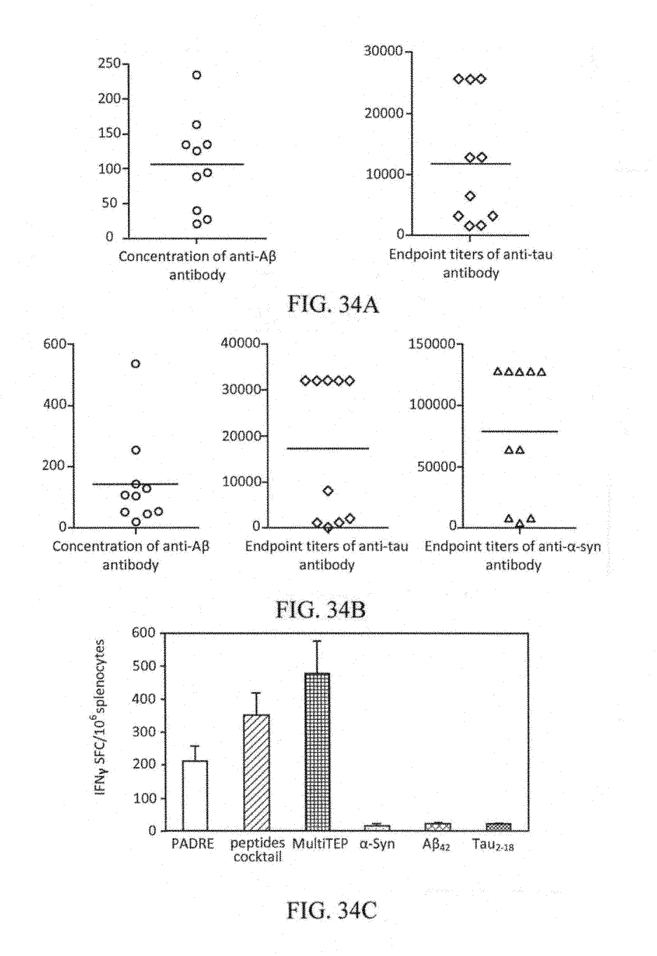

The DNA epitope vaccines were used for immunization of B6SJL mice (6 per group, 3 monthly injections) of H2bxs immune haplotype. Control animals were injected with an irrelevant DNA vaccine. Mice vaccinated with bivalent epitope vaccine (AV-1953) generated strong antibody responses to Aβ₄₂ and Tau protein (FIG. 34A). Mice vaccinated with trivalent epitope vaccines (AV-1950 and AV-1978) generated strong antibody responses to α-syn, Aβ₄₂ and Tau protein (FIG. 34B). Cellular immune responses were also measured and demonstrated that mice immunized with multivalent epitope vaccines induced robust T cell responses after re-stimulation with recombinant protein MultiTEP or a mix of peptides representing Th epitopes in a construct (FIG. 34C), but not with the α-syn, Tau, or Aβ₄₀.

### Example 24

### Selection of an Optimal Adjuvant for Anti-Aβ Vaccine

To determine whether delta inulin-based adjuvants are superior to other adjuvants that are approved by FDA or used in clinical trials, we tested the ability of commercial adjuvants Alhydrogel®, Montanide-ISA5I, Montanide-ISA720, and MPLA-SM along with Advax™ and Advax^{CPG} to enhance the antibody response to recombinant protein based vaccine AV-1959R providing lowest variability of antibody levels. Quil-A, a less purified version of QS21, the adjuvant that was used in the AN-1792 clinical trial, was used in parallel as a control adjuvant for mice. AV-1959R is composed of three copies of Aβ B cell epitopes fused with MultiTEP platform composed of synthetic universal Th epitope PADRE and eleven foreign Th epitopes from tetanus toxoid, HBV and flu.

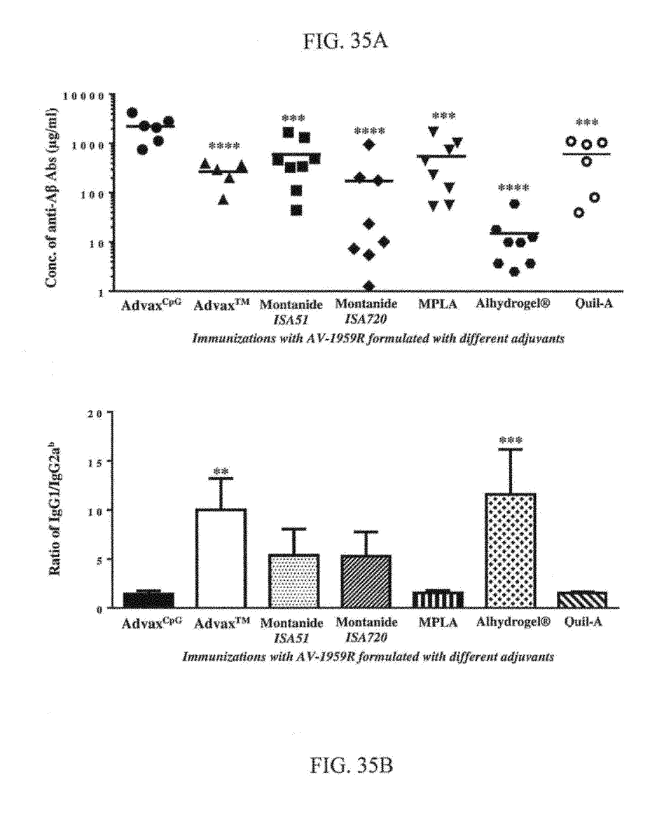

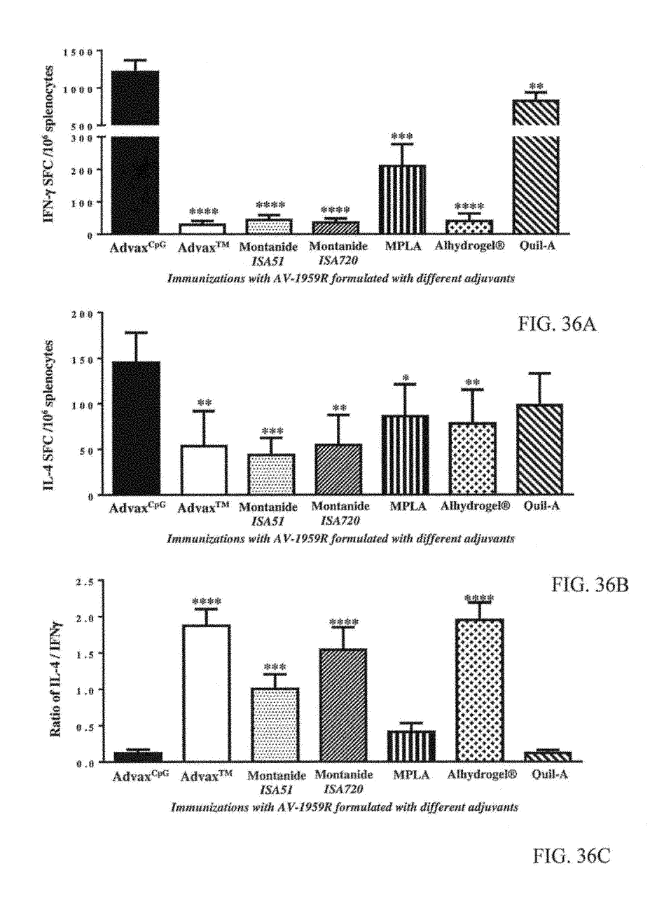

The results showed that AV-1959R formulated with Advax^{CpG} induced significantly stronger antibody responses than all the other adjuvants with a low variability in responses between animals in the Advax^{CpG} group (FIG. 35A). Analysis of antibody isotypes specific for Aβ showed that Alhydrogel®, Advax™, Montanide-ISA51 and -ISA720 adjuvants induced primarily an IgG1 (Th2) response, whereas Advax^{CpG} and MPLA shifted the response toward IgG2a^{b}, a Th1 response associated isotype (FIG. 35B). To further explore adjuvant effects on Th1 and Th2 phenotype, we measured the numbers of splenocytes producing IFN-γ and IL-4 cytokines by ELISpot (spot-forming cells, SFC) and found that the Advax^{CpG} group produced significantly higher frequencies of IFN-γ⁺ and IL-4⁺ Th cells than all other GMP adjuvant groups (FIGS. 36A and 36B). The TLR4 agonist, MPLA was the only other GMP-grade adjuvant that generated significant numbers of both IFN-γ⁺ and IL-4⁺ Th cells, although these were approximately 5 and 1.5 times, respectively, lower than those induced with Advax^{CpG} (FIGS. 36A and 36B). The level of Th1 responses induced by the control adjuvant, Quil-A, were comparable to MPLA, but significantly lower than Advax^{CpG}. Calculation of the ratio of IL-4/IFN-γ positive Th cells (FIG. 36C) supported the antibody isotypes data and confirmed that Advax^{CpG} was the strongest combined Th1 and Th2 adjuvant followed by MPLA, while other adjuvants only generated primarily Th2 responses to immunizations with AV-1959R. Finally, Advax^{CpG} was also well tolerated by all animals with no evidence of either local or systemic vaccine adverse reactions.

### Example 25

### Immunogenic Efficacy of Different AD Vaccines Targeting Aβ and Tau Formulated with Advax^{CPG} Adjuvant in Wildtype Mice

To determine whether the Advax^{CpG} enhances the antibody responses to different antigens equally well, three groups of C57BL6 mice were immunized with AV-1959R, AV-1980R, AV1953R and mixture of two proteins (AV-1959R+AV-1980R) formulated in Advax^{CpG}.

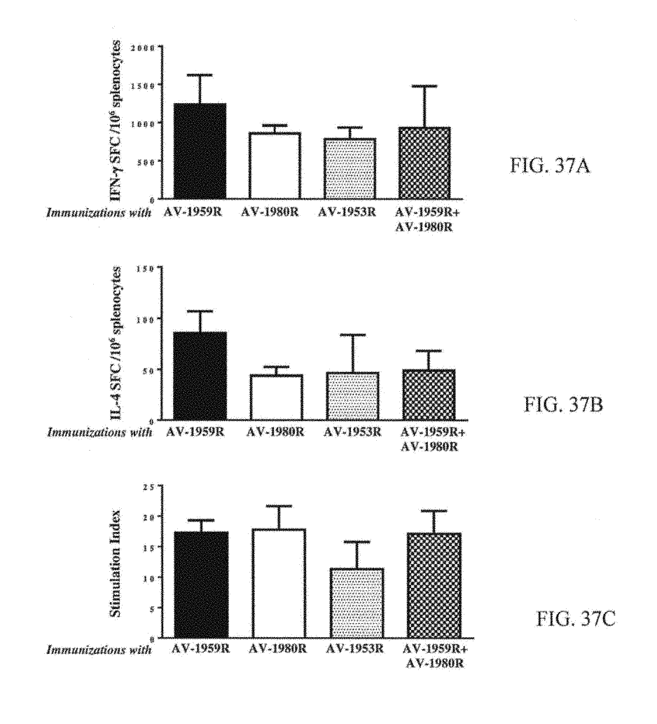

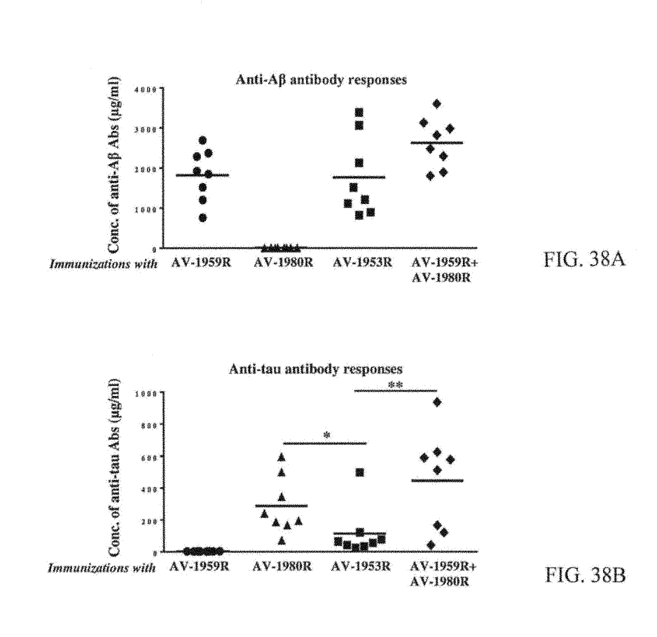

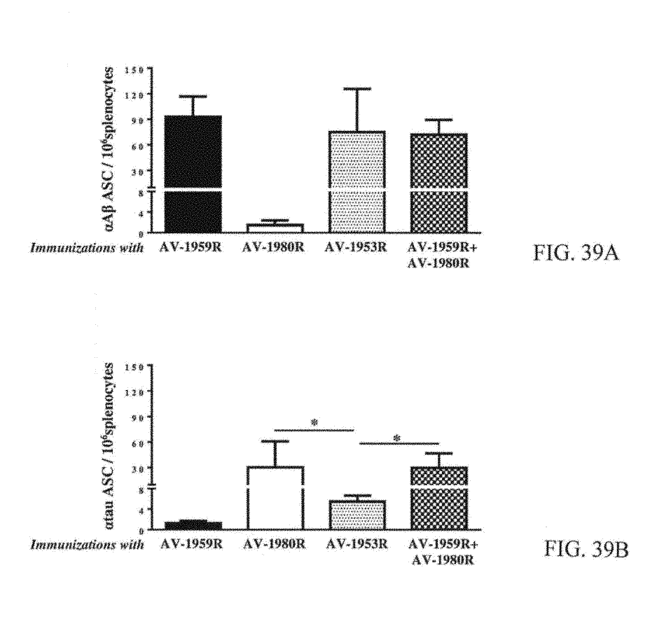

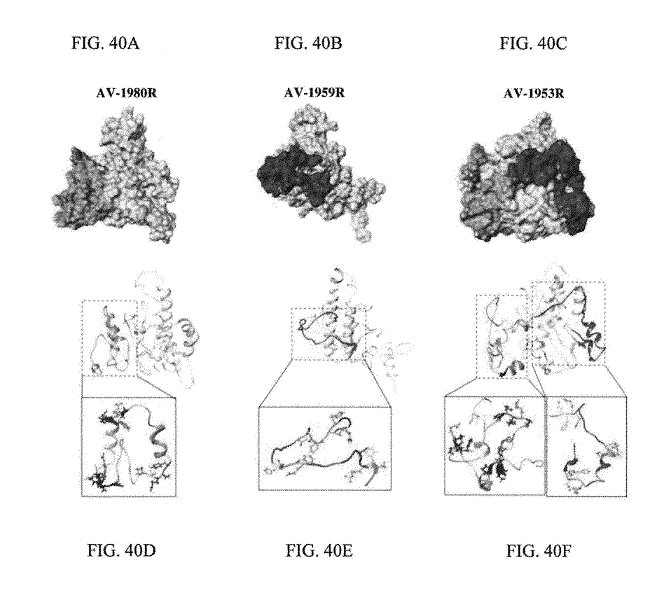

All tested AD vaccines formulated with Advax^{CpG} adjuvant generated equally strong T cell responses, measured by detection of IFN-γ⁺, IL4⁺ SFC or splenocytes proliferation specific to foreign Th cell epitopes incorporated in the MultiTEP platform (FIGS. 37A-37C). Generation of strong cellular immune responses to Th epitopes supported the production of equally high concentrations of anti-Aβ antibodies in mice vaccinated with AV1959R+AV-1980R combination, AV-1959R, or AV-1953R (FIG. 38A). As expected, immunization with AV-1980R did not generate anti-Aβ antibodies. It should be mentioned that concentrations of anti-tau antibodies were significantly lower in mice immunized with AV-1953R compared to mice vaccinated with the AV-1959R+AV-1980R combination or AV-1980-R alone (FIG. 38B). These antibody response patterns were mirrored by the frequency of antibody secreting B cells (ASC); numbers of anti-Aβ ASC were similar in mice immunized with single or combined vaccine formulations while the numbers of anti-tau ASC were significantly lower in mice vaccinated with the dual-epitope AV-1953R vaccine (FIGS. 39A and 39B). These differences could be associated with different presentation of tau B cell epitopes attached to MultiTEP on the surface of the dual-epitope AV-1953R vaccine compared with the single epitope constructs. To address this possibility, in silico structural modeling and analyses of the MultiTEP platform-based AV-1980R, AV-1959R and AV-1953R vaccines have been performed (FIGS. 40A-40F). Data suggested that on AV-1980R, two of the three tau epitopes are linear with the side chains of the critical amino acid residues accessible on the surface (FIGS. 40 A and 40D), while in AV-1959R, all three Aβ epitopes are linear with the side chains of the critical amino acid residues accessible on the surface (FIGS. 40 B and 40E). On AV-1953R, two out of three Aβ and two out of three tau epitopes are linear, however, only side chains of Aβ, but not critical tau amino acid residues are easily accessible (FIGS. 40 C and 40F). Hence, changes in the epitope structure in combination with alterations in the side chain accessibility of critical residues in the epitopes may have led to the reduced anti-tau immunogenicity of the AV-1953R dual epitope construct.

### Immune Sera Recognize Various Pathological Forms of Aβ and Tau Molecules in AD Brains

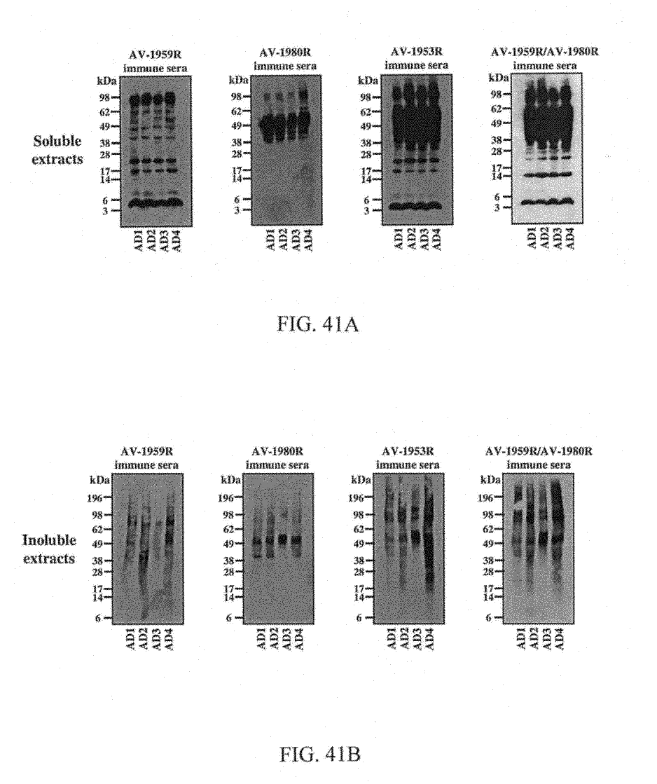

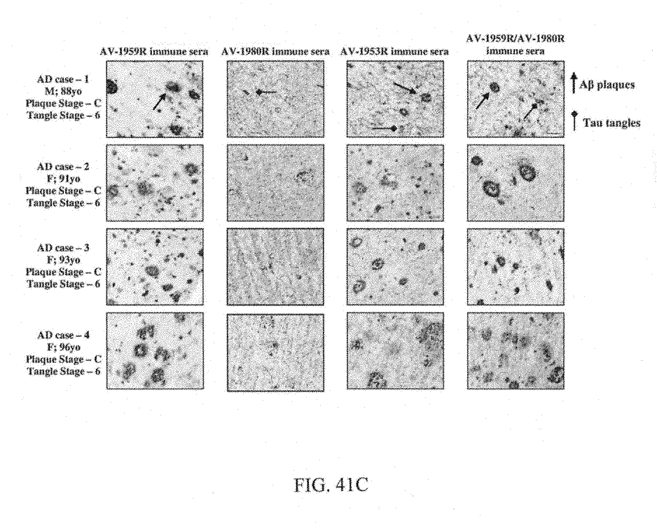

To demonstrate the effectiveness of antibodies generated in mice immunized with single vaccines, AV-1959R or AV-1980R, the mixture of two vaccines (AV-1959R/AV-1980R) or dual vaccine (AV-1953R), we analyzed the binding of immune sera to various pathological forms of Aβ and Tau in brain tissues from four different AD cases by Western Blot (WB) (FIGS. 41A and 41B) and immunohistochemistry (IHC) (FIG. 41C). The AV-1959R-immune sera bound monomeric Aβ in soluble as well as low and high molecular weight oligomers in both soluble and insoluble fractions of brain homogenates. As expected, AV-1980R-immune sera recognized monomeric tau as well as multiple larger and smaller species of tau in both soluble and insoluble fractions of brain homogenates. What is more important, antibodies generated by either the mixture of vaccines (AV-1959R/AV-1980R) or the dual vaccine (AV-1953R) recognized the same species of Aβ and tau that were detected by antisera isolated from mice vaccinated with appropriate single vaccines (AV-1959R and AV-1980R). Similar results have been obtained by IHC analyses of the same brain tissues (FIG. 41C). AV-1959R-immune sera bound senile plaques only, AV-1980R-immune sera bound NFTs and neuritic threads, yet sera from mice immunized with AV-1959R/AV-1980R mixture or AV-1953R bound both pathologies: plaques, neuritic threads and NFTs. Therefore, both mixture of MultiTEP-platform based vaccines and the dual vaccine could be an effective active immunotherapeutic strategy for targeting both misfolded proteins involved in AD pathology.

### Cross Synergism in MultiTEP-Based Vaccines Targeting Different Antigens

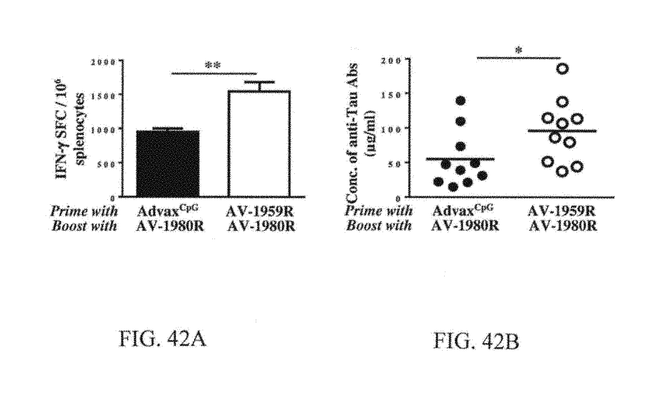

Universal MultiTEP vaccine platform is based on a string of Th foreign epitopes which, as shown in monkeys, can stimulate immune responses in a broad population of subjects with high MHC class II gene polymorphisms10. Moreover, the universal MultiTEP platform may allow using two vaccines targeting Aβ and tau at early and late stages of the disease, respectively. At the initiation of anti-tau immunotherapy AD patient immunized previously with anti-Aβ vaccine would have large numbers of MultiTEP-specific memory Th cells and hence will rapidly generate therapeutic concentrations of antibodies. To simulate this situation, mice were immunized with AV-1959R formulated in AdvaxCpG or injected with AdvaxCpG only (control) and both groups were boosted with vaccine targeting tau B cell epitope, AV-1980R. Boosting of AV-1959R vaccinated mice with AV-1980R, but not sham-injected mice, induced significantly higher cellular (FIG. 42A) and humoral (FIG. 42B) immune responses, thus proving the synergistic effect of sequential immunization with different MultiTEP vaccines.

### Example 26

### Testing the Immunogenicity and Therapeutic Efficacy of Tau AV-1980R Vaccine Formulated in Advax^{CPG} in PS19 Mouse Model of Tauopathy

To determine the potency of Advax^{CpG} to enhance antibody responses in different mouse strains including transgenic mice, in this example we tested the efficacy of AV-1980R vaccine formulated in Advax^{CpG} in PS19 mouse model of tauopathy expressing the 383 aa isoform of human tau with the P301S mutation under the control of the murine Thy1 promoter. 1.5-2 mo old PS19 mice were immunized with AV-1980R formulated in Advax^{CpG} adjuvant and antibody responses were evaluated after 2, 3 and 4 immunizations.

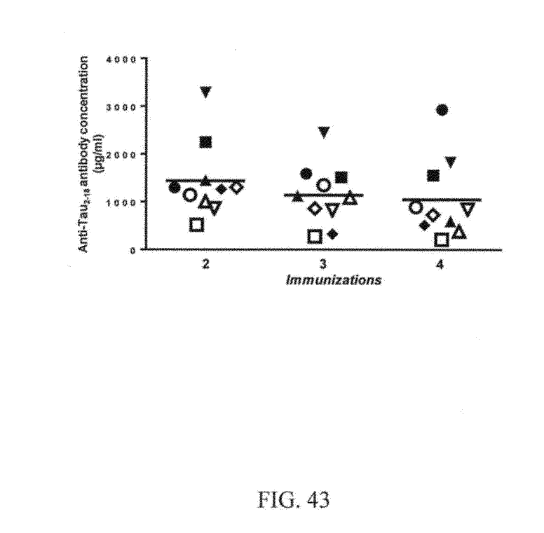

Blood was collected 14 days after each immunization and anti-tau antibody concentration was measured in sera by ELISA. AV-1980R/Advax^{CpG} induced very strong humoral responses in all vaccinated mice after second immunization reaching steady-state levels maintaining during the experiment (FIG. 43).

### Example 27

### Testing the Immunogenicity and Therapeutic Efficacy of Dual Vaccine Targeting Aβ and Tau in T5× Double Transgenic Mice

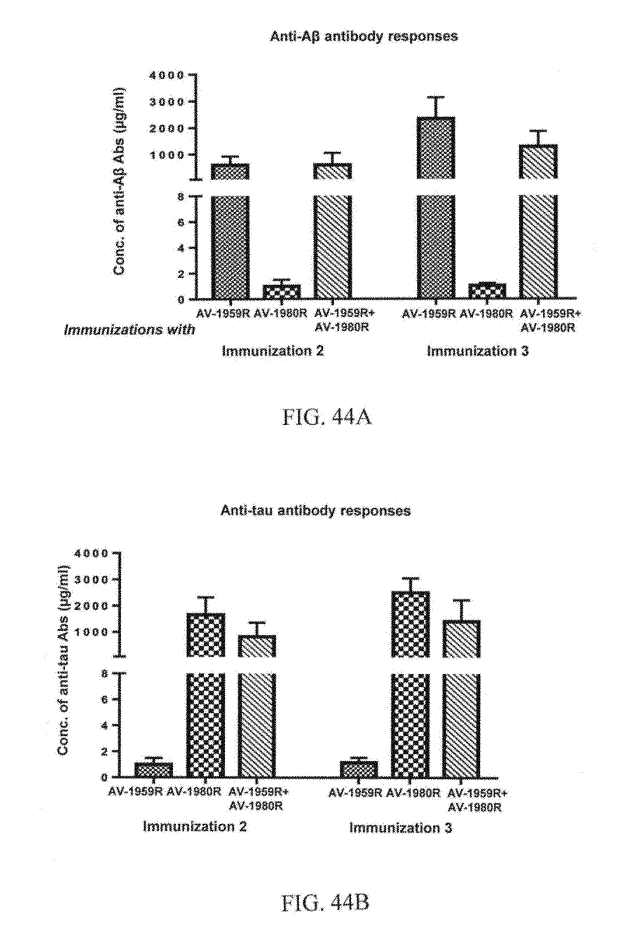

Three groups of 2.5-3 mo old male and female T5x APP/Tau double transgenic mice were immunized with AV-1959R, AV-1980R and combined AV-1959R+AV1980R vaccines, respectively. All vaccines have been formulated in Advax^{CpG} adjuvant. Mice from two control groups were injected with Advax^{CpG} only and PBS, respectively. Blood was collected 14 days after second immunization and anti-tau antibody concentration was measured in sera by ELISA. Both vaccines AV-1959R and AV-1980R formulated in Advax^{CpG} induced very strong anti-Aβ and anti-tau humoral immune responses, respectively, in all vaccinated mice after second immunization (FIGS. 44A-44B). Combined vaccine AV-1959R+AV1980R formulated in Advax^{CpG} induced production of anti-Aβ and anti-tau antibodies equal to concentrations in mice immunized with each vaccine separately.

### Example 28

### Testing the Immunogenicity AV-1980R Vaccine Targeting Tau Protein in RTG4510 Mouse Model of Tauopathy

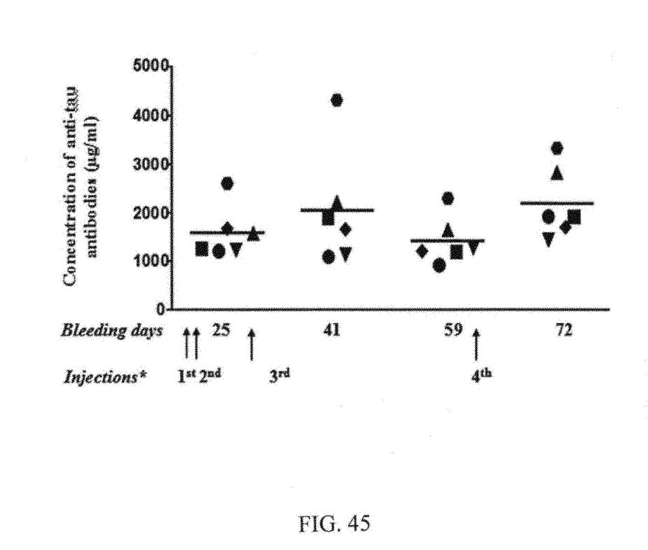

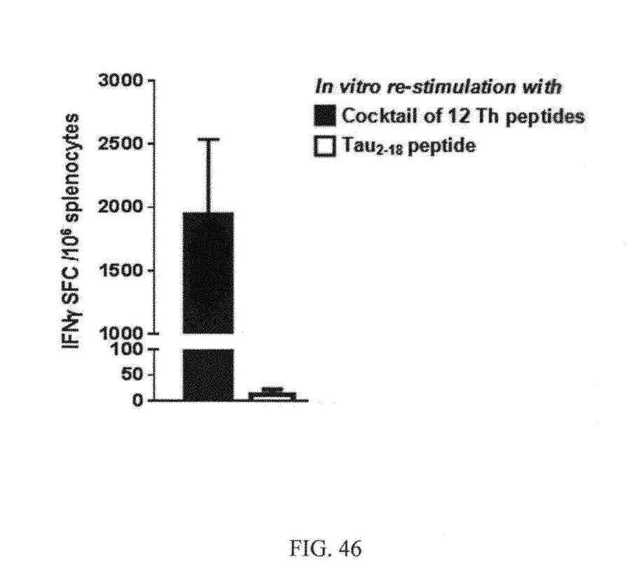

To determine the potency of Advax^{CpG} to enhance antibody responses in rTg4510 transgenic mice expressing mutant tau that could be suppressed with doxycycline, mice were immunized with AV-1980R formulated in Advax^{CpG} adjuvant and humoral and cellular responses were evaluated (FIG. 45 and FIG. 46).

Blood was collected 14 days after 2^{nd}, 3^{rd} and 4^{th} immunizations and anti-tau antibody concentration was measured in sera by ELISA. AV-1980R formulated in Advax^{CpG} induced very strong humoral responses in all vaccinated mice. Concentrations of anti-tau antibodies reached a peak after 2^{nd} immunization and persisted at the plateau to the end of the experiment (FIG. 45). At the end of the experiment, mice were sacrificed and cellular responses in the splenocytes of mice had been evaluated. In vitro re-stimulation of splenocytes with the cocktail of peptides incorporated into the MultiTEP platform induced the activation of high numbers of T cells measured by production of IFNγ by ELISPOT assay. Number of cells producing IFNγ was in background level in splenocytes re-stimulated with tau₂₋₁₈ peptide (FIG. 46).

### Example 29

### Testing the Immunogenicity AV-1950R Vaccine Targeting α-Syn Protein in Wild Type and Human α-Syn/Tg Mice

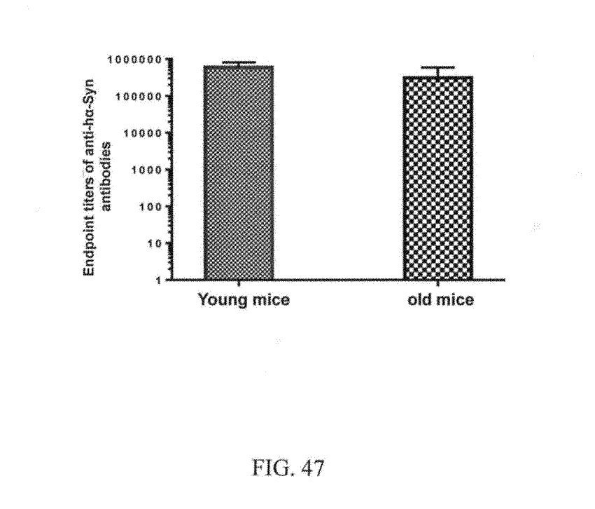

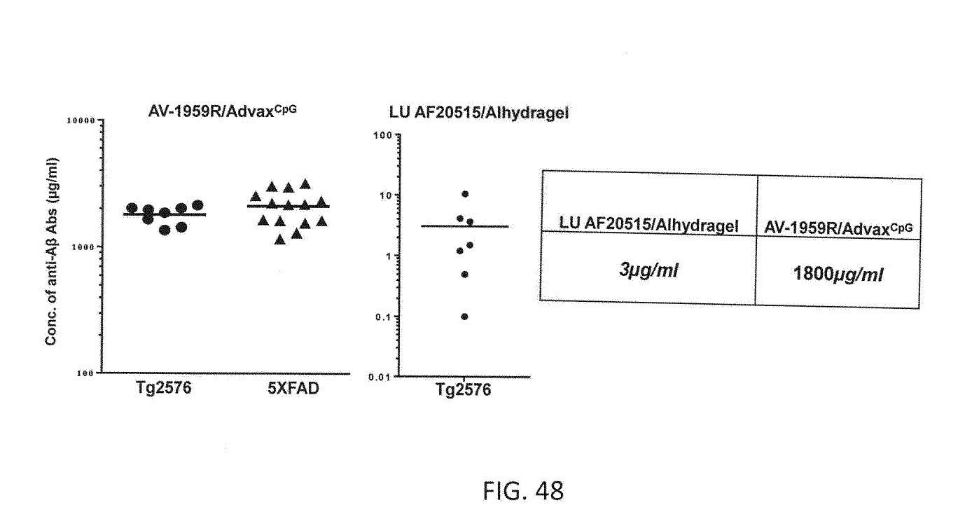

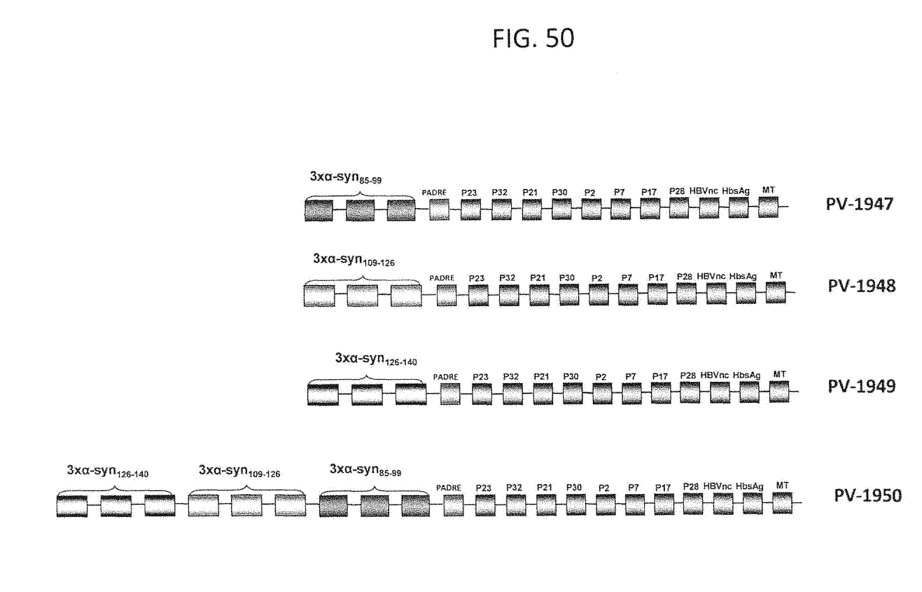

To determine the potency of Advax^{CpG} to enhance antibody responses to neuronal antigen human α-Syn (hα-Syn), the hallmark of LBD and PD, we prepared four MultiTEP-based vaccines targeting three B cell epitopes aa85-99 (PV-1947), aa109-126 (PV-1948), aa126-140 (PV-1949) separately or all of them together with reverse order (aa126-140+aa109-126+aa85-99; PV-1950) (FIG. 50). C57BL6 mice were immunized with each vaccine and titers of anti-ha-Syn antibodies were measured in sera collected after the 3^{rd} immunization. Although all vaccines induced high titers of antibodies, the strongest response was shown with vaccine that included three B cell epitopes together fused to MultiTEP. Endpoint titers of antibodies were 1:1.4×10⁶ for PV-1947R, 1:5×10⁵ for PV-1948R, 1:1.2×10⁶ for PV-1949R and 1:2.8×10⁶ for PV-1950R. Immunogenicity of PV-1950R targeting three epitopes of hα-Syn formulated in Advax^{CpG} were analyzed in hα-Syn/Tg mice (D line). Two cohorts of mice, young 3 month old and old 12-14 month old, have been immunized intramuscularly. Blood was collected 14 days after third immunization of young animals) and after second immunization of old mice and endpoint titers of anti-ha-Syn antibody were measured in sera by ELISA (FIG. 47). Mice in both cohorts generated high titers of antibodies specific to recombinant hα-Syn.

### Example 30

### Superiority of Aβ and Tau-Based Vaccines Formulated in Advax^{CPG} Adjuvant Vs Other Vaccines/Adjuvants Formulations in the Same Mouse Models

To test whether vaccine formulated in Advax^{CpG} adjuvant induced higher immune responses than vaccines formulated in other commonly used adjuvants, we compared the immunogenicity of our Aβ-based vaccine formulated in Advax^{CpG} adjuvant with Aβ-based vaccine LU AF20513 formulated in Alhydrogel in Tg2576 mice. The results were unexpectedly favorable for Advax^{CpG} adjuvant (FIG. 48). Surprisingly, Tg2576 mice that are known as immune compromised mice produced 600 time higher concentrations of anti-Aβ antibodies (1800 μg/mL) after immunization with vaccine d in Advax^{CpG} adjuvant compared with mice immunized with vaccine formulated in Alhydrogel (3 μg/mL). Vaccine formulated in Advax^{CpG} induce equally high titers of anti-Aβ antibodies in very aggressive 5×FAD Tg mice (FIG. 48).

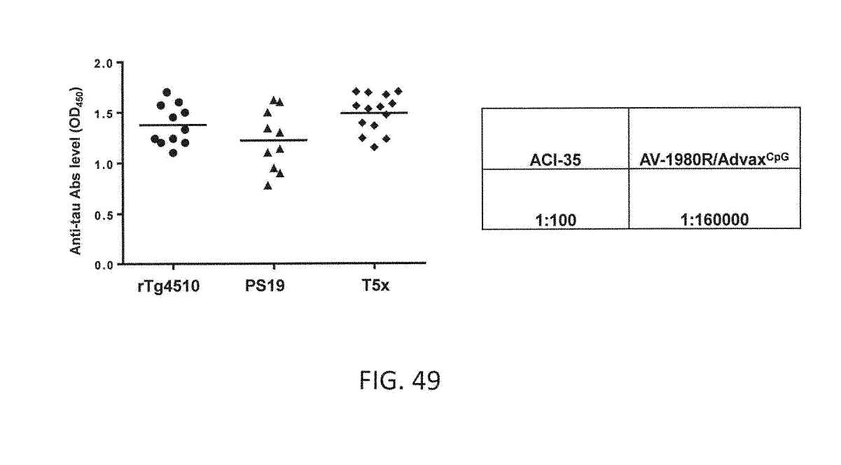

PS19 Tau/Tg mice were immunized with vaccine targeting tau protein (AV-1980R) formulated in Advax^{CpG} adjuvant and antibody titers have been compared with titers generated in the same strain of mice by liposome based ACI-35 Tau vaccine containing MPLA adjuvant presented in literature. The same OD detected in ELISA with ACI-35 anti-sera at dilution 1:100 was detected with anti-sera collected after immunization with AV-1980R/Advax^{CpG} at dilution 1:160000 (FIG. 49). In other words, the immune response against vaccine formulated in Advax^{CpG} adjuvant was 1600-fold higher compared with liposome based ACI-35 containing MPLA adjuvant. Such results were non-obvious and not expected.

It will be appreciated by persons skilled in the art that numerous variations and/or modifications may be made to the technology as shown in the specific embodiments without departing from the spirit or scope of the technology as broadly described. The present embodiments are, therefore, to be considered in all respects as illustrative and not restrictive.

What is claimed:

1. A vaccine composition comprising:
(a) inulin particles;
(b) a pathogen-associated molecular pattern (PAMP); and
(c) an antigen containing a protein or peptide derived from a neuronal self-antigen.

2. The vaccine composition of claim 1 wherein the inulin particles comprise delta inulin.

3. The vaccine composition of claim 1 wherein the inulin particles comprise a combination of delta inulin and aluminum phosphate or aluminum hydroxide.

4. The composition of claim 1 wherein the PAMP is a Toll-like receptor 9 ligand.

5. The composition of claim 1 wherein the protein or peptide derived from a neuronal self-antigen is Aβ, tau protein or α-synuclein or a peptide derived from Aβ, tau protein or α-synuclein.

6. The composition of claim 1 wherein the protein or peptide derived from a neuronal self-antigen is a sequence set forth in SEQ ID NO: 1 through SEQ ID NO: 45.

7. The composition of claim 1 wherein the antigen containing a protein or peptide derived from a neuronal self-antigen contains two or more repeated copies of the peptide derived from a neuronal self-antigen.

8. The composition of claim 1 wherein the antigen containing a protein or peptide derived from a neuronal self-antigen contains two or more repeated copies of two or more peptides derived from two or more neuronal self-antigens.

9. The composition of claim 1 wherein the protein or peptide derived from a neuronal self-antigen is expressed as a fusion protein with a synthetic protein sequence comprising one or more foreign epitopes for human CD4 T cells.

10. The composition of claim 9 wherein synthetic protein sequence comprising one or more foreign epitopes for human CD4 T cells is a sequence set forth in SEQ ID NO: 45.

11. The composition of claim 1 wherein the PAMP comprises an agonist recognized by one or more PRR (pattern recognition receptors).

12. The composition of claim 11 wherein the PRR is a Toll-like receptor (TLR), a RIG ligase, a NOD-like receptor, a C type Lectin or an RNA helices receptor.

13. The composition of claim 1 wherein the PAMP comprises RNA, DNA, an oligonucleotide or an unmethylated polynucleotide molecule.

14. The composition of claim 1, wherein the inulin particle comprises gamma inulin, delta inulin, epsilon inulin or omega inulin.

15. A method of preventing or treating a degenerative neurological disease in a subject, wherein said method comprises administering to the subject a therapeutically effective amount of the vaccine composition of claim 1.

16. The method of claim 15 where the degenerative neurological disease being prevented or treated is Alzheimer's disease or Parkinson's disease.

17. A method of manufacturing a vaccine, the method comprising the step of combining the components of claim 1 to produce a vaccine composition.


SEQUENCE LISTING (44 sequences)

SEQ ID NO: 1 - protein, 42 aa, Homo sapiens [sequence omitted]

SEQ ID NO: 2 - protein, 776 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSATKQVQRRPPPAGPRSERGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 4 - DNA, 126 bp, Homo sapiens
gatgcagaattccgacatgactcaggatatgaagttcatcatcaaaaattggtgttctttgcagaagatgtgggttcaaacaaaggtgcaatcattggactcatggtgggcggtgttgtcatagcg

SEQ ID NO: 5 - DNA, 2331 bp, Homo sapiens
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 6 - DNA, 423 bp, Homo sapiens
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 7 - protein, 6 aa, Homo sapiens
DAEFRH

SEQ ID NO: 8 - protein, 37 aa, Homo sapiens
AKAKTDHGAEIVYKSPVVSGDTSPRHLSNVSSTGSID

SEQ ID NO: 9 - protein, 18 aa, Homo sapiens
RSGYSSPGSPGTPGSRSR

SEQ ID NO: 10 - protein, 36 aa, Homo sapiens
NATRIPAKTPPAPKTPPSSGEPPKSGDRSGYSSPGS

SEQ ID NO: 11 - protein, 40 aa, Homo sapiens
GEPPKSGDRSGYSSPGSPGTPGSRSRTPSLPTPPTREPKK

SEQ ID NO: 12 - protein, 15 aa, Homo sapiens
KKVAVVRTPPKSPSS

SEQ ID NO: 13 - protein, 17 aa, Homo sapiens
AEPRQEFEVMEDHAGTY

SEQ ID NO: 14 - protein, 49 aa, Homo sapiens
KTKEGVLYVGSKTKEGVVHGVATVAEKTKEQVTNVGGAVVTGVTAVAQK

SEQ ID NO: 15 - protein, 15 aa, Homo sapiens
AGSIAAATGFVKKDQ

SEQ ID NO: 16 - protein, 18 aa, Homo sapiens
QEGILEDMPVDPDNEAYE

SEQ ID NO: 17 - protein, 15 aa, Homo sapiens
EMPSEEGYQDYEPEA

SEQ ID NO: 18 - protein, 5 aa, Homo sapiens
KAKEG

SEQ ID NO: 19 - protein, 15 aa, Homo sapiens
EMPSEEGYQDYEPEA

SEQ ID NO: 20 - protein, 15 aa, Homo sapiens
VFFAEDVGSNKGAII

SEQ ID NO: 21 - protein, 20 aa, Homo sapiens
QKLVFFAEDVGSNKGAIIGL

SEQ ID NO: 22 - protein, 14 aa, Homo sapiens
LVFFAEDVGSNKGA

SEQ ID NO: 23 - protein, 15 aa, Homo sapiens
QKLVFFAEDVGSNKG

SEQ ID NO: 24 - protein, 18 aa, Homo sapiens
GSNKGAIIGLMVGGVVIA

SEQ ID NO: 25 - protein, 15 aa, Clostridium tetani
VSIDKFRIFCKANPK

SEQ ID NO: 26 - protein, 16 aa, Clostridium tetani
LKFIIKRYTPNNEIDS

SEQ ID NO: 27 - protein, 15 aa, Clostridium tetani
IREDNNTLKLDRCNN

SEQ ID NO: 28 - protein, 21 aa, Clostridium tetani
FNNFTVSFWLRVPKVSASHLE

SEQ ID NO: 29 - protein, 14 aa, Clostridium tetani
QYIKANSKFIGITE

SEQ ID NO: 30 - protein, 20 aa, Clostridium tetani
LEYIPEITLPVIAALSIAES

SEQ ID NO: 31 - protein, 20 aa, Clostridium tetani
LINSTKIYSYFPSVISKVNQ

SEQ ID NO: 32 - protein, 20 aa, Clostridium tetani
NYSLDKIIVDYNLQSKITLP

SEQ ID NO: 33 - protein, 20 aa, Hepatitis B virus
PHHTALRQAILCWGELMTLA

SEQ ID NO: 34 - protein, 15 aa, Hepatitis B virus
FFLLTRILTIPQSLD

SEQ ID NO: 35 - protein, 16 aa, Influenza B virus
YSGPLKAEIAQRLEDV

SEQ ID NO: 36 - protein, 13 aa, Artificial Sequence
  synthesized fusion protein
AKFVAAWTLKAAA

SEQ ID NO: 37 - protein, 5 aa, Artificial Sequence
  synthesized fusion fragments
GSGSG

SEQ ID NO: 38 - protein, 4 aa, Artificial Sequence
  synthesized fusion fragments
YNGK

SEQ ID NO: 39 - protein, 7 aa, Artificial Sequence
  synthesized protein fragment with the sequence EAAAK repeated n times, where n=2,3,4 or 5
AEAAAKA

SEQ ID NO: 40 - protein, 13 aa, Artificial Sequence
  modified sequence
  MISC_FEATURE (1)..(1): D-alanine
  MISC_FEATURE (3)..(3): L-cyclohexylalanine
  MISC_FEATURE (3)..(3)
  MISC_FEATURE (13)..(13): D-alanine
XKXVAAWTLKAAX

SEQ ID NO: 41 - protein, 4 aa, Homo sapiens
EFRH

SEQ ID NO: 42 - protein, 20 aa, Homo sapiens
GKTKEGVLYVGSKTKEGVVH

SEQ ID NO: 43 - protein, 24 aa, Homo sapiens
EGVVHGVATVAEKTKEQVTNVGGA

SEQ ID NO: 44 - protein, 20 aa, Homo sapiens
EQVTNVGGAVVTGVTAVAQK